(19) United States
(12) Patent Application Publication
(10) Pub. No.: US 2025/0052742 A1
(43) Pub. Date: Feb. 13, 2025

(54) NOVEL FORMULATION FOR DRYING OF POLYMER DYE CONJUGATED ANTIBODIES

(71) Applicant: Beckman Coulter, Inc., Brea, CA (US)

(72) Inventors: Aditya JARARE, Brea, CA (US); Rajesh VENKATESH, Brea, CA (US); Sumeet CHAWLA, Brea, CA (US); Mehul JIVRAJANI, Brea, CA (US); Naina ARORA, Brea, CA (US); Gopinadh JAKKA, Brea, CA (US); Shiva Ranjini SRINIVASAN, Brea, CA (US)

(21) Appl. No.: 18/709,437

(22) PCT Filed: Nov. 12, 2021
(86) PCT No.: PCT/US2021/059251

§ 371 Date: May 10, 2024

(51) Int. Cl. G01N 33/53; C09B 67/20; C09B 69/10; C09K 11/02; C09K 11/06; G01N 15/10; G01N 15/14; G01N 33/533

(52) U.S. Cl. CPC G01N 33/5306; C09B 67/0066; C09B 69/10; C09K 11/025; C09K 11/06; G01N 15/14; G01N 33/533; C09K 2211/1416; G01N 2015/1006

(57) ABSTRACT

A novel dry down buffer is provided for use in drying a plurality of fluorescent dye conjugates on a substrate for use in flow cytometry. The aqueous buffer comprises a water-soluble monomer; a protein stabilizer; a carbohydrate stabilizer; and a zwitterionic surfactant. When mixed with a multi-color panel comprising fluorescent polymer dye conjugates, dried on a substrate, and reconstituted with a biological sample, the buffer provides decreased aggregation of fluorescent polymer dye conjugates, and decreased non-specific binding of monocytes and granulocytes, when compared to use of a buffer without the water-soluble monomer or zwitterionic surfactant.

This application is being filed on Nov. 12, 2021, as a PCT International Patent application.

### BACKGROUND

Multicolor flow cytometry is a rapidly evolving technology that uses multiple fluorescent markers to identify and characterize cellular subpopulations of interest, allowing rapid analysis on tens of thousands of cells per second. Flow cytometry uses antibody dye conjugates to stain different biological samples including whole blood samples, bone marrow, and other biological specimens. In a normal human whole blood sample, different types of cells express different markers, for example, CD4 in T-cells, and CD20 in B-cells. When mutually exclusive markers (markers expressed only in one particular cell type) are stained using their counter anti-marker antibody fluorescent dye conjugate, the signals (fluorescence) from each cell are captured and digitally converted in a flow cytometer for analysis.

Multicolor dry reagents are dried unitized cocktails comprising a multiplicity of different antibody dye conjugates useful in flow cytometry analysis of biological samples. Dry reagent technology is used to increase stability of biomolecules, allowing room temperature storage, simplify sample preparation, and minimize user errors.

Multicolor dry reagent cocktails may include several different antibodies conjugated to small molecule (e.g., FITC), tandem (e.g., PC5.5), or large molecule (e.g., APC) dye (e.g., CD4-FITC, CD8-PE, CD20-APC, PC5.5, etc.) and may be used to stain cells in various biological specimen and analyze it in a flow cytometer.

Compared to existing classical (monomer) dye (FITC, PC5.5, APC, etc.) conjugates, polymer dye conjugates are different in structure and complexity. Rigidity of polymer dyes structure helps to reduce rotational energy leading to brighter emission. Hence, polymer dye conjugates are particularly useful in identification and analysis of cells with scarcely expressed receptors. These bright polymer dyes can allow detection and resolution of dim population. However, polymer dyes such as violet polymer dyes, because of their inherent hydrophobic nature, tend to interact with each other and form aggregates.

Prior drying technology enables dry down of different classical dye conjugates in a single tube without altering functionality (affinity to antigens) or physical properties (such as brightness and spillover) with stability over a period of time. “Reagent Buffer” (RB) is a prior art formulation comprising sacrificial protein, a carbohydrate stabilizer, antimicrobial agent and buffering agent that was previously developed for drying different monomer dye conjugates in a single tube without altering functionality (affinity to antigens) or physical properties (such as brightness or fluorescence). “Physical properties” refers to brightness of the fluorescent dye conjugate and its spillover into other channels. For example, desirable flow cytometry results using CD4-PE dye conjugate are exhibited when dried using prior drying technology and used to stain biological specimen. Desirable functionality, physical properties, and resolution of CD4 PE+ monocytes and CD4 PE+ lymphocytes cell populations are exhibited, as shown in FIG. 1A. Similarly, desirable flow cytometry results are exhibited for CD20 APC dye conjugate when dried using prior technology, and reconstituted with a blood sample. Desirable functionality, physical properties, and flow cytometry resolution of CD20 APC+ cell population are exhibited, as shown in FIG. 1B.

When prior drying technology using Reagent Buffer was used to dry down two polymer dye antibody conjugates in a tube, it could not prevent the non-specific interaction between the polymer dye conjugates, resulting in aggregation. In general, when two or more polymer dye antibody conjugates are dried using the prior drying technology, they tend to interact with each other leading to undesired results. Aggregation results in false positive population in other channels that cannot be corrected. This problem is illustrated in FIG. 2A and FIG. 2B.

FIG. 2A shows undesirable flow cytometry results of CD20-605 and CD4-786 polymer dye antibody conjugates when mixed and dried using prior drying technology. Inability to resolve populations on x and y axis was assumed to be a spillover problem. However, even after correction of spillover, the populations were not resolved (FIG. 2B).

FIG. 2B shows undesirable flow cytometry results of CD20-605 and CD4-786 polymer dye antibody conjugates when mixed and dried using prior drying technology with spillover correction. The inability to correct the populations to their respective fluorescence channel was deemed to be due to aggregation of polymer dye antibody conjugates when two polymer dye antibody conjugates were dried using prior drying technology.

A need exists for a novel buffer formulation which can keep polymer dye antibody conjugate stable and avoid aggregation while drying and during reconstitution, allow for improved functionality, physical properties, and flow cytometry resolution.

### SUMMARY OF THE DISCLOSURE

A novel buffer composition is provided for use in drying a plurality of dye conjugates on a substrate. The dye conjugates may comprise a fluorescent dye conjugate. The fluorescent dye conjugate may be the conjugate of a fluorescent dye and a binding partner, such as an antibody. The fluorescent dye conjugate may be a fluorescent polymer dye conjugated to a binding partner, such as an antibody. The fluorescent dye conjugates may be used in flow cytometry. The buffer composition comprises a water-soluble monomer; a protein stabilizer; a carbohydrate stabilizer; and a zwitterionic surfactant. When the buffer composition is mixed with a multi-color fluorescent dye conjugate panel comprising two or more fluorescent dye conjugates, dried on a substrate, and reconstituted with a biological specimen, the buffer provides decreased aggregation of fluorescent dye conjugates, when compared to use of a buffer without the protein stabilizer, water-soluble monomer or zwitterionic surfactant. In some instances, the buffer composition also provides decreased non-specific binding of the dye to monocytes and/or decreased non-specific binding of the dye to granulocytes, when compared to use of a buffer without the protein stabilizer, monomer or zwitterionic surfactant.

In some embodiments, the disclosure provides a composition comprising multiple fluorescent dye conjugates in a panel. In presence of an appropriate buffer, multiple fluorescent dye conjugates can be used in a panel to identify cell subpopulations. Without appropriate buffer, fluorescent dye conjugates might interact with each other and cause staining artifacts which may affect data interpretation.

A buffer composition is provided for use in drying a plurality of dye conjugates on a substrate, comprising a water-soluble monomer; a protein stabilizer; a carbohydrate stabilizer; and a zwitterionic surfactant. At least one of, at least two of, or at least three of the plurality of dye binding partner conjugates may comprise a fluorescent polymer dye moiety.

The water-soluble monomer may be a monomeric unit comprising an aryl moiety or heteroaryl moiety, each optionally having a water-soluble moiety attached thereto. The water-soluble moiety may be one or more poly(ethylene glycol) moieties. The water-soluble monomer may be appropriate for use in preparation of at least one of the plurality of the fluorescent polymer dyes having a monomer A subunit, a monomer B subunit, or a combination of monomer A and monomer B subunits. The water-soluble monomer may be a dihydrophenanthrene (DHP)-based water-soluble monomer. The water-soluble monomer may be a fluorene-based water-soluble monomer.

The water-soluble monomer may be a dihydrophenanthrene (DHP)-based monomer having a chemical structure according to Formula (I):

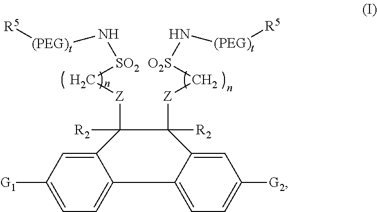

wherein
each G₁, G₂ is independently selected from the group consisting of halogen, alkyl, PEG, hydrogen, alkyne, optionally substituted aryl, optionally substituted heteroaryl, halogen substituted aryl, silyl, diazonium salt, triflate, acetyloxy, azide, sulfonate, phosphate, optionally substituted tetrahydropyrene (THP), optionally substituted fluorene, optionally substituted dihydrophenanthrene (DHP), aryl or heteroaryl substituted with one or more pendant chains terminated with a functional group selected from amine, carbamate, carboxylic acid, carboxylate, maleimide, activated ester, N-hydroxysuccinimidyl, hydrazine, hydrazide, hydrazone, azide, alkyne, aldehyde, and thiol;
each R² is independently selected from the group consisting of a water-solubilizing moiety, alkene, alkyne, cycloalkyl, haloalkyl, (hetero)aryloxy, (hetero)arylamino, sulfonamide-PEG, phosphoramide-PEG, ammonium alkyl salt, ammonium alkyloxy salt, ammonium oligoether salt, sulfonate alkyl salt, sulfonate alkoxy salt, sulfonate oligoether salt, sulfonamido oligoether, sulfonamide, sulfinamide, phosphonamidate, phosphinamide,

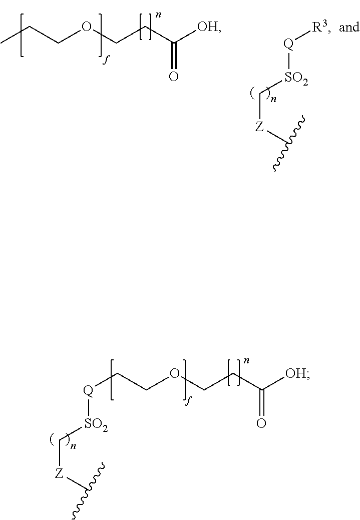

each R³ is a water-solubilizing moiety;
each R⁴ is independently selected from the group consisting of H, alkyl, PEG, a water-solubilizing moiety, a linker moiety, a chromophore, carboxylic amine, amine, carbamate, carboxylic acid, carboxylate ester, maleimide, activated ester, N-hydroxysuccinimidyl, hydrazine, hydrazide, hydrazone, azide, alkyne, aldehyde, or thiol, or protected groups thereof;
each R⁵ is independently selected from the group consisting of H, hydroxyl, C₁-C₁₂ alkyl, C₂-C₁₂ alkene, C₂-C₁₂ alkyne, C₃-C₁₂ cycloalkyl, C₁-C₁₂ haloalkyl, C₁-C₁₂ alkoxy, C₂-C₁₈ (hetero)aryloxy, C₂-C₁₈ (hetero)arylamino, C₂-C₁₂ carboxylic acid, C₂-C₁₂ carboxylate ester, and C₁-C₁₂ alkoxy;
each Q is independently a bond, NR⁴, or —CH₂;
each Z is independently CH₂, O, or NR⁴;
each f is independently an integer from 0 to 50; and
each n is independently an integer from 1 to 20.

In some embodiments, each G₁, G₂ is independently selected from the group consisting of halo (F, Cl, Br, I), C₁-C₆ alkyl, and PEG.

The water-soluble monomer may be a fluorene-based monomer having a chemical structure according to Formula (II):

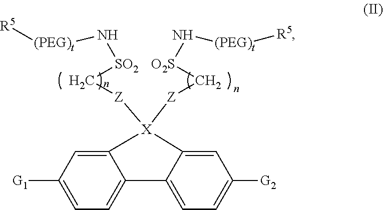

wherein
each G₁, G₂ is independently selected from the group consisting of halogen, alkyl, PEG, alkyne, hydrogen, optionally substituted aryl, optionally substituted heteroaryl, halogen substituted aryl, silyl, diazonium salt, triflate, acetyloxy, azide, sulfonate, phosphate, optionally substituted tetrahydropyrene (THP), optionally substituted fluorene, optionally substituted dihydrophenanthrene (DHP), aryl or heteroaryl substituted with one or more pendant chains terminated with a functional group selected from amine, carbamate, carboxylic acid, carboxylate, maleimide, activated ester, N-hydroxysuccinimidyl, hydrazine, hydrazide, hydrazone, azide, alkyne, aldehyde, and thiol;
each X is C or Si;
each R⁴ is independently selected from the group consisting of H, alkyl, PEG, a water-solubilizing moiety, a linker moiety, a chromophore, carboxylic amine, amine, carbamate, carboxylic acid, carboxylate ester, maleimide, activated ester, N-hydroxysuccinimidyl, hydrazine, hydrazide, hydrazone, azide, alkyne, aldehyde, or thiol, or protected groups thereof;
each R⁵ is independently selected from the group consisting of H, hydroxyl, C₁-C₁₂ alkyl, C₂-C₁₂ alkene, C₂-C₁₂ alkyne, C₃-C₁₂ cycloalkyl, C₁-C₁₂ haloalkyl, C₁-C₁₂ alkoxy, C₂-C₁₈ (hetero)aryloxy, C₂-C₁₈ (hetero)arylamino, C₂-C₁₂ carboxylic acid, C₂-C₁₂ carboxylate ester, and C₁-C₁₂ alkoxy;
each Z is independently CH₂, O, or NR⁴;
each f is independently an integer from 0 to 50; and
each n is independently an integer from 1 to 20.

In some embodiments, each G₁, G₂ is independently selected from the group consisting of halo (F, Cl, Br, I), C₁-C₆ alkyl, and PEG.

The water-soluble monomer may be a dihydrophenanthrene (DHP)-based monomer having a chemical structure according to Formula (III):

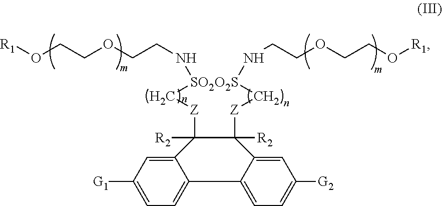

wherein each G₁, G₂ is independently halo (F, Cl, Br, I); each Z is independently selected from the group consisting of O, CH₂, and NH; each R₁ is independently alkyl (C₁-C₃); each R₂ is independently H, alkyl(C₁-C₆); each n is independently 1-6; and each m is independently 5-50. In some embodiments, G₁, G₂ are each Br; each Z is O;
each R₁ is CH₃; each R² is H; each n is independently 2-4; and each m is independently 5-20. In some embodiments, each n is 3; and each m is 11 or 12.

The protein stabilizer may be selected from one or more of the group consisting of a casein, a bovine serum albumin (BSA), and a gelatin.

The carbohydrate stabilizer may be a disaccharide carbohydrate stabilizer. The disaccharide carbohydrate stabilizer may be a trehalose, sucrose, maltose, cellobiose, or melibiose, or a hydrate thereof. In specific embodiments, the disaccharide carbohydrate stabilizer may be a trehalose or a hydrate thereof. The carbohydrate stabilizer may be trehalose dihydrate.

The zwitterionic surfactant may comprise a structure according to Formula (XV):

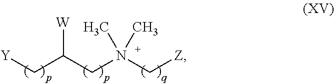

wherein Y═CO₂—, or SO₃—, W═H or OH, Z═CH₃ or NHC(O)R, wherein R═C₁₋₁₅ alkyl; independently each p=0 or 1; and q=0-21; optionally wherein W═H, Z═CH₃, and q=11-15.

The zwitterionic surfactant may be selected from the group consisting of 3-(N,N-dimethylmyristylammonio propane sulfonate (DMMA); 3-[N,N-dimethyl (3-palmitoylaminopropyl) ammonio]-propane sulfonate (DMPA); N-(alkyl C₁₀-C₁₆)—N,N-dimethylglycine betaine; and N,N-dimethyl-N-dodecylglycine betaine.

Optionally the buffer composition may include one or more, two or more, or three or more additional additives selected from the group consisting of a preservative, antioxidant, anionic surfactant, nonionic surfactant, and a colorant.

The buffer composition may have a pH with a range of pH 6.5-7.5. In some instances, the aqueous buffer composition may have a pH within a range of pH 7.0-7.4.

The buffer composition may comprise per test: 200 to 800 μg of the monomer; 2000 to 3000 μg of the carbohydrate stabilizer; 8.4 to 72 μg of the protein stabilizer; and 2 to 15 μg of the zwitterionic surfactant.

The buffer composition may comprise per test: 300 to 600 μg of the monomer; 2200 to 2800 μg of the carbohydrate stabilizer; 15 to 20 μg of the protein stabilizer; and 8 to 12 μg of the zwitterionic surfactant.

In some embodiments, the buffer composition may include a plurality of fluorescent dye conjugates. In some embodiments, the buffer composition may include a plurality of fluorescent polymer dye conjugates. The fluorescent polymer dye conjugates may comprise a structure according to Formula (V), Formula (VI), Formula (VII), Formula (VIII), Formula (IX), Formula (X), Formula (XI), Formula (XII), Formula (XIII), and/or Formula (XIV), each according to the disclosure.

In other embodiments, the aqueous buffer composition does not include a plurality of fluorescent dye conjugates. In further embodiments, the aqueous buffer composition does not include any fluorescent dye conjugates.

A novel method of preparing a single reactant film is provided, the method comprising dispensing onto a substrate a plurality of reactants together in a liquid phase comprising the buffer composition according to the disclosure, the plurality of reactants comprising a first reactant and a second reactant, the first reactant including a first binding partner conjugated to a first dye, wherein the first dye comprises a fluorescent polymer dye; the second reactant including a second binding partner conjugated to a second dye; and drying the first reactant and the second reactant together in the liquid phase aqueous buffer to form a first single reactant film on the substrate. The second dye may be a fluorescent polymer dye. The single reactant film may be a uniform film that includes a plurality of reactants. The plurality of reactants may include two or more, three or more, four or more, five or more, six or more, seven or more, eight or more, nine or more, 10 or more, 11 or more, or 12 or more reactants, or 2 to 20, 3 to 18, or 4 to 12 different reactants each comprising a different binding partner dye conjugate. The dye may be a fluorescent dye. The fluorescent dye may be a fluorescent polymer dye. For example, each reactant may be a unique fluorescent dye conjugate. The plurality of reactants may include two or more unique fluorescent polymer dye conjugates. The substrate may be a tube, well, membrane, or bead. The substrate may comprise an inside surface of a reaction vessel.

The disclosure provides a single reactant film that is a uniform film comprising a plurality of reactants prepared by the novel method of the disclosure. The single reactant film will, after exposing to a first aliquot of a liquid biological sample, processing, and analyzing by flow cytometry, provide a first flow cytometry plot exhibiting one or more of decreased non-specific binding of monocytes; decreased non-specific binding of granulocytes; decreased non-specific interaction of fluorescent dye conjugates; decreased aggregation of fluorescent dye conjugates, when compared to a second flow cytometry plot obtained by exposing a second single reactant film comprising the first reactant and the second reactant, to a second aliquot of the liquid biological sample, processing, and analyzing by flow cytometry, wherein the second single reactant film is prepared with a liquid phase of prior technology without the water-soluble monomer, and without the zwitterionic surfactant. The fluorescent dye conjugate may be a fluorescent polymer dye conjugate.

The buffer composition according to the disclosure enables drying different fluorescent dye conjugates in a single tube to provide a uniform film without altering functionality (i.e., affinity to an analyte) and physical properties (such as brightness or fluorescence), while avoiding aggregation and consequent false positive population into other channels.

### BRIEF DESCRIPTION OF THE DRAWINGS

FIG. 1A shows flow cytometry density plot of CD4-PE conjugate when dried using prior drying technology Reagent Buffer. Desirable functionality (affinity to antigens), physical properties (such as brightness or fluorescence), and resolution of cell populations are exhibited.

FIG. 1B shows flow cytometry density plot of CD20-APC conjugate when dried using prior drying technology Reagent Buffer. Desirable functionality (affinity to antigens) and physical properties (such as brightness or fluorescence) and resolution of cell populations are exhibited.

FIG. 2A shows flow cytometry two-dimensional dot plot of CD20-SNv605 and CD4-SNv786 polymer dye conjugates when mixed and dried using prior drying technology Reagent Buffer without compensation. Undesirable results and an inability to resolve populations on x and y axis was exhibited.

FIG. 2B shows flow cytometry results of CD20-SNv605 and CD4-SNv786 polymer dye conjugates when mixed and dried using prior drying technology Reagent Buffer with compensation. The inability to compensate population was deemed to be due to aggregation of polymer dye antibody conjugates when the two polymer dyes were dried using prior drying technology.

FIG. 3A shows flow cytometry two-dimensional dot plot of CD20-SNv605 and CD4-SNv786 polymer dye conjugate dry down in DM 2 with stabilizer (DM2+S) DM buffer without compensation. Spillover of 610 fluorescent channel is observed in 780 fluorescent channel.

FIG. 3B shows flow cytometry two-dimensional dot plot of CD20-SNv605 and CD4-SN v 786 polymer dye conjugate dry down in inventive DM buffer DM2+S with compensation. The DM2+S DM buffer can resolve cell population without any non-specific interaction.

FIG. 4 shows chemical structures of representative Monomer A and Monomer B.

FIG. 5 shows flow cytometry plots for dose titration of Casein in DM using CD20-SNv605 to measure percentage recruitment and demonstrate the reduction in non-specific events.

FIG. 6 shows two-dimensional fluorescence dot plots of dry formulation for 3 color testing at 200 μg (top panels) and 400 μg per test (bottom panels) of monomer A concentration. Reduced spread of populations was observed at 400 μg per test of monomer A.

FIG. 7A shows flow plots of scatter properties in dose optimization of DMMA. FIG. 7A shows unstained (upper left), dry DM alone (second from left, upper panel), dry DMMA 0.004% in DM (third from left, upper panel), dry DMMA 0.008% in DM (upper right panel), dry DMMA 0.018% in DM (lower left panel), dry DMMA 0.021% in DM (lower middle panel), and dry DMMA 0.03% in DM (lower right panel). DMMA at 0.004% and 0.008% shows less non-specific neutrophil pullout (indicated by arrows) as compared to other DMMA concentrations.

FIG. 7B shows CD20-SNv605 dot plots for all the formulations for dose optimization of DMMA in DM including unstained (upper left), dry DM alone (second from left, upper panel), dry DMMA 0.004% in DM (third from left, upper panel), dry DMMA 0.008% in DM (upper right panel), dry DMMA 0.018% in DM (lower left panel), dry DMMA 0.021% in DM (lower middle panel), and dry DMMA 0.03% in DM (lower right panel). DMMA at 0.004%, 0.008% and 0.018% shows less non-specific monocyte pullout in 610 channel (indicated by arrows) as compared to DM and other DMMA concentrations.

FIG. 7C shows HLADR-786 dot plots for all the formulations for dose optimization of DMMA in DM including unstained (upper left), dry DM alone (second from left, upper panel), dry DMMA 0.004% in DM (third from left, upper panel), dry DMMA 0.008% in DM (upper right panel), dry DMMA 0.018% in DM (lower left panel), dry DMMA 0.021% in DM (lower middle panel), and dry DMMA 0.03% in DM (lower right panel).

FIG. 7D show CD20-605 and HLADR-786 two-dimensional dot plots for dry DM alone (upper left panel), dry DMMA 0.004% in DM (upper middle panel), dry DMMA 0.008% in DM (upper right panel), dry DMMA 0.018% in DM (lower left panel), dry DMMA 0.021% in DM (lower middle panel), and dry DMMA 0.03% in DM (lower right panel).

FIG. 8A shows flow plots of scatter properties for a combination of additives with 0.008% DMMA, including unstained (upper left), dry DM (second from left, upper panel), DMMA 0.008% (upper right), DMMA 0.008%+Casein 2.5× (lower left), DMMA 0.008%+Prionex 2 dil (lower middle panel), and DMMA 0.008%+Prionex 3 dil (lower right). Scatter properties look similar for each of the combinations, except DM which shows non-specific granulocyte and monocyte pullout as indicated with arrows.

FIG. 8B shows CD20-SNv605 dot plots for a combination of additives with 0.008% DMMA, including single CD20 (upper left), dry DM (second from left, upper panel), DMMA 0.008% (upper right), DMMA 0.008%+Casein 2.5× (lower left), DMMA 0.002%+Prionex 2 dil (lower middle panel), and DMMA 0.008%+Prionex 3 dil (lower right).

FIG. 8C shows HLADR-786 dot plots for a combination of additives with 0.008% DMMA, including single HLADR (upper left), dry DM (second from left, upper panel), DMMA 0.008% (upper right), DMMA 0.008%+Casein 2.5× (lower left), DMMA 0.008%+Prionex 2 dil (lower middle panel), and DMMA 0.008%+Prionex 3 dil (lower right). % recruitment of HLADR+ was found to be similar in all the combination with 0.008% DMMA compared to respective liquid singles.

FIG. 8D shows CD20-SNv 605 and HLADR-786 two-dimensional dot plots for combinations of additives with 0.008% DMMA, including dry DM (upper left panel), DMMA 0.008% (upper right), DMMA 0.002%+Casein 2.5× (lower left), DMMA 0.008%+Prionex 2 dil (lower middle panel), and DMMA 0.008%+Prionex 3 dil (lower right). % recruitment of dual positive population in all the combination with 0.008% DMMA was found to be similar as compared to DM.

FIG. 9 shows physical appearance and properties of dry tubes in each of the tested groups DM2+S, Trehalose+Monomer, Trehalose+Casein, Trehalose+DMMA (left to right, upper panel), DM2+S, DM2+S without Monomer, DM2+S without DMMA, DM2+S without casein (left to right, lower panel). Here, DM2+S serves as control group. Physical observation indicates that without monomer, there is change in color of dry film (typically red film becomes light orange to brown). Shrinkage of film was observed in groups without casein and DMMA. No change in appearance of dry film was observed in tubes without DMMA compared to DM2+S. However, tubes without casein shows marginal shrinkage of dry film.

FIG. 10A shows side scatter SSC vs FL plots for CD56-SNv428 in tested groups DM2+S, DM2+S without DMMA, DM2+S without casein, DM2+S without Monomer (left to right, top panel), Trehalose+DMMA, Trehalose+Casein, Trehalose+Monomer (left to right, bottom panel). When casein and DMMA are not present, there is non-specific monocyte pullout (indicated with arrows). Absence of monomer causes spread of negative population in lymphocyte (indicated with arrows). Spread of negative population is mainly attributed to non-specific interaction among SN dyes in absence of monomer and casein.

FIG. 10B shows side scatter SSC vs FL plots for CD20-SNv605 in tested groups DM2+S, DM2+S without DMMA, DM2+S without casein, DM2+S without Monomer (left to right, top panel), Trehalose+DMMA, Trehalose+Casein, Trehalose+Monomer (left to right, bottom panel). When casein is not present, there is non-specific monocyte pullout (indicated with arrows). Absence of monomer causes spread of negative population in lymphocyte (indicated with arrows). Spread of negative population is mainly attributed to non-specific interaction among SN dyes in absence of monomer and casein.

FIG. 10C shows side scatter SSC vs FL plots for CD4-SNv786 in tested groups DM2+S, DM2+S without DMMA, DM2+S without casein, DM2+S without Monomer (left to right, top panel), Trehalose+DMMA, Trehalose+Casein, Trehalose+Monomer (left to right, bottom panel). Absence of monomer causes spread of negative population in lymphocyte (indicated with arrows).

FIG. 10D shows two-dimensional flow plots for CD56-SNv428 vs CD20-SNv605 in tested groups DM2+S, DM2+S without DMMA, DM2+S without casein, DM2+S without Monomer (left to right, top panel), Trehalose+DMMA, Trehalose+Casein, Trehalose+Monomer (left to right, bottom panel). Absence of monomer and casein cause non-specific interaction/spread of population (indicated with arrows) among the SN population. This plot shows that both monomer and casein are important to prevent non-specific interaction.

FIG. 10E shows two-dimensional flow plots for CD4-SNv786 vs CD20-SNv605 in tested groups DM2+S, DM2+S without DMMA, DM2+S without casein, DM2+S without Monomer (left to right, top panel), Trehalose+DMMA, Trehalose+Casein, Trehalose+Monomer (left to right, bottom panel). Absence of monomer and casein cause non-specific interaction/spread of population (indicated with arrows) among the SN population. This plot illustrates that both monomer and casein are important to prevent non-specific interaction.

FIG. 10F shows two-dimensional flow plots for CD4-SNv786 vs CD56-SNv428 in tested groups DM2+S, DM2+S without DMMA, DM2+S without casein, DM2+S without Monomer (left to right, top panel), Trehalose+DMMA, Trehalose+Casein, Trehalose+Monomer (left to right, bottom panel). Absence of monomer and casein cause non-specific interaction/spread of population (indicated with arrows) among the SN population. Hence both monomer and casein are important to prevent non-specific interaction.

FIG. 11A shows representative SSC vs FL overlay flow plots for three polymer dye conjugates CD56-SNv428, CD20-SNv605, and CD4-SNv786 including gating CD45-APC-A750, dried in a cocktail with inventive DM2+S dry down buffer and reconstituted with blood sample. In a second group, liquid cocktail of same antibodies was prepared using commercially available BD Horizon™ Brilliant Stain buffer. BD Horizon™ Brilliant stain buffer caused non-specific granulocyte and monocyte pullout (indicated with arrow) as compared to DM2+S dry tubes.

FIG. 11B shows representative two-dimensional overlay flow plots for three polymer dye antibody conjugates CD56-SNv428, CD20-SNv605, and CD4-SNv786 dried with inventive DM2+S dry down buffer and reconstituted with blood sample. In a second group, liquid cocktail of same antibodies was prepared using commercially available BD Horizon™ Brilliant Stain buffer. The BD Horizon™ Brilliant stain buffer caused non-specific lymphocyte pull-out in all the combination of SN conjugates (for, e.g., non-specific lymphocyte pull-out in CD56 and CD20), when compared to DM2+S.

FIG. 12 shows photograph of DM2+S buffer dry tubes at 6-months old, open pouch.

FIG. 13A shows fresh, 3-month old, and 6-month old DM2+S dry tubes 6-months stability. Representative two-dimensional overlay flow plots for different combination of SN dyes in each of the three lots is shown. The dry tubes contain 12 different binding partner dye conjugates; i.e., a 12 color (12C) panel (9C conventional conjugates+3C SN conjugates, CD56-SNv428, CD20-SNv605, CD4-SNv786). These lots were tested on 4 donors with single replicate on one flow cytometer instrument. Stain-lyse-wash protocol mentioned in the protocols and methods was used for the study. These overlays shows that population in 3 month and 6 month aged lots completely overlay with fresh lot. In addition, no non-specific interaction or spread of population was observed in 6-month aged lot in all the tested donors.

FIG. 13B shows DM2+S dry tubes 6-months stability compared to 3-months, and fresh lots: Representative two-dimensional overlay flow plots for SN vs Classical combination in all the three lots. Dry tubes containing a 12 color (12C) panel (9C conventional conjugates+3C SN conjugates, CD56-SNv428, CD20-SNv605, CD4-SNv786) were tested on 4 donors with single replicate on one flow cytometry instrument. Overlays flow plots for 6-months and 3-months aged lot with fresh lots for CD3 ECD-A vs CD20Violet610-A (left panel), CD8 KO525-A vs CD4Violet780-A (middle panel), and CD45 APC-A750-A vs CD56PB450-A (right panel) shows that population in 3-month and 6-month aged lots completely overlay with fresh lot. In addition, no non-specific interaction or spread of population was observed in 6-month old lot in all the tested donors.

FIG. 13C shows DM2+S dry tubes 6-months stability compared to 3-months and fresh lots: Representative two-dimensional overlay flow plots for Classical vs Classical combination in all the three lots. Dry tubes containing a 12 color (12C) panel (9C conventional conjugates+3C SN conjugates, CD56-SNv428, CD20-SNv605, CD4-SNv786) were tested on 4 donors with single replicate on one flow cytometry instrument. Overlays for 6-months and 3-months aged lots with fresh lots for CD16 FITC-A vs CD25PE-A (left panel), CD8 KO525-A vs CD45APC-A750-A (middle panel), and CD3 ECD-A vs CD10APC-A (right panel) shows that population in 3-month and 6-month aged lots completely overlay with fresh lot. In addition, no non-specific interaction or spread of population was observed in 6-month old lot in all the tested donors.

### DETAILED DESCRIPTION OF THE DISCLOSURE

The present disclosure provides compositions and methods to minimize aggregation of two or more dye conjugates when dried down together. A dry down buffer composition is provided that helps maintain integrity of fluorescent dye structure in a mixture of fluorescent dye conjugates, decreases aggregation, and decreases non-specific interaction and staining artifacts. For example, the dry down buffer composition may be used to decrease or prevent aggregation of two or more fluorescent polymer dye conjugates when dried down together or during reconstitution.

Aggregation among fluorescent dye conjugates may occur in the liquid cocktail or while drying a cocktail of fluorescent dye conjugates or during reconstitution. The present invention solves the problem by providing a dry down buffer composition to decrease or avoid aggregation, for example, so that fluorescent polymer dye conjugates do not interact during drying. Upon reconstitution, the fluorescent dye conjugates can independently bind to target analytes in the liquid sample to be analyzed and erroneous results arising from aggregation or cross-linking are substantially reduced or eliminated.

A novel dry down buffer has been developed which can decrease or eliminate non-specific interaction of fluorescent dyes and also does not hamper natural binding capacity of fluorescent dye conjugates to the antigen of interest.

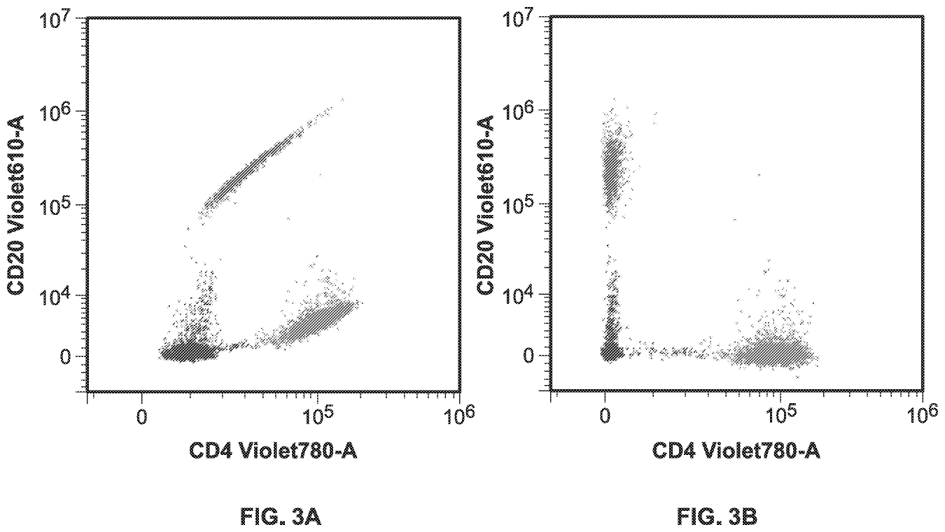

FIG. 2B and FIG. 3B shows comparative flow cytometry dot plots of CD20-605 and CD4-786 polymer dye conjugate dried down using (FIG. 2B) prior dry down technology, and (FIG. 3B) inventive dry down (“DM”) buffer DM2+S tubes and processed according to Example 1. FIG. 2B shows that polymer dye conjugate dried down using prior dry down technology fails to resolve the cell population, whereas FIG. 3B shows that inventive DM buffer DM2+S can resolve cell population without any non-specific interaction.

### Definitions

As used herein, the following terms and variations thereof have the meanings given below, unless a different meaning is clearly intended by the context in which such term is used.

The singular forms “a”, “an” and “the” are intended to include the plural forms as well, unless the context clearly indicates otherwise.

The term “and/or” refers to and encompasses any and all possible combinations of one or more of the associated listed items.

The term “about,” when referring to a measurable value such as an amount of a compound, dose, time, temperature, and the like, is meant to encompass variations of 10%, 5%, 1%, 0.5%, or even 0.1% of the specified amount, and at least industry-standard variation in the test method for measuring the value.

The terms, “patient”, “subject” or “subjects” include but are not limited to humans, the term may also encompass other mammals, or domestic or exotic animals, for example, dogs, cats, ferrets, rabbits, pigs, horses, cattle, birds, or reptiles.

Unless otherwise specified, the term phrase “room temperature” refers to 18 to 27° C.

Unless otherwise specified, the term “percent”, or “%” refers to weight percent.

The term “activated ester” or “active esters” by itself or as part of another substituent refers to carboxyl-activating groups employed in peptide chemistry to promote facile condensation of a carboxyl group with a free amino group of an amino acid derivative. Descriptions of these carboxyl-activating groups are found in general textbooks of peptide chemistry, for example K. D. Kopple, “Peptides and Amino Acids”, W. A. Benjamin, Inc., New York, 1966, pp. 50-51 and E. Schroder and K. Lubke, “The Peptides”; Vol. 1, Academic Press, New York, 1965, pp. 77-128.

The term “acyl” as used herein refers to a group containing a carbonyl moiety wherein the group is bonded via the carbonyl carbon atom. The carbonyl carbon atom is bonded to a hydrogen forming a “formyl” group or is bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. An acyl group can include 0 to about 12, 0 to about 20, or 0 to about 40 additional carbon atoms bonded to the carbonyl group. An acyl group can include double or triple bonds within the meaning herein. An acyl group can optionally also include heteroatoms within the meaning herein. Examples of acyl groups include, but are not limited to, a nicotinoyl group (pyridyl-3-carbonyl) acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “aldehyde” by itself or as part of another substituent refers to a chemical compound that has a —CHO group.

The term “alkene” or “alkenyl” by itself or as part of another substituent refers to either a straight chain, branched chain, or cyclic hydrocarbon, having at least one double bond between two carbon atoms. Examples of alkene groups include, but are not limited to, vinyl, propenyl, isopropenyl, 1-butenyl, 2-butenyl, isobutenyl, butadienyl, 1-pentenyl, 2-pentenyl, isopentenyl, 1,3-pentadienyl, 1,4-pentadienyl, 1-hexenyl, 2-hexenyl, 3-hexenyl, 1,3-hexadienyl, 1,4-hexadienyl, 1,5-hexadienyl, 2,4-hexadienyl, or 1,3,5-hexatrienyl. The alkene group is typically monovalent, but can be divalent, such as when the alkenyl group links two moieties together.

The term “alkoxy” by itself or as part of another substituent refers to an alkyl group, as defined above, having an oxygen atom that connects the alkyl group to the point of attachment. Alkoxy groups include, for example, methoxy, ethoxy, propoxy, iso-propoxy, butoxy, 2-butoxy, iso-butoxy, sec-butoxy, tert-butoxy, pentoxy, hexoxy, etc. The alkoxy groups can be further substituted with a variety of substituents described within. For example, the alkoxy groups can be substituted with halogens to form a “halo-alkoxy” group.

The term “alkyl” by itself or as part of another substituent refers to a straight or branched, saturated, aliphatic radical having the number of carbon atoms indicated. Alkyl groups can be optionally substituted alkyl groups. For example, C₁-C₆ alkyl includes, but is not limited to, methyl, ethyl, propyl, isopropyl, butyl, isobutyl, sec-butyl, tert-butyl, pentyl, isopentyl, hexyl, etc. Other alkyl groups include, but are not limited to heptyl, octyl, nonyl, decyl, etc. Alkyl can include any number of carbons, such as 1-2, 1-3, 1-4, 1-5, 1-6, 1-7, 1-8, 1-9, 1-10, 2-3, 2-4, 2-5, 2-6, 3-4, 3-5, 3-6, 4-5, 4-6 and 5-6. The alkyl group is typically monovalent, but can be divalent, such as when the alkyl group links two moieties together.

The term “alkyne” or “alkynyl” by itself or as part of another substituent refers to either a straight chain or branched hydrocarbon, having at least one triple bond between two carbon atoms. Examples of alkynyl groups include, but are not limited to, acetylenyl, propynyl, 1-butynyl, 2-butynyl, isobutynyl, sec-butynyl, butadiynyl, 1-pentynyl, 2-pentynyl, isopentynyl, 1,3-pentadiynyl, 1,4-pentadiynyl, 1-hexynyl, 2-hexynyl, 3-hexynyl, 1,3-hexadiynyl, 1,4-hexadiynyl, 1,5-hexadiynyl, 2,4-hexadiynyl, or 1,3,5-hexatriynyl. The alkynyl group is typically monovalent, but can be divalent, such as when the alkynyl group links two moieties together.

The term “Analyte” refers to a molecule, compound, or other component in a sample. Analytes may include but are not limited to peptides, proteins, polynucleotides, organic molecules, sugars and other carbohydrates, and lipids.

The term “amine” by itself or as part of another substituent as used herein refers to an alkyl groups as defined within, having one or more amino groups. The amino groups can be primary, secondary or tertiary. The alkyl amine can be further substituted with a hydroxy group. Amines useful in the present disclosure include, but are not limited to, ethyl amine, propyl amine, isopropyl amine, ethylene diamine and ethanolamine. The amino group can link the alkyl amine to the point of attachment with the rest of the compound, be at the omega position of the alkyl group, or link together at least two carbon atoms of the alkyl group. One of skill in the art will appreciate that other alkyl amines are useful in the present disclosure.

The term “amino group” refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The term “amide” refers to a functional group having a carbonyl group attached to an amine group, having the general formula RC(═O)NR′R″, where R, R′, and R″ represent organic groups or hydrogen atoms. The term “amido” refers to a substituent containing an amide group.

The term “ammonium” by itself or as part of another substituent refers to a cation having the formula NHR₃⁺ where each R group, independently, is hydrogen or a substituted or unsubstituted alkyl, aryl, aralkyl, or alkoxy group. Preferably, each of the R groups is hydrogen.

The term “Antibody” refers to an immunoglobulin protein or to a fragment or derivative thereof which specifically binds to an analyte. Antibodies include various classes and isotypes of immunoglobulins, such as IgA, IgD, IgE, IgG1, IgG2a, IgG2b, IgG3, and IgM. Antibody fragments include molecules such as Fab, scFv, F(ab′)2, and Fab′ molecules. Antibody derivatives include antibodies or fragments thereof having additions or substitutions, such as chimeric antibodies. Antibodies can be derived from human or animal sources, from hybridomas, through recombinant methods, or in any other way known to the art.

The term “aralkyl” refers to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The term “aryl” by itself or as part of another substituent refers to cyclic aromatic hydrocarbon groups that do not contain heteroatoms in the aromatic ring assembly. “Aryl” groups can be a monocyclic or fused bicyclic, tricyclic or greater, aromatic ring assembly containing 6 to 16 ring carbon atoms. For example, aryl may be, but is not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, benzyl or naphthyl. “Arylene” means a divalent radical derived from an aryl group. Aryl groups can be mono-, di- or tri-substituted by one, two or three radicals selected from alkyl, alkoxy, aryl, hydroxy, halogen, cyano, amino, amino-alkyl, trifluoromethyl, alkylenedioxy and oxy-C₂-C₃-alkylene; all of which are optionally further substituted, for instance as hereinbefore defined; or 1- or 2-naphthyl; or 1- or 2-phenanthrenyl. Alkylenedioxy is a divalent substitute attached to two adjacent carbon atoms of phenyl, e.g. methylenedioxy or ethylenedioxy. Oxy-C₂-C₃-alkylene is also a divalent substituent attached to two adjacent carbon atoms of phenyl, e.g. oxyethylene or oxypropylene. An example for oxy-C₂-C₃-alkylene-phenyl is 2,3-dihydrobenzofuran-5-yl.

The term “aryloxy” by itself or as part of another substituent refers to a O-aryl group, wherein aryl is as defined above. An aryloxy group can be unsubstituted or substituted with one or two suitable substituents. The term “phenoxy” refers to an aryloxy group wherein the aryl moiety is a phenyl ring. The term “(hetero)aryloxy” as used herein means an —O-heteroaryl group, wherein heteroaryl is as defined below. The term “(hetero)aryloxy” is used to indicate the moiety is either an aryloxy or (hetero)aryloxy group.

The term “heteroaryl” by itself or as part of another substituent refers to a monocyclic or fused bicyclic or tricyclic aromatic ring assembly containing 5 to 16 ring atoms, where from 1 to 4 of the ring atoms are a heteroatom each N, O or S. For example, heteroaryl includes pyridyl, indolyl, indazolyl, quinoxalinyl, quinolinyl, isoquinolinyl, benzothienyl, benzofuranyl, furanyl, pyrrolyl, thiazolyl, benzothiazolyl, oxazolyl, isoxazolyl, triazolyl, tetrazolyl, pyrazolyl, imidazolyl, thienyl, or any other radicals substituted, especially mono- or di-substituted, by e.g. alkyl, nitro or halogen. Pyridyl represents 2-, 3- or 4-pyridyl, advantageously 2- or 3-pyridyl. Thienyl represents 2- or 3-thienyl. Quinolinyl represents preferably 2-, 3- or 4-quinolinyl. Isoquinolinyl represents preferably 1-, 3- or 4-isoquinolinyl. Benzopyranyl, benzothiopyranyl represents preferably 3-benzopyranyl or 3-benzothiopyranyl, respectively. Thiazolyl represents preferably 2- or 4-thiazolyl, and most preferred, 4-thiazolyl. Triazolyl is preferably 1-, 2- or 5-(1,2,4-triazolyl). Tetrazolyl is preferably 5-tetrazolyl.

In some embodiments, heteroaryl is pyridyl, indolyl, quinolinyl, pyrrolyl, thiazolyl, isoxazolyl, triazolyl, tetrazolyl, pyrazolyl, imidazolyl, thienyl, furanyl, benzothiazolyl, benzofuranyl, isoquinolinyl, benzothienyl, oxazolyl, indazolyl, or any of the radicals substituted, especially mono- or di-substituted.

In some embodiments, substituents for the aryl and heteroaryl groups are varied and are selected from: -halogen, —OR′, —OC(O)R′, —NR′R″, —SR′, —R′, —CN, —NO₂, —CO₂R′, —CONR′R″, —C(O)R′, —OC(O)NR′R″, —NR″C(O)R′, —NR″C(O)₂R′, —NR′—C(O)NR″R″, —NH—C(NH₂)═NH, —NR′C(NH₂)═NH, —NH—C(NH₂)═NR′, —S(O)R′, —S(O)₂R′, —S(O)₂NR′R″, —N₃, —CH(Ph)₂, perfluoro (C₁-C₄)alkoxy, and perfluoro (C₁-C₄)alkyl, in a number ranging from zero to the total number of open valences on the aromatic ring system; and where R′, R″ and R′″ are independently selected from hydrogen, (C₁-C₅)alkyl and heteroalkyl, unsubstituted aryl and heteroaryl, (unsubstituted aryl)-(C₁-C₄)alkyl, and (unsubstituted aryl)oxy-(C₁-C₄)alkyl.

Two of the substituents on adjacent atoms of the aryl or heteroaryl ring may optionally be replaced with a substituent of the formula -T-C(O)—(CH₂)_{q}—U—, wherein T and U are independently —NH—, —O—, —CH₂— or a single bond, and q is an integer of from 0 to 2. Alternatively, two of the substituents on adjacent atoms of the aryl or heteroaryl ring may optionally be replaced with a substituent of the formula -A-(CH₂)ᵣ—B—, wherein A and B are independently —CH₂—, —O—, —NH—, —S—, —S(O)—, —S(O)₂—, —S(O)₂NR′— or a single bond, and r is an integer of from 1 to 3. One of the single bonds of the new ring so formed may optionally be replaced with a double bond. Alternatively, two of the substituents on adjacent atoms of the aryl or heteroaryl ring may optionally be replaced with a substituent of the formula —(CH₂)ₛ—X—(CH₂)ₜ—, where s and t are independently integers of from 0 to 3, and X is —O—, —NR′—, —S—, —S(O)—, —S(O)₂—, or —S(O)₂NR′—. The substituent R′ in-NR′—and —S(O)₂NR′— is selected from hydrogen or unsubstituted (C₁-C₆)alkyl.

As used herein, the term “azide” by itself or as part of another substituent refers to a structure-N₃.

The term “Binding partner” refers to a molecule capable of specifically binding an analyte. A binding partner can be any of a number of different types of molecules, including an antibody or antigen-binding fragment thereof, or other protein, peptide, polysaccharide, lipid, a nucleic acid or nucleic-acid analog, such as an oligonucleotide or PNA (peptide nucleic acids).

The term “boronic acid” by itself or as part of another substituent refers to a structure —B(OH)₂. It is recognized by those skilled in the art that a boronic acid may be present as a boronate ester at various stages in the synthesis of the quenchers. Boronic acid is meant to include such esters. The term “boronic ester” or “boronate ester” as used herein refers to a chemical compound containing a —B(Z¹)(Z²) moiety, wherein Z¹ and Z² together form a moiety where the atom attached to boron in each case is an oxygen atom. In some embodiments, the boronic ester moiety is a 5-membered ring. In some other embodiments, the boronic ester moiety is a 6-membered ring. In some other embodiments, the boronic ester moiety is a mixture of a 5-membered ring and a 6-membered ring.

The term “carbamate” by itself or as part of another substituent refers to the functional group having the structure-NR″CO₂R′, where R′ and R″ are independently selected from hydrogen, (C₁-C₅)alkyl and heteroalkyl, unsubstituted aryl and heteroaryl, (unsubstituted aryl)-(C₁-C₄)alkyl, and (unsubstituted aryl)oxy-(C₁-C₄)alkyl. Examples of carbamates include t-Boc, Fmoc, benzyloxy-carbonyl, alloc, methyl carbamate, ethyl carbamate, 9-(2-sulfo) fluorenylmethyl carbamate, 9-(2,7-dibromo) fluorenylmethyl carbamate, Tbfmoc, Climoc, Bimoc, DBD-Tmoc, Bsmoc, Troc, Teoc, 2-phenylethyl carbamate, Adpoc, 2-chloroethyl carbamate, 1,1-dimethyl-2-haloethyl carbamate, DB-t-BOC, TCBOC, Bpoc, t-Bumeoc, Pyoc, Bnpeoc, V-(2-pivaloylamino)-1,1-dimethylethyl carbamate, NpSSPeoc.

The term “carboxylic acid” by itself or as part of another substituent refers to a structure R—COOH where R is a carbon-containing group of atoms.

The term “carboxylate” by itself or as part of another substituent refers to the conjugate base of a carboxylic acid, which generally can be represented by the formula RCOO⁻. For example, the term “magnesium carboxylate” refers to the magnesium salt of the carboxylic acid The term “carboxylate ester” as used herein by itself or as part of another substituent refers to a compound derived from a carboxylic acid, which generally can be represented by the formula RCOOR′ where R′ can be an alkyl, alkene, alkyne, haloalkyl, heteroalkyl, cycloalkyl, aryl, heteroaryl, (unsubstituted aryl)alkyl, and (unsubstituted aryl)oxy-alkyl or other carbon-containing group of atoms. R′ can optionally contain functional groups.

The term “CD” refers to Cluster of differentiation.

The term “chromophore” refers to a compound having a reactive group (e.g., a carboxylate moiety, an amino moiety, a haloalkyl moiety, or the like) that can be covalently bonded. Examples of suitable chromophores include, but are not limited to, those described in U.S. Pat. Nos. 7,687,282; 7,671,214; 7,446,202; 6,972,326; 6,716,979; 6,579,718; 6,562,632; 6,399,392; 6,316,267; 6,162,931; 6,130,101; 6,005,113; 6,004,536; 5,863,753; 5,846,737; 5,798,276; 5,723,218; 5,696,157; 5,658,751; 5,656,449; 5,582,977; 5,576,424; 5,573,909; and 5,187,288, which patents are incorporated herein by reference in their entirety.

The term “Compensation” in flow cytometry is a mathematical process of correcting for fluorescence spillover (spectral overlap of multiparameter flow cytometric data). For example, compensation may be performed by removing the signal of any given fluorochrome from all detectors except the one devoted to measuring that dye. Since fluorochromes may have wide-ranging spectrum, they can overlap, causing the undesirable confusion during data analysis.

The term “cycloalkyl” by itself or as part of another substituent refers to a saturated or partially unsaturated, monocyclic, fused bicyclic or bridged polycyclic ring assembly containing from 3 to 12 ring atoms, or the number of atoms indicated monocyclic rings include, for example, cyclopropyl, cyclobutyl, cyclopentyl, cyclohexyl, and cyclooctyl. Bicyclic and polycyclic rings include, for example, norbornane, decahydronaphthalene and adamantane. For example, C₃₋₈ cycloalkyl includes cyclopropyl, cyclobutyl, cyclopentyl, cyclohexyl, cyclooctyl, and norbornane.

The term “diazonium salt” by itself or as part of another substituent refers to a group of organic compounds with a structure of R—N₂⁺X⁻, wherein R can be any organic group (e.g., alkyl or aryl) and X is an inorganic or organic anion (e.g., halogen).

The term “DM-2” or “DM2” refers to DM-2. The term “S” refers to stabilizer in reference to “DM2+S” buffer. “DM2+S” is a DM buffer according to the disclosure, wherein the carbohydrate stabilizer is trehalose or a hydrate thereof, the water-soluble monomer is monomer A, the protein stabilizer is a casein, and the zwitterionic surfactant is DMMA, for example, according to Table 3.

The term “dye conjugate” refers to a dye conjugated to a binding partner.

The term “fluorescent dye” refers to a dye comprising a light excitable fluorophore that can re-emit light upon light excitation. The term “Fluorescent Dye” encompasses both fluorescent polymeric dyes and fluorescent non-polymeric dyes, including fluorescent monomeric and other traditional fluorescent dyes. For example, SuperNova™ (“SN”) v428 (Beckman Coulter, Inc.) is a fluorescent polymer dye optimally excited by the violet laser (405 nm) with an excitation maximum of 414 nm, an emission peak of 428 nm, and can be detected using a 450/50 bandpass filter or equivalent. SN v605 and SN v786 are tandem polymer dyes, derived from the core SN v428 polymer dye. Both share the same absorbance characteristics, with maximum excitation at 414 nm. With emission peaks for SN v605 and SN v786 at 605 nm and 786 nm respectively, they are optimally detected using the 610/2 and 780/60 nm bandpass filters of the flow cytometer.

The term “fluorophore” refers to a fluorescent chemical compound that can re-emit light upon light excitation. Fluorophores may typically contain several combined aromatic groups, or planar and cyclic molecules with several π pi bonds.

The term “halogen” by itself or as part of another substituent refers to fluorine, chlorine, bromine and iodine.

The term “(hetero)arylamino” by itself or as part of another substituent refers an amine radical substituted with an aryl group (e.g., —NH-aryl). An arylamino may also be an aryl radical substituted with an amine group (e.g., -aryl-NH₂). Arylaminos may be substituted or unsubstituted.

As used herein, the term “hydrazone” by itself or as part of another substituent refers to a structure

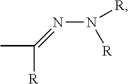

where R can be, for example, a water solubilizing moiety, hydrogen, alkyl, alkene, alkyne, cycloalkyl, haloalkyl, aryl, or other and can contain carboxylic groups. R can be a water-solubilizing polymer including, but not limited to, a polymer comprising 6 or more monomeric units, a non-ionic water-soluble polymer, PEG, modified PEG terminated with a carboxylic acid or a carboxylic ester.

The terms “hydrazine” and “hydrazide” by themselves or as part of another substituent refer to compounds that contain singly bonded nitrogens, one of which is a primary amine functional group.

The term “hydrocarbon” or “hydrocarbyl” refers to a molecule or functional group that includes carbon and hydrogen atoms. The term can also refer to a molecule or functional group that normally includes both carbon and hydrogen atoms but wherein some or all the hydrogen atoms are substituted with other functional groups. The term “hydrocarbyl” refers to a functional group derived from a straight chain, branched, or cyclic hydrocarbon, and can be alkyl, alkenyl, alkynyl, aryl, cycloalkyl, acyl, or any combination thereof. Hydrocarbyl groups can be shown as (Cₐ-C_{b}) hydrocarbyl, wherein a and b are integers and mean having any of a to b number of carbon atoms. For example, (C₁-C₄) hydrocarbyl means the hydrocarbyl group can be methyl (C₁), ethyl (C₂), propyl (C₃), or butyl (C₄), and (C₀-C_{b}) hydrocarbyl means in certain embodiments there is no hydrocarbyl group. A hydrocarbylene group is a diradical hydrocarbon, e.g., a hydrocarbon that is bonded at two locations.

The term “Labeled binding partner” refers to a binding partner that is conjugated to a dye.

The term “linker” or “linkage” refers to a linking moiety that connects two groups and has a backbone of 100 atoms or less in length. A linker or linkage may be a covalent bond that connects two groups or a chain of between 1 and 100 atoms in length, for example a chain of 1, 2, 3, 4, 5, 6, 8, 10, 12, 14, 16, 18, 20 or more carbon atoms in length, where the linker may be linear, branched, cyclic or a single atom. In some embodiments, the linker is a branching linker that refers to a linking moiety that connects three or more groups. In certain cases, one, two, three, four or five or more carbon atoms of a linker backbone may be optionally substituted with a sulfur, nitrogen or oxygen heteroatom. In some embodiments, the linker backbone includes a linking functional group, such as an ether, thioether, amino, amide, sulfonamide, carbamate, thiocarbamate, urea, thiourea, ester, thioester or imine. The bonds between backbone atoms may be saturated or unsaturated, and in some cases not more than one, two, or three unsaturated bonds are present in a linker backbone. The linker may include one or more substituent groups, for example with an alkyl, aryl or alkenyl group. A linker may include, without limitations, polyethylene glycol, ethers, thioethers, tertiary amines, alkyls, which may be straight or branched, e.g., methyl, ethyl, n-propyl, 1-methylethyl (iso-propyl), n-butyl, n-pentyl, 1,1-dimethylethyl (t-butyl), and the like. The linker backbone may include a cyclic group, for example, an aryl, a heterocycle or a cycloalkyl group, where 2 or more atoms, e.g., 2, 3 or 4 atoms, of the cyclic group are included in the backbone. A linker may be cleavable or non-cleavable.

A linker moiety can be attached to “L” or to “A”, as taught in US Published Application No. 2020/0190253A1, which is incorporated herein by reference in its entirety. A linker moiety can comprise a sulfonamide, disulfonamide, a selenomide, a sulfinamide, a sultam, a disulfinamide, an amide, a seleninamide, a phosphonamide, a phosphinamide, a phosphonamidate, or a secondary amine.

As described therein, and as each pertains to a linker moiety, the term “sulfonamide,” refers to a moiety —S(O)₂NR—; the term “disulfonamide,” refers to a moiety —S(O)₂NRS(O)₂—; the term “selenonamide,” refers to a moiety —Se(O)₂NR—; the term “sulfinamide,” refers to a moiety —S(O)NR—; the term “disulfinamide,” refers to a moiety —S(O)NRS(O)—; the term “seleninamide,” refers to a moiety —Se(O)NR—; the term “phosphonamide,” refers to a moiety —NR—PR(O)NR—; the term “phosphinamide,” refers to a moiety —PR(O)NR—; and the term “phosphonamidate,” refers to a moiety —O—PR(O)NR—; and the term “sultam” refers to a cyclic sulfonamide (e.g., wherein the R group is bonded to the sulfur atom via an alkylene moiety); wherein for each term the R group is independently H, alkyl, haloalkyl, or aryl.

As used herein, the term “N-hydroxysuccinimidyl” by itself or as part of another substituent refers to a structure

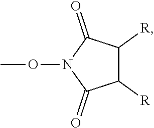

where R can be, for example, a water solubilizing moiety, hydrogen, alkyl, alkene, alkyne, cycloalkyl, haloalkyl, aryl, or other and can contain carboxylic groups. R can be a water-solubilizing polymer including, but not limited to, a polymer comprising 6 or more monomeric units, a non-ionic water-soluble polymer, PEG, modified PEG terminated with a carboxylic acid or a carboxylic ester.

The term “Reactant solution” refers to solution comprising the labeled binding partner. In some embodiments, besides the labeled binding partner, a reactant solution further comprises stabilizers, salt, buffer, surfactants, and/or other reagents. The term “maleimide” by itself or as part of another substituent refers a structure

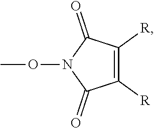

where R can be, for example, a water solubilizing moiety, hydrogen, alkyl, alkene, alkyne, cycloalkyl, haloalkyl, aryl, or other and can contain carboxylic groups. R can be a water-solubilizing polymer including, but not limited to, a polymer comprising 6 or more monomeric units, a non-ionic water-soluble polymer, PEG, modified PEG terminated with a carboxylic acid or a carboxylic ester.

The term “MdFl” or “MDFl” refers to Median fluorescent intensity.

The term “% recruitment” refers to number of gated cells of relevant population.

The term “Multi-Color antibody panel” refers to a cocktail comprising a plurality of different fluorescent dye conjugates (e.g., CD4-FITC, CD8-PE, CD20-APC, CD3-PC5.5, CD16-FITC, CD25-PE, CD3-ECD, CD38-PC5.5, CD27-PC7, CD10-APC, CD14-APCA700, CD45-AA750, CD8-KRO, CD56-SNv428, CD20-SNv605, CD4-SNv786, etc.) in a liquid or dried format that may be used directly to stain blood and analyze it in a flow cytometer.

The term “multi-color dry reagent” refers to a cocktail of different fluorescent dye conjugates (CD4-FITC, CD8-PE, CD20-APC, CD3-PC5.5, etc.) in a dried format that may be used directly to stain blood and analyze it in a flow cytometer. A multi-color dry reagent cocktail having only conventional dyes such as FITC, PE, ECD, PC5, PC5.5, PC7, APC, AA700, AA750, PBE and KrO can be dried using prior drying technology. However, prior drying technology was found to be ineffective while drying multiple fluorescent polymer dye antibody conjugates in a cocktail. These polymer dye conjugates tend to non-specifically interact and lead to the difficulties in resolving the population, which might lead to challenges in identification of the desired cell populations in a given sample.

The term “Multiplexing” herein refers to an assay or other analytical method in which multiple analytes can be assayed simultaneously.

The term “oligoether” refers to an oligomer containing structural repeat units having an ether functionality. As used herein, an “oligomer” is understood to mean a molecule that contains one or more identifiable structural repeat units of the same or different formula.

The term “organic group” refers to any carbon-containing functional group. Examples of carbon-containing functional groups can include an oxygen-containing group such as an alkoxy group, aryloxy group, aralkyloxy group, oxo (carbonyl) group; a carboxyl group including a carboxylic acid, carboxylate, and a carboxylate ester; a sulfur-containing group such as an alkyl and aryl sulfide group; and other heteroatom-containing groups. Non-limiting examples of organic groups include OR, OOR, OC(O)N(R)₂, CN, CF₃, OCF₃, R, C(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH) N(R)₂, C(O)N(OR)R, C(═NOR)R, and substituted or unsubstituted (C₁-C₁₀₀) hydrocarbyl, wherein R can be hydrogen (in examples that include other carbon atoms) or a carbon-based moiety, and wherein the carbon-based moiety can be substituted or unsubstituted.

The term “PEG” refers to polyethylene glycol, or poly(ethylene glycol), the family of biocompatible water-solubilizing linear polymers based on the ethylene glycol monomer unit described by the formula —(CH₂—CH₂—O—)ₙ— or a derivative thereof. In some embodiments, “n” is 1000 or less, 500 or less, 200 or less, 100 or less, 50 or less, 40 or less, 30 or less, 20 or less, 15 or less, such as 3 to 15, or 10 to 15. It is understood that the PEG polymeric group may be of any convenient length and may include a variety of terminal groups and/or further substituent groups, including but not limited to, alkyl, aryl, hydroxyl, amino, acyl, carboxylic acid, carboxylate ester, acyloxy, and amido terminal and/or substituent groups. The number after “PEG” refers to the average molecular weight, where Mw refers to weight average molecular weight, and Mn refers to number average molecular weight.

The term “PBS” refers to phosphate buffered saline which is an aqueous buffer which may contain sodium chloride, disodium hydrogen phosphate, potassium chloride, and potassium dihydrogen phosphate. For example, PBS may contain milliQ water or deionized water and 137 mM NaCl, 2.7 mM KCl, 10 mM Na₂HPO₄, 1.8 mM KH₂PO₄. The pH may be about pH 7.0-7.4. The PBS may or may not be preserved with an azide such as sodium azide. PBS is an isotonic solution.

The term “phosphoramide” by itself or as part of another substituent refers to a structure

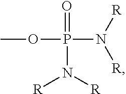

where R can be, for example, a water solubilizing moiety, hydrogen, alkyl, alkene, alkyne, cycloalkyl, haloalkyl, aryl, or other and can contain carboxylic groups. R can be a water-solubilizing polymer including, but not limited to, a polymer comprising 6 or more monomeric units, a non-ionic water-soluble polymer, PEG, modified PEG terminated with a carboxylic acid or a carboxylic ester.

The term “phosphonamidate” by itself or as part of another substituent refers to a structure

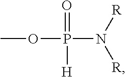

where R can be, for example, a water solubilizing moiety, hydrogen, alkyl, alkene, alkyne, cycloalkyl, haloalkyl, aryl, or other and can contain carboxylic groups. R can be a water-solubilizing polymer including, but not limited to, a polymer comprising 6 or more monomeric units, a non-ionic water-soluble polymer, PEG, modified PEG terminated with a carboxylic acid or a carboxylic ester.

The term “phosphinamide” by itself or as part of another substituent refers to a structure

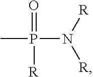

where R can be, for example, a water solubilizing moiety, hydrogen, alkyl, alkene, alkyne, cycloalkyl, haloalkyl, aryl, or other and can contain carboxylic groups. R can be a water-solubilizing polymer including, but not limited to, a polymer comprising 6 or more monomeric units, a non-ionic water-soluble polymer, PEG, modified PEG terminated with a carboxylic acid or a carboxylic ester.

The term “Physical Properties” refers to properties including brightness or fluorescence of the fluorescent dye conjugate and its spillover into other channels.

The term “polymer dye conjugate” refers to a polymer dye conjugated to a binding partner. For example, a polymer dye conjugate can comprise fluorescent polymers having monomer subunits including, but not limited to, dihydrophenanthrene (DHP), fluorene, and combinations thereof.

The term “substantially reduced” refers to at least 10%, at least 25%, or at least 50% reduction of a measurable quality.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo (carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH) N(R)₂, C(O)N(OR)R, and C(═NOR)R, wherein R can be hydrogen or a carbon-based moiety; for example, R can be hydrogen, (C₁-C₁₀₀) hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

The term “sulfonate functional group” or “sulfonate” either by itself or as part of another substituent refers to both the free sulfonate anion (—S(═O)₂O—) and salts thereof. Therefore, the term sulfonate encompasses sulfonate salts such as sodium, lithium, potassium and ammonium sulfonate.

The term “sulfonamido” by itself or as part of another substituent refers to a group of formula —SO₂NR— where R can be, for example, a water solubilizing moiety, hydrogen, alkyl, alkene, alkyne, cycloalkyl, haloalkyl, aryl, or other and can contain carboxylic groups. R can be a water-solubilizing polymer including, but not limited to, a polymer comprising 6 or more monomeric units, a non-ionic water-soluble polymer, PEG, modified PEG terminated with a carboxylic acid or a carboxylic ester.

The term “sulfonamide” by itself or as part of another substituent refers to a group of formula —SO₂NR₂ where R can be, for example, a water solubilizing moiety, hydrogen, alkyl, alkene, alkyne, cycloalkyl, haloalkyl, aryl, or other and can contain carboxylic groups. R can be a water-solubilizing polymer including, but not limited to, a polymer comprising 6 or more monomeric units, a non-ionic water-soluble polymer, PEG, modified PEG terminated with a carboxylic acid or a carboxylic ester.

The term “sulfinamide” by itself or as part of another substituent refers to a group of formula —SONR₂— where R can be, for example, a water solubilizing moiety, hydrogen, alkyl, alkene, alkyne, cycloalkyl, haloalkyl, aryl, or other and can contain carboxylic groups. R can be a water-solubilizing polymer including, but not limited to, a polymer comprising 6 or more monomeric units, a non-ionic water-soluble polymer, PEG, modified PEG terminated with a carboxylic acid or a carboxylic ester.

The term “silyl” by itself or as part of another substituent refers to Si(R^{z})₃ wherein each R^{z} independently is alkyl, aryl or other carbon-containing group of atoms.

The term “thiol” by itself or as part of another substituent refers to a compound that contains the functional group composed of a sulfur-hydrogen bond. The general chemical structure of the thiol functional group is R—SH, where R represents an alkyl, alkene, aryl, or other carbon-containing group of atoms.

The term “water-solubilizing moiety” as used herein by itself or part of another group refers to any hydrophilic group that is well solvated in aqueous environments, for example such as under physiological conditions, and is capable of increasing the water solubility of the molecule to which it is attached. The increase in water solubility of the molecule can vary depending upon the moiety attached. In some instances, the increase in water solubility (as compared to the solubility of the molecule without the moiety attached) is 2 fold or more, 5 fold or more, 10 fold or more, 25 fold or more, 50 fold or more, or 100 fold or more. “Water-solubilizing moiety” includes moieties, such as, but not limited to, PEG groups, carboxy groups including but not limited to carboxylic acids and carboxylates, polyvinyl alcohol, glycols, peptides, polyphosphates, polyalcohols, sulfonates, phosphonates, boronates, amines, ammoniums, sulfoniums, phosphonium, alcohols, zwitterionic derivatives, carbohydrates, nucleotides, polynucleotides, substituted PEG groups, substituted carboxy groups including but not limited to substituted carboxylic acids and substituted carboxylates, substituted glycols, substituted peptides, substituted polyphosphates, substituted polyalcohols, substituted sulfonates, substituted phosphonates, substituted boronates, substituted amines, substituted ammoniums, substituted sulfoniums, substituted phosphonium, alcohols, substituted zwitterionic derivatives, substituted carbohydrates, substituted nucleotides, substituted polynucleotides, and combinations thereof.

The term “water-soluble polymer” (WSP) as used herein refers to a polymer having solubility in “water” as used herein of 1 mg/mL or more, such as 3 mg/mL or more, 10 mg/mL or more, 20 mg/mL or more, 30 mg/mL or more, 40 mg/mL or more, 50 mg/mL or more, 60 mg/mL or more, 70 mg/mL or more, 80 mg/mL or more, 90 mg/mL or more, 100 mg/mL or more, or even more. It is understood that water soluble polymers may, under certain conditions, form discrete water-solvated nanoparticles in aqueous systems and can be resistant to aggregation.

### Reaction Vessel

A “reaction vessel” as disclosed herein can be any container where reactions between the binding partners or dye conjugates thereof and the target analytes can occur. For example, a reaction vessel can be a tube, a plate, a well of a microtiter plate, a chamber, and a slide. In a preferred embodiment, a reaction vessel has a lid or cap such that the binding reaction can occur in a closed environment.

### Substrate

A reaction vessel comprises one or more substrates. The “substrate” can be any suitable surface, including but not limited to, plastic, nitrocellulose, cellulose acetate, quartz, and glass. Non-limiting examples of plastic may include polystyrene, polypropylene, cyclo-olefin, and polycarbonate. In some embodiments, the substrate is a membrane. The substrate can be the inside surface of the body of a reaction vessel, e.g., a plastic tube or well of a microtiter plate. The substrate can also be a bead. In some embodiments, at least one of the substrates receiving the labeled binding partners (e.g., a membrane) is bonded to an inside surface of the body of the reaction vessel. In some embodiments, the membrane substrate is a sheet or roll, which makes it easier to deposit the solutions and easier to dry. In some embodiments, the membrane can be cut to separate individual dried reactant spots. In some embodiments, the cut membrane is simply dropped into the reaction vessel. In some preferred embodiments, the cut membranes are bonded to the surface of the reaction vessel, so that the membrane do not escape the vessel when liquid is pipetting into or out of the reaction vessel.

The acronym “SN” refers to SuperNova™.

The acronym “SSC” refers to side scatter.

The term “WBC” refers to white blood cells.

### Liquid Sample

The reaction vessel is configured to receive a liquid sample. Liquid samples used in the invention typically comprise target analytes obtained as or dispersed in a predominantly aqueous medium. Samples can be any source of biological material, such as proteins, carbohydrates, or polynucleotides that can be obtained from a living organism, directly or indirectly. Samples can include, e.g., cells, tissue or fluid, and the deposits left by that organism, including viruses, mycoplasma, and fossils. The sample may comprise a target analyte prepared through synthetic means, in whole or in part. Non-limiting examples of the sample include blood, serum, plasma, urine, semen, milk, sputum, mucus, a buccal swab, a vaginal swab, a rectal swab, an aspirate, a needle biopsy, a section of tissue obtained for example by surgery or autopsy, plasma, serum, spinal fluid, lymph fluid, the external secretions of the skin, respiratory, intestinal, and genitourinary tracts, tears, saliva, tumors, organs, samples of in vitro cell culture constituents (including but not limited to conditioned medium resulting from the growth of cells in cell culture medium, putatively virally infected cells, recombinant cells, and cell components).

### Target Analytes

The present invention is designed to detect the presence, and in some cases the quantity of specific target analytes. The term “target analyte” refers to a target molecule to be detected in a biological sample, for example, peptides, proteins, polynucleotides, organic molecules, sugars and other carbohydrates, and lipids. It is an important aspect of the invention that the target analytes are comprised in a liquid sample and are accessible, or made accessible at some point, to bind analyte-specific binding partners of the instant invention. Target analytes may be found in a biological sample, such as a blood sample, a cell line development sample, a tissue culture sample, and the like.

Target analytes can be present on beads, or present and accessible on the surface of cells. Illustrative examples of useful analytes include, but are not limited to, the following: 1) specific cell surface macromolecules and antigens (including hormones, protein complexes, and molecules recognized by cell receptors) and 2) cellular proteins, DNA or RNA in permeabilized cells including abnormal DNA or RNA sequences or abnormal amounts of certain messenger RNA. Detection of these analytes is particularly useful in situations where they are contained in and/or are identifiers of rare cells such as are found in the early stages of a variety of cancers.

### Binding Partner

The term “binding partner” refers to a molecule that specifically binds to an epitope of a target analyte. A number of different types of binding partners can be used in the present system and methods. In one embodiment, the binding partner is an antibody. Antibodies used to bind a particular analyte are preferably monoclonal, and thus are directed against a single epitope of an analyte. Monoclonal antibodies can be prepared using various techniques known to the art, and are typically prepared through the creation of a hybridoma using a B-cell line that produces an antibody with desired binding characteristics. Antibodies directed against a single epitope can also be generated in other ways, such as through recombinant methods. In some embodiments, polyclonal antibodies can be used as specific binding partners in the present system and methods. For example, a binding partner can be polyclonal antibodies raised against epitopes of the analyte. Polyclonal antibodies can be prepared in ways known to the art, such as by immunizing a host and collecting plasma or serum from that host. Antibody fragments, which retain their specific binding characteristics, can also be used as specific binding partners in the present invention, including fragments lacking the Fc portion of an antibody, e.g., Fab, Fab′ and F(ab′)2 fragments. F(ab′)2 fragments can be produced by methods known to the art, e.g., by cleaving a monoclonal antibody with proteolytic enzymes such as papain and pepsin. Fab′ fragments can be produced by reductive cleavage of F(ab′)2 fragments with agents such as dithiothreitol or mercaptoethanol. Antibody fragments can alternatively be produced using recombinant methods, such as through the use of a phage display library.

Binding partners other than antibodies or antibody fragments or derivatives can also be used in the present system and methods. For example, binding partners may be nucleic acids or nucleic-acid analogs, such as oligonucleotides or PNA probes. In one embodiment, aptamers can be used as specific binding partners. Aptamers are single-stranded DNA or RNA (ssDNA or ssRNA) molecules that can bind to pre-selected targets including proteins and peptides with high affinity and specificity. Other binding partner that can bind to target analyte to form pairs of receptor-ligand, enzyme-substrate, enzyme-inhibitor, and enzyme-cofactor pairs can also be used. Specific examples of such binding partner pairs include carbohydrate and lectin, biotin and avidin or streptavidin, folic acid and folate binding protein, vitamin B12 and intrinsic factor, Protein A and immunoglobulin, and Protein G and immunoglobulin. Also included are binding partners that form a covalent bond with the target analytes.

### Dyes

A “dye” is a moiety that provides a detectable signal, which can be attached to or incorporated into a binding partner, either directly or indirectly. A dye used in the invention can be colored, fluorescent, or luminescent, and is typically detected by a detector in a flow cytometer, e.g., PMT or APD. Fluorescent dyes can be monomeric or polymeric. Non-limiting examples of monomeric dyes include fluorescein, rhodamine, and cyanine. For example, commonly used monomeric dye fluorochromes may include FITC (fluorescein isothiocyanate) (excitation max 494 nm/emission max 520 nm), PE (R-phycoerythrin) (excitation max 496 nm/emission max 578 nm), APC (allophycocyanin) (excitation max 650 nm/emission max 660 nm), and PerCP (carotenoid-protein complexes derived from phytoplankton) (excitation max 482 nm/emission max 678 nm), Cy5.5 (cyanine dye) (excitation max 675 nm/emission max 694 nm). Other cyanine dyes may be synthesized from 2-, 3-, 5-, or 7-methine structures and may include Cy2, Cy3, Cy3B, Cy3.5, Cy5, and Cy7.PC5.5. The fluorescent dye may be a tandem dye. Tandem dyes may include PE-Cy5.5 tandem (excitation max 566 nm/emission max 671 nm), APC-Cy5.5 tandem (excitation max 656 nm/emission max 700 nm), and PerCP-Cy5.5 tandems (excitation max 489 nm/emission max 679 nm).

The fluorescent dye may be a fluorescent polymer dye. Fluorescent polymeric dyes are particularly useful for analysis of chemical and biological targets. They are highly responsive optical reporters and efficient light absorbers, by virtue of the multiple chromophores they comprise. Examples of fluorescent polymeric dyes include, but are not limited to, conjugated polymers having repeat units of chromophore, aggregates of conjugated molecules, luminescent dyes attached via side chains to saturated polymers, semiconductor quantum dots and dendritic structures. Fluorescent polymeric and monomeric dyes disclosed in U.S. Pat. Nos. 7,214,489, 8,354,239, 8,575,303, can also be used for the present invention.

In some cases, the fluorescent dye may be a fluorescent polymer dye having a structure according to Formula (IV):

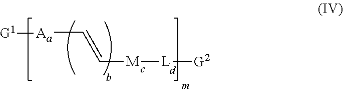

wherein:
each A is independently selected from the group consisting of an aromatic co-monomer and a heteroaromatic co-monomer;
each L is a linker moiety;
each M is independently selected from the group consisting of an aromatic co-monomer, a heteroaromatic co-monomer, a bandgap-modifying monomer, optionally substituted ethylene, and ethynylene;
G′ and G′ are independently selected from an unmodified polymer terminus and a modified polymer terminus;
a, c, and d independently define the mol % of each unit within the structure which each can be evenly or randomly repeated and where each a is a mol % from 10 to 100%, each c is a mol % from 0 to 90%, and each d is a mol % from 0 to 25%;
each b is independently 0 or 1; and
m is an integer from 1 to about 10,000.

L may be a linker moiety comprising a an aryl or heteroaryl group evenly or randomly distributed along the polymer main chain and may be substituted with one or more pendant chains terminated with a functional group selected from the group consisting of amine, carbamate, carboxylic acid, carboxylate, maleimide, activated ester, N-hydroxysuccinimidyl, hydrazine, hydrazide, hydrazone, azide, alkyne, aldehyde, thiol, and protected groups thereof for conjugation to another substrate, acceptor dye, molecule or binding agent.

A fluorescent polymer dye may be conjugated to a binding partner to form a fluorescent polymer dye conjugate, for example, having monomer A subunits and monomer B subunits, as described in, for example, US2020/0190253, which is incorporated herein by reference. A fluorescent polymer dye conjugate may have the structure of Formula V:

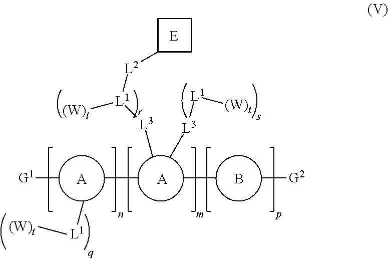

wherein:
each A is independently selected from the group consisting of an aromatic co-monomer and a heteroaromatic co-monomer;
L¹, L², and L³ are linker moieties;
W is a water-solubilizing moiety;
each E is an independently selected chromophore, functional moiety, or binding partner;
each B is independently selected from the group consisting of an aromatic co-monomer, a heteroaromatic co-monomer, a bandgap-modifying monomer, optionally substituted ethylene, and ethynylene;
G¹ and G² are independently selected from an unmodified polymer terminus and a modified polymer terminus;
subscripts n and m are independently integers ranging from 1 to 10,000, subscript p is an integer ranging from 0 to 10,000, and
the sum of subscripts n, m, and p ranges from 2 to 10,000;
subscript q is 1, 2, 3, or 4;
subscript r is 1, 2, 3, or 4;
subscript s is 0, 1, 2, or 3;
subscript t is 1 or 2
the sum of subscript r and s ranges from 1 to 4; and
A and B are distributed randomly or non-randomly in the conjugated polymer.

The fluorescent dye may be a fluorescent polymer dye having water-soluble monomer A subunits and monomer B subunits. The polymer dye may be a water-soluble fluorescent polymer dye. For example, the monomer A or monomer B may comprise a dihydrophenanthrene (DHP) moiety. The monomer A or monomer B may comprise a fluorene moiety. In some conjugated polymer dyes, a monomer B may be used to alter polymer band gap. The monomer units may be water-soluble, for example, comprising one or more, or two or more water-solubilizing moieties (W), such as poly(ethylene glycol) (PEG) moieties. In some embodiments, the monomer A or monomer or B each independently are a water-soluble monomer molecule. The water-soluble monomer A or monomer B may each independently comprise a DHP moiety and one or more or two or more PEG moieties. The water-soluble monomer A or monomer B may each independently comprise DHP moieties with solubilizing PEG moieties attached via sulfonamide groups.

The water-soluble monomer A or monomer B comprising a DHP moiety may each independently have a structure according to Formula (I):

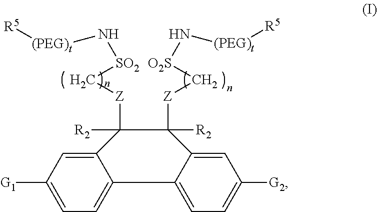

wherein
each G₁, G₂ is independently selected from the group consisting of hydrogen, alkyl, PEG, halogen, alkyne, optionally substituted aryl, optionally substituted heteroaryl, halogen substituted aryl, silyl, diazonium salt, triflate, acetyloxy, azide, sulfonate, phosphate, boronic acid substituted aryl, boronic ester substituted aryl, boronic ester, boronic acid, optionally substituted tetrahydropyrene (THP), optionally substituted fluorene, optionally substituted dihydrophenanthrene (DHP), aryl or heteroaryl substituted with one or more pendant chains terminated with a functional group selected from amine, carbamate, carboxylic acid, carboxylate, maleimide, activated ester, N-hydroxysuccinimidyl, hydrazine, hydrazide, hydrazone, azide, alkyne, aldehyde, thiol, and protected groups thereof;
each R² is independently selected from the group consisting of a water-solubilizing moiety, alkene, alkyne, cycloalkyl, haloalkyl, (hetero)aryloxy, (hetero)arylamino, sulfonamide-PEG, phosphoramide-PEG, ammonium alkyl salt, ammonium alkyloxy salt, ammonium oligoether salt, sulfonate alkyl salt, sulfonate alkoxy salt, sulfonate oligoether salt, sulfonamido oligoether, sulfonamide, sulfinamide, phosphonamidate, phosphinamide,

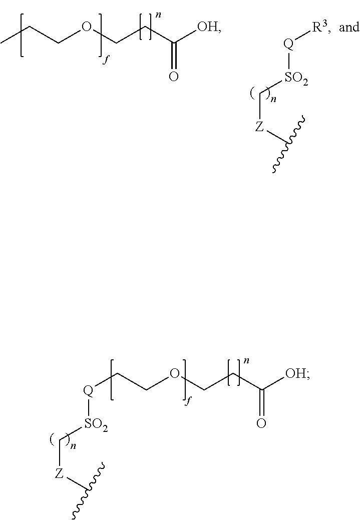

each R³ is a water-solubilizing moiety;
each R⁴ is independently selected from the group consisting of H, alkyl, PEG, a water-solubilizing moiety, a linker moiety, a chromophore, carboxylic amine, amine, carbamate, carboxylic acid, carboxylate ester, maleimide, activated ester, N-hydroxysuccinimidyl, hydrazine, hydrazide, hydrazone, azide, alkyne, aldehyde, or thiol, or protected groups thereof;
each R⁵ is independently selected from the group consisting of H, hydroxyl, C₁-C₁₂ alkyl, C₂-C₁₂ alkene, C₂-C₁₂ alkyne, C₃-C₁₂ cycloalkyl, C₁-C₁₂ haloalkyl, C₁-C₁₂ alkoxy, C₂-C₁₈ (hetero)aryloxy, C₂-C₁₈ (hetero)arylamino, C₂-C₁₂ carboxylic acid, C₂-C₁₂ carboxylate ester, and C₁-C₁₂ alkoxy;
each Q is independently a bond, NR₄, or —CH₂;
each Z is independently CH₂, O, or NR₄;
each f is independently an integer from 0 to 50; and
each n is independently an integer from 1 to 20.

In some embodiments, the water-soluble monomer A or monomer B comprising a DHP moiety may each independently have a chemical structure according to Formula (III):

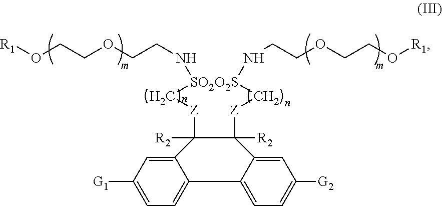

wherein each G₁, G₂ is independently halo (F, Cl, Br, I); each Z is independently selected from the group consisting of O, CH₂, and NH; each R₁ is independently alkyl (C₁-C₃); each R² is independently H, alkyl (C₁-C₆); each n is independently 1-6; and each m is independently 5-50. In some embodiments, G₁, G₂ are each Br; each Z is O;
each R₁ is CH₃; each R² is H; each n is independently 2-4; and each m is independently 5-20. In some embodiments, each n is 3; and each m is 11.

The water-soluble monomer A or monomer B may each independently comprise a fluorene moiety and one or more or two or more PEG moieties. The water-soluble monomer A or monomer B comprising a fluorene moiety may each independently have a structure according to Formula (II):

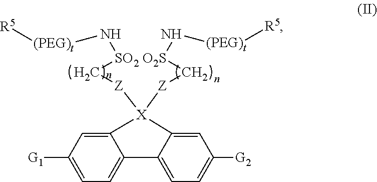

wherein
each G₁, G₂ is independently selected from the group consisting of hydrogen, halogen, alkyl, PEG, alkyne, optionally substituted aryl, optionally substituted heteroaryl, halogen substituted aryl, silyl, diazonium salt, triflate, acetyloxy, azide, sulfonate, phosphate, boronic acid substituted aryl, boronic ester substituted aryl, boronic ester, boronic acid, optionally substituted tetrahydropyrene (THP), optionally substituted fluorene, optionally substituted dihydrophenanthrene (DHP), aryl or heteroaryl substituted with one or more pendant chains terminated with a functional group selected from amine, carbamate, carboxylic acid, carboxylate, maleimide, activated ester, N-hydroxysuccinimidyl, hydrazine, hydrazide, hydrazone, azide, alkyne, aldehyde, thiol, and protected groups thereof;
each X is C or Si;
   each R⁴ is independently selected from the group consisting of H, alkyl, PEG, a water-solubilizing moiety, a linker moiety, a chromophore, carboxylic amine, amine, carbamate, carboxylic acid, carboxylate ester, maleimide, activated ester, N-hydroxysuccinimidyl, hydrazine, hydrazide, hydrazone, azide, alkyne, aldehyde, or thiol, or protected groups thereof;
each R⁵ is independently selected from the group consisting of H, hydroxyl, C₁-C₁₂ alkyl, C₂-C₁₂ alkene, C₂-C₁₂ alkyne, C₃-C₁₂ cycloalkyl, C₁-C₁₂ haloalkyl, C₁-C₁₂ alkoxy, C₂-C₁₈ (hetero)aryloxy, C₂-C₁₈ (hetero)arylamino, C₂-C₁₂ carboxylic acid, C₂-C₁₂ carboxylate ester, and C₁-C₁₂ alkoxy;
each Z is independently CH₂, O, or NR₄;
each f is independently an integer from 0 to 50; and
each n is independently an integer from 1 to 20.

A fluorescent polymer dye may be any dye disclosed in US2019/0144601, which is incorporated herein by reference in its entirety. The fluorescent polymer dye may be, for example, a violet fluorescent polymer dye having the structure of Formula VI:

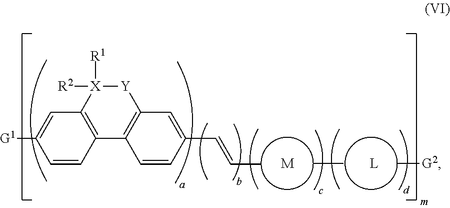

wherein
each X is independently selected from the group consisting of a C and Si;
each Y is independently selected from the group consisting of a bond, CR¹R² and SiR¹R²;
when Y is a bond X is directly bonded to both rings;
each R¹ is independently selected from the group consisting of polyethylene glycol (PEG), ammonium alkyl salt, ammonium alkyloxy salt, ammonium oligoether salt, sulfonate alkyl salt, sulfonate alkoxy salt, sulfonate oligoether salt, sulfonamido oligoether, and

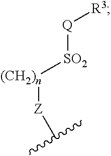

each R² is independently selected from the group consisting of H, alkyl, alkene, alkyne, cycloalkyl, haloalkyl, alkoxy, (hetero)aryloxy, aryl, (hetero)arylamino, PEG, ammonium alkyl salt, ammonium alkyloxy salt, ammonium oligoether salt, sulfonate alkyl salt, sulfonate alkoxy salt, sulfonate oligoether salt, sulfonamido oligoether, and

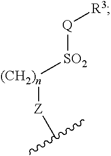

each R³ is independently selected from the group consisting of H, alkyl, alkene, alkyne, cycloalkyl, haloalkyl, alkoxy, (hetero)aryloxy, aryl, (hetero)arylamino, and PEG; each R⁴ is independently selected from the group consisting of H, alkyl, PEG, a water-solubilizing moiety, a linker moiety, a chromophore, carboxylic amine, amine, carbamate, carboxylic acid, carboxylate ester, maleimide, activated ester, N-hydroxysuccinimidyl, hydrazine, hydrazide, hydrazone, azide, alkyne, aldehyde, or thiol, or protected groups thereof;
each Z is independently CH₂, O, or NR⁴;
each Q is independently selected from the group consisting of a bond, NH, NR⁴, and CH₂;
each M is independently an electron rich linker unit capable of altering the polymer band gap and is evenly or randomly distributed along the polymer main chain;
each R⁴ is a non-ionic side group capable of imparting solubility in water in excess if 10 mg/mL and is each independently selected from the group consisting of halogen, hydroxyl, C₁-C₁₂ alkyl, C₂-C₁₂ alkene, C₂-C₁₂ alkyne, C₁-C₁₂ cycloalkyl, C₁-C₁₂ haloalkyl, C₁-C₁₂ alkoxy, C₂-C₁₈ (hetero)aryloxy, C₂-C₁₈ (hetero)arylamino, (CH₂)_{x′}(OCH₂—CH₂)_{y}·OCH₃ where each x′ is independently an integer from 0-20; each y′ is independently an integer from 0-50, and a C₂-C₁₈ (hetero)aryl group;
each optional linker L is an aryl or heteroaryl group evenly or randomly distributed along the polymer main chain and may be substituted with one or more pendant chains terminated with a functional group selected from the group consisting of amine, carbamate, carboxylic acid, carboxylate, maleimide, activated ester, N-hydroxysuccinimidyl, hydrazine, hydrazide, hydrazone, azide, alkyne, aldehyde, thiol, and protected groups thereof for conjugation to another substrate, acceptor dye, molecule or binding agent;
each G₁ and G₂ are each independently selected from the group consisting of hydrogen, halogen, alkyne, optionally substituted aryl, optionally substituted heteroaryl, halogen substituted aryl, silyl, diazonium salt, triflate, acetyloxy, azide, sulfonate, phosphate, boronic acid substituted aryl, boronic ester substituted aryl, boronic ester, boronic acid, optionally substituted dihydrophenanthrene (DHP), optionally substituted fluorene, aryl or heteroaryl substituted with one or more pendant chains terminated with a functional group selected from amine, carbamate, carboxylic acid, carboxylate, maleimide, activated ester, N-hydroxysuccinimidyl, hydrazine, hydrazide, hydrazone, azide, alkyne, aldehyde, thiol, and protected groups thereof for conjugation to a substrate or a binding agent;
a, c, and d, define the mol % of each unit within the structure which each can be evenly or randomly repeated and where a is a mol % from 10 to 100%, c is a mol % from 0 to 90%, and each d is a mol % from 0 to 25%;
each b is independently 0 or 1;
m is an integer from 1 to about 10,000; and
each n is independently an integer from 1 to 20.

In some cases, each M may independently be selected from the group consisting of

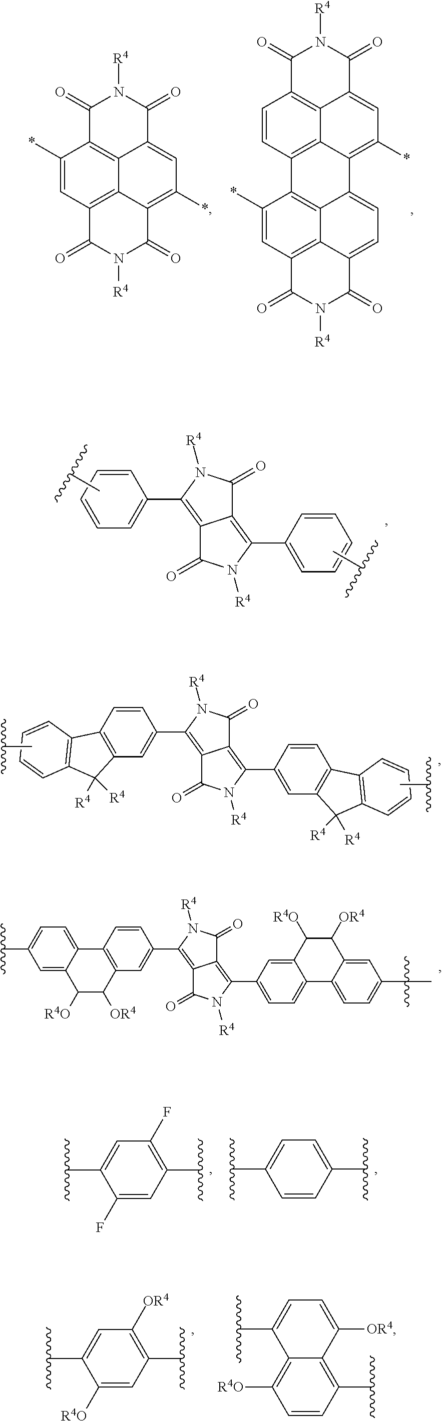

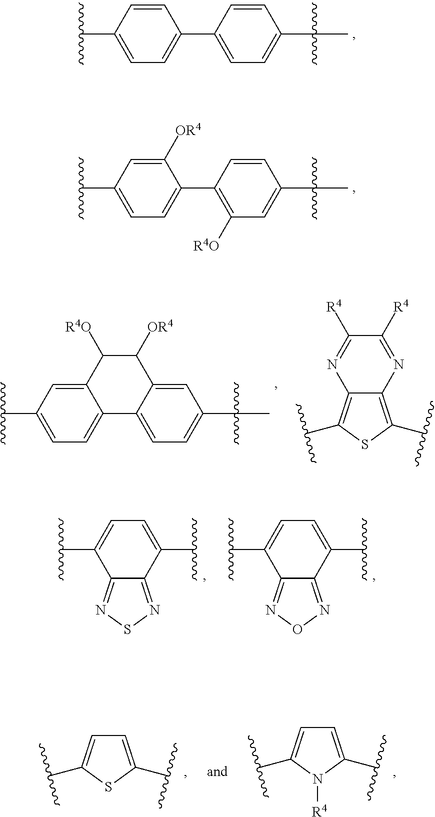

wherein each R′ is a non-ionic side group capable of imparting solubility in water in excess if 10 mg/mL and is each independently selected from the group consisting of halogen, hydroxyl, C₁-C₁₂ alkyl, C₂-C₁₂ alkene, C₂-C₁₂ alkyne, C₃-C₁₂ cycloalkyl, C₁-C₁₂ haloalkyl, C₁-C₁₂ alkoxy, C₂-C₁₈ (hetero)aryloxy, C₂-C₁₈ (hetero)arylamino, (CH₂)_{x′}(OCH₂—CH₂)_{y′}OCH₃ where each x′ is independently an integer from 0-20; each y′ is independently an integer from 0-50, and a C₂-C₁₈ (hetero)aryl group.

In some cases, the fluorescent polymer dye may have the structure of Formula VII:

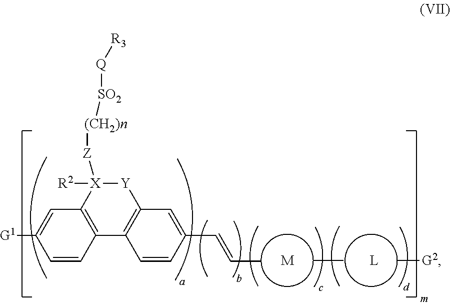

wherein X, Y, R², R³, G¹, G², L, M, Q, Z, a, b, c, d, m and n are as defined previously.

In some cases, the fluorescent polymer dye may have the structure of Formula VIII:

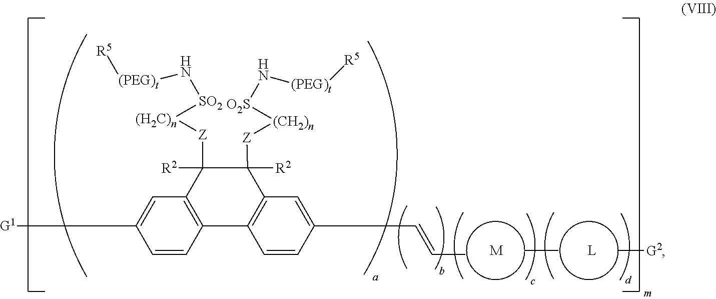

wherein
X, Y, R², R⁴, R⁵, G¹, G², L, M, Q, Z, a, b, c, d, m and n are as defined previously;
each f is independently an integer from 0 to 50; and
and each R⁵ is independently selected from the group consisting of H, C₁-C₁₂ alkyl, C₂-C₁₂ alkene, C₂-C₁₂ alkyne, C₃-C₁₂ cycloalkyl, C₁-C₁₂ haloalkyl, C₁-C₁₂ alkoxy, C₂-C₁₈ (hetero)aryloxy, C₂-C₁₈ (hetero)arylamino, and C₁-C₁₂ alkoxy.

In some cases, the fluorescent polymer dye may have the structure of Formula IX:

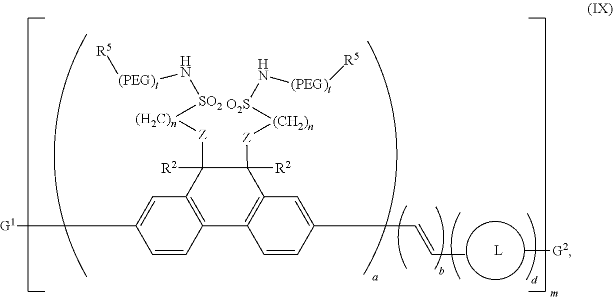

wherein R², R⁴, R⁵ G₁, G₂, L, Z, a, b, d, f, m and n are as defined previously.

In some cases, the fluorescent polymer dye may have the structure of Formula X:

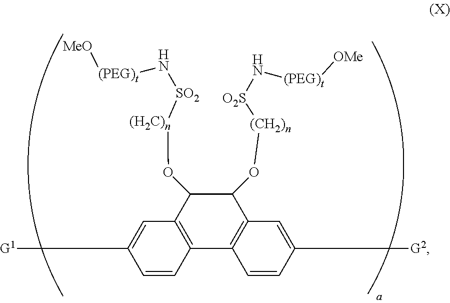

wherein G¹, G², a, f, and n are as defined previously.

In some cases, the fluorescent polymer dye may be a copolymer having the structure of Formula XI:

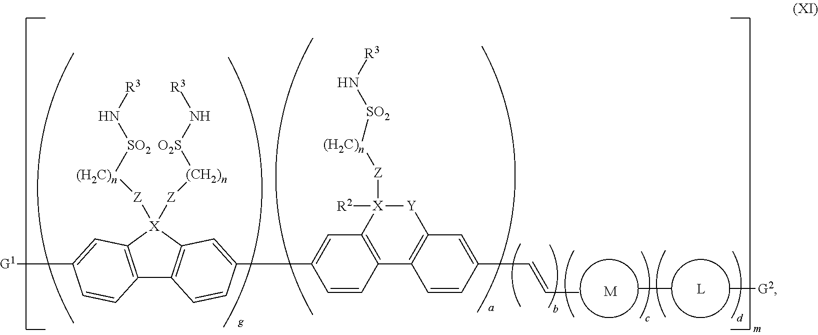

wherein X, Y, R¹, R², R³, R⁴, G¹, G², L, M, Q, Z, a, b, c, d, m and n are as defined previously; and
g and a together is a mol % from 10 to 100%.

In some cases, the fluorescent polymer dye may be a copolymer having the structure of Formula XII:

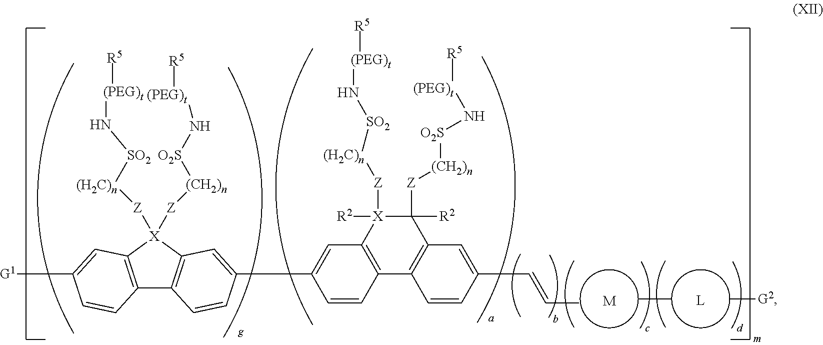

wherein
X, Y, R², R⁴, R⁵, G¹, G², L, M, Q, Z, a, b, c, d, m and n are as defined previously;
each f is independently an integer from 0 to 50; and
each g and a together is a mol % from 10 to 100%.

In some cases, the fluorescent polymer dye may be a copolymer having the structure of Formula XIII:

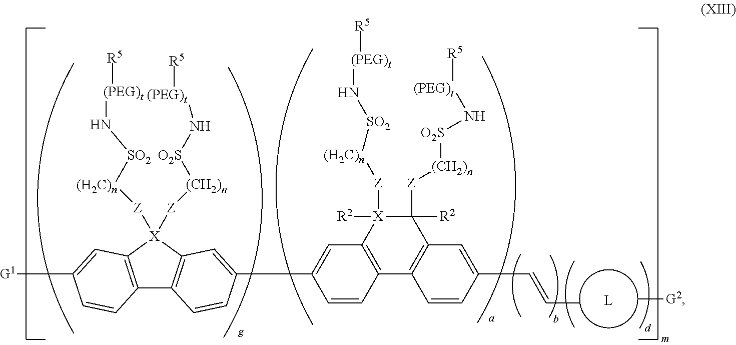

wherein
X, R², R⁴, R⁵, G¹, G², L, Z, a, b, d, f, m and n are as defined previously; and
each g and a together is a mol % from 10 to 100%.

In some cases, the fluorescent polymer dye may be a copolymer having the structure of Formula XIV:

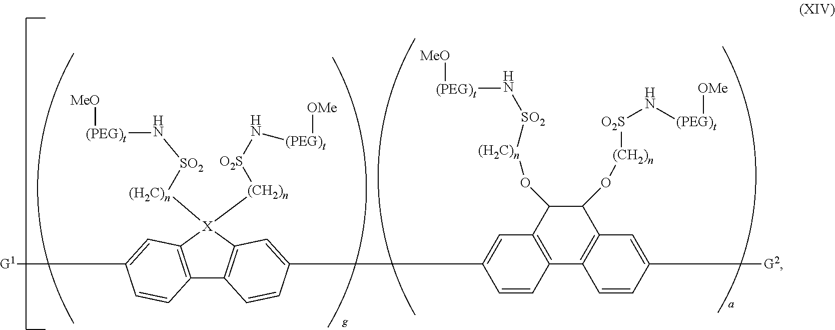

wherein
G¹, G², a, f, and n are as defined previously; and
each g and a together is a mol % from 10 to 100%.

The fluorescent polymer dye may be prepared by polymerization of water-soluble monomers such as a monomer A and a monomer B, which leads to formation of a highly conjugated fluorescent backbone. Capping may be carried out on the polymer by activation using appropriate functionalities, which results in a polymer capable of being conjugated to a binding partner. Alternatively, a monomer A or a monomer B may be directly modified by activation using appropriate functionalities, for example, according to US 2020/0190253, which is incorporated by reference herein in its entirety. The activated polymers may be conjugated to a binding partner. Any appropriate binding partner may be employed, for example, an antibody, followed by purification, for example, by using standard procedures.

Polymer dyes are commercially available. For example, SuperNova™ (“SN”) v428 (Beckman Coulter, Inc.) is a polymer dye optimally excited by the violet laser (405 nm) with an excitation maximum of 414 nm, an emission peak of 428 nm, and can be detected using a 450/50 bandpass filter or equivalent. SN v428 is a bright polymer dye that can be activated with amine for tandem dyes, followed by activation for tandem conjugates. The rigidity of the polymer dye structure may help reduce rotational energy leading to brighter emissions. This may help achieve optimized FRET (fluorescence resonance energy transfer) efficiency and increased stability.

SN v428 is one of the brightest dyes excitable by the violet laser, so it is particularly suited for assessing dimly expressed markers. SN conjugated antibodies may include anti-CD19 antibody-SN v428, anti-CD22 antibody-SN v428, anti-CD25 antibody-SN v428, and anti-CD38 antibody-SN v428 antibody-polymeric dye conjugates. SN v605 and SN v786 (Beckman Coulter, Inc.) are tandem polymer dyes, derived from the core SN v428 polymer dye. Both share the same absorbance characteristics, with maximum excitation at 414 nm. With emission peaks for SN v605 and SN v786 at 605 nm and 786 nm respectively, they are optimally detected using the 610/2 and 780/60 nm bandpass filters of the flow cytometer.

The fluorescent polymer dye may be a fluorescent polymeric dye commercially available from Becton Dickinson, including Brilliant™ Blue, Brilliant™ Violet and Brilliant™ Ultra Violet dyes. The fluorescent polymer dye may be a fluorescent polymeric dye commercially available from ThermoFisher Scientific, including Super Bright 436, Super Bright 600, Super Bright 645, Super Bright 702, and Super Bright 780 dyes.

### Water-Soluble Monomer

The “water-soluble monomer” may be a monomeric unit comprising an aryl moiety or heteroaryl moiety, each optionally having one or more water-solubilizing moieties attached thereto. The water-soluble moiety may be one or more PEG moieties. The water-soluble monomer may be appropriate for use in preparation of at least one of the plurality of the fluorescent polymer dyes having a monomer A subunit, a monomer B subunit, or a combination of monomer A and monomer B subunits. The water-soluble monomer may be a dihydrophenanthrene (DHP)-based water-soluble monomer. The water-soluble monomer may be a fluorene-based water-soluble monomer.

The water-soluble monomer may be a dihydrophenanthrene (DHP)-based monomer having a chemical structure according to Formula (I):

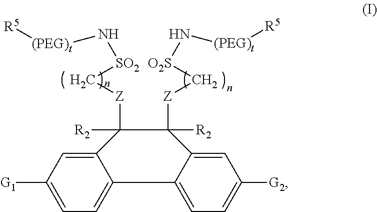

wherein
each G¹, G² is independently selected from the group consisting of halogen, alkyl, PEG, hydrogen, alkyne, optionally substituted aryl, optionally substituted heteroaryl, halogen substituted aryl, silyl, diazonium salt, triflate, acetyloxy, azide, sulfonate, phosphate, optionally substituted tetrahydropyrene (THP), optionally substituted fluorene, optionally substituted dihydrophenanthrene (DHP), aryl or heteroaryl substituted with one or more pendant chains terminated with a functional group selected from amine, carbamate, carboxylic acid, carboxylate, maleimide, activated ester, N-hydroxysuccinimidyl, hydrazine, hydrazide, hydrazone, azide, alkyne, aldehyde, and thiol;
each R² is independently selected from the group consisting of a water-solubilizing moiety, alkene, alkyne, cycloalkyl, haloalkyl, (hetero)aryloxy, (hetero)arylamino, sulfonamide-PEG, phosphoramide-PEG, ammonium alkyl salt, ammonium alkyloxy salt, ammonium oligoether salt, sulfonate alkyl salt, sulfonate alkoxy salt, sulfonate oligoether salt, sulfonamido oligoether, sulfonamide, sulfinamide, phosphonamidate, phosphinamide,

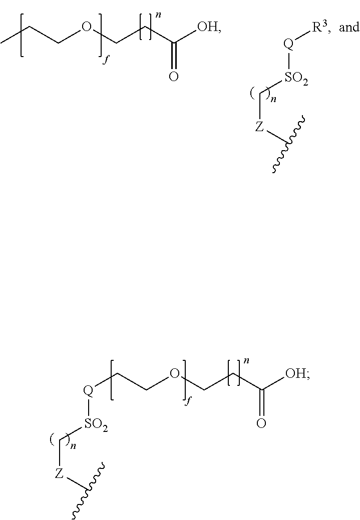

each R³ is a water-solubilizing moiety;
each R⁴ is independently selected from the group consisting of H, alkyl, PEG, a water-solubilizing moiety, a linker moiety, a chromophore, carboxylic amine, amine, carbamate, carboxylic acid, carboxylate ester, maleimide, activated ester, N-hydroxysuccinimidyl, hydrazine, hydrazide, hydrazone, azide, alkyne, aldehyde, or thiol, or protected groups thereof;
each R⁵ is independently selected from the group consisting of H, hydroxyl, C₁-C₁₂ alkyl, C₂-C₁₂ alkene, C₂-C₁₂ alkyne, C₃-C₁₂ cycloalkyl, C₁-C₁₂ haloalkyl, C₁-C₁₂ alkoxy, C₂-C₁₈ (hetero)aryloxy, C₂-C₁₈ (hetero)arylamino, C₂-C₁₂ carboxylic acid, C₂-C₁₂ carboxylate ester, and C₁-C₁₂ alkoxy;
each Q is independently a bond, NR⁴, or —CH₂;
each Z is independently CH₂, O, or NR⁴;
each f is independently an integer from 0 to 50; and
each n is independently an integer from 1 to 20.

The water-soluble monomer may be a fluorene-based monomer having a chemical structure according to Formula (II):

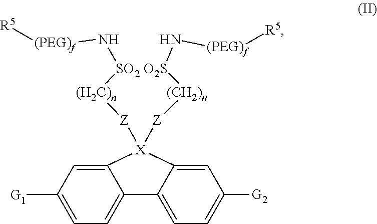

wherein
each G¹, G² is independently selected from the group consisting of halogen, alkyl, PEG, hydrogen, alkyne, optionally substituted aryl, optionally substituted heteroaryl, halogen substituted aryl, silyl, diazonium salt, triflate, acetyloxy, azide, sulfonate, phosphate, optionally substituted tetrahydropyrene (THP), optionally substituted fluorene, optionally substituted dihydrophenanthrene (DHP), aryl or heteroaryl substituted with one or more pendant chains terminated with a functional group selected from amine, carbamate, carboxylic acid, carboxylate, maleimide, activated ester, N-hydroxysuccinimidyl, hydrazine, hydrazide, hydrazone, azide, alkyne, aldehyde, and thiol;
each X is C or Si;
each R⁴ is independently selected from the group consisting of H, alkyl, PEG, a water-solubilizing moiety, a linker moiety, a chromophore, carboxylic amine, amine, carbamate, carboxylic acid, carboxylate ester, maleimide, activated ester, N-hydroxysuccinimidyl, hydrazine, hydrazide, hydrazone, azide, alkyne, aldehyde, or thiol, or protected groups thereof;
each R⁵ is independently selected from the group consisting of H, hydroxyl, C₁-C₁₂ alkyl, C₂-C₁₂ alkene, C₂-C₁₂ alkyne, C₃-C₁₂ cycloalkyl, C₁-C₁₂ haloalkyl, C₁-C₁₂ alkoxy, C₂-C₁₈ (hetero)aryloxy, C₂-C₁₈ (hetero)arylamino, C₂-C₁₂ carboxylic acid, C₂-C₁₂ carboxylate ester, and C₁-C₁₂ alkoxy;
each Z is independently CH₂, O, or NR⁴;
each f is independently an integer from 0 to 50; and
each n is independently an integer from 1 to 20.

The water-soluble monomer may be a dihydrophenanthrene (DHP)-based monomer having a chemical structure according to Formula (III):

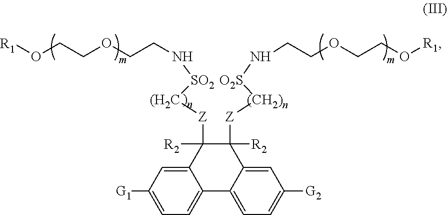

wherein each G₁, G₂ is independently halo (F, Cl, Br, I); each Z is independently selected from the group consisting of O, CH₂, and NH; each R₁ is independently alkyl (C₁-C₃); each R² is independently H, alkyl (C₁-C₆); each n is independently 1-6; and each m is independently 5-50. In some embodiments, G₁, G₂ are each Br; each Z is O; each R₁ is CH₃; each R₂ is H; each n is independently 2-4; and each m is independently 5-20. In some embodiments, each n is 3; and each m is independently 11 or 12.

### Labeled Binding Partner

Dyes can be conjugated to binding partners by various linking chemistry between reactive pairs located in the binding partners and the labels. The reactive pairs can include, but are not limited to, maleimide/thiol, succimidylester (NHS ester)/amine, azide chemistry, carboxy/EDC (1-Ethyl-3-[3-dimethylaminopropyl]carbodiimide Hydrochloride)/amine, amine/Sulfo-SMCC (Sulfosuccinimidyl 4-[N-maleimidom ethyl]cyclohexane-1-carboxylate)/thiol, and amine/BMPH (N-[^{˜}-Maleimidopropionic acid]hydrazide. TFA)/thiol.

Fluorescent polymer dyes feature termini on the conjugated polymer chains that can include a functional group that provides for conjugation. In some cases, such functionality is referred to as an end linker. With these end linkers, a covalent bond can be formed to attach a binding partner such as, for example, a protein, peptide, affinity ligand, antibody, antibody fragment, polynucleotide, or aptamer. Additionally, orthogonal functional groups can be installed along the conjugated polymer chain that can be used for either conjugation or the attachment of acceptor signaling chromophores in donor acceptor polymeric tandem dyes.

Methods for performing the conjugation are well known in the art. Commercial kits for performing the conjugation are also readily available, e.g., from Innova Biosciences (Cambridge, UK), Novus Biologicals (Littleton, Colo.), Thermo Fisher Scientific (Waltham, Mass.).

### Dry Down Process

Dried reagent technology may be used to increase stability of biomolecules. A drying process may be used to create a uniform reagent layer, for example, at the bottom of a tube. Dry reagents do not require refrigeration. Dry reagents may be stored at room temperature. Antibody panels may be supplied in a single-use cocktail. Antibody panels may be provided in a variety of substrates including tube or plate formats. Reagents may be used for drying conjugated antibodies, stabilizing them for room temperature storage. Reagent format may be tailored to combine different reagents, creating an antibody cocktail. An antibody cocktail may include a multiplicity of antibody-dye-conjugates, for example, one or more, two or more, three or more, four or more, five or more, six or more, seven or more, eight or more, nine or more, 10 or more, 11 or more, or 12 or more antibody-conjugates, or 1-20, 2-15, or 3-12 different antibody-dye conjugates.

The dried reagent technology may allow the ability to dry enumeration beads with the cocktail. The binding partner-dye conjugates, including antibody-dye and antibody-polymer dye conjugates may be used in flow cytometry assays. Any appropriate drying process may be employed that dries reagents creating a uniform layer at the bottom of a tube or well.

The prior art “Reagent Buffer” formulation (RB) comprising sacrificial protein, a carbohydrate stabilizer, antimicrobial agent and buffering agent was previously developed for drying different monomeric conjugate dyes in a single tube without altering functionality (affinity to antigens) and physical properties (such as brightness or fluorescence). The prior art RB formulation does not include a zwitterionic surfactant or a water-soluble monomer. Physical properties refers to brightness of the conjugate and its spillover into other channels.

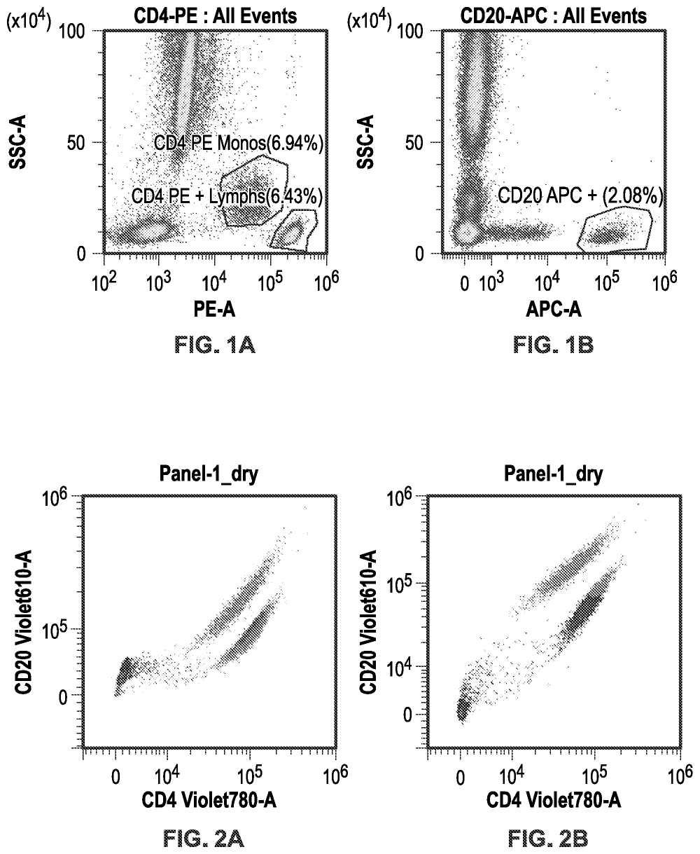

As illustrated in FIG. 1A, desirable flow cytometry results using monomeric CD4-PE dye conjugate are exhibited when dried using prior dry down technology and reconstituted with a blood sample; including desirable functionality, physical properties, and resolution of CD4 PE monocytes and CD4 PE+ lymphocytes cell populations. Similarly, as illustrated in FIG. 1B, desirable flow cytometry results of monomeric CD20 APC dye conjugate are exhibited when dried using prior art technology and reconstituted with a blood sample, including desirable functionality, physical properties, and resolution of CD20 APC+ cell population.

Prior drying technology such as RB, when used to dry down two or more polymer dye conjugates in a tube, was found to alter the integrity of the polymer structure, resulting in aggregation of polymer dye conjugates. Aggregation increases non-specific interaction and creates staining artifacts. FIGS. 2A and 2B depict the inability of prior technology to resolve cell population stained using polymer dye due to aggregation of polymer dye conjugates when two polymer dye conjugates were dried using prior drying technology.

FIG. 2A shows undesirable flow cytometry results of CD20-SNv605 and CD4-SNv786 polymer dye antibody conjugates when mixed and dried using prior drying technology without compensation. Non-specific interaction and an inability to resolve populations on x and y axis was exhibited.

FIG. 2B shows undesirable flow cytometry results of CD20-SNv605 and CD4-SNv786 polymer dye antibody conjugates when mixed and dried using prior drying technology with compensation. The inability to prevent non-specific interaction was deemed to be due to aggregation of polymer dye antibody conjugates when the two polymer dyes were dried using prior drying technology.

Therefore, a need exists for a new buffer composition in order to keep fluorescent dye conjugates stable and decrease aggregation while drying.

In order to overcome the technical problem stated above, initially the fluorescent dye conjugate should be stable in its liquid state when mixed. A mixture of two fluorescent dye conjugates, even in liquid state, when mixed together was found to result in increased non-specific interaction, but spillover could be compensated. To overcome the limitation in liquid state, commercial buffers were tested including from BD Biosciences (Brilliant Stain Buffer, Catalog No: 563794) and Thermo Fisher (Super Bright Complete Staining Buffer, catalog number: SB-4401-42). Use of Brilliant Stain Buffer or Super Bright Complete Staining Buffer during drying of polymer dye conjugates did not resolve the problems of poor stability and aggregation.

Other techniques were adopted to dry the fluorescent dye conjugates that involved membrane immobilization of fluorescent dye conjugates onto substrate separately as described in US 2019/0242882, which is incorporated herein by reference. From these attempts, it was concluded that fluorescent dye conjugates could be dried in different spots separately onto substrate membrane, but two fluorescent dye conjugates could not be mixed and dried. Disadvantages of drying polymer dye conjugates separately include the fact that drying fluorescent dye conjugates onto cellulose membrane involves deviation from current drying techniques, inconvenience in maintaining cellulose membranes inside DURAClone™ tube substrates, and acceptance of spillover in other channels with high compensation values. These experiments indicated that more than one fluorescent dye conjugate could be dried only in the presence of buffer that forms a barrier and preserves the individuality of each fluorescent dye conjugate during the drying process.

Fluorescent dye conjugates may be employed in multi-color dry reagents (for example, DURAClone™ Tubes, Beckman Coulter, Inc.). A multi-color dry reagent is a cocktail of different fluorescent dye conjugates (CD4-FITC, CD8-PE, CD20-APC, CD3-PC5.5, etc.) that may be used directly to stain blood and analyze it in a flow cytometer. Compared to the existing monomeric conjugate dyes, polymer dye conjugates are different in its structure and complexity.

To achieve a multi-color dry reagent cocktail with different conjugates, prior drying technology is employed. Conventional dyes such as FITC, PE, ECD, PC5, PC5.5, PC7, APC, AA700, AA750, PBE and KrO can be dried using prior drying technology.

With the introduction of polymer dye conjugates, prior drying technology was found to be ineffective while drying multiple polymer dye antibody conjugates in a cocktail. These polymer dye conjugates tend to non-specifically interact and lead to the difficulties in resolving the population, which might lead to challenges in identification of the desired cell populations in a given sample.

### Compositions

In order to overcome the limitations of prior drying technology, a novel “Dry Mix” (“DM”) buffer has been developed that may be used to dry one or more fluorescent (polymer or monomer) dye conjugates in a dye cocktail. The dye cocktail may comprise more than one fluorescent polymer dye conjugate. The dye cocktail may comprise more than one conventional non-polymer fluorescent dye conjugate. The dye cocktail may comprise a combination of one or more fluorescent polymer dye conjugate(s) and one or more conventional non-polymer fluorescent dye conjugates.

The fluorescent dye conjugates may be dried along with other conventional dyes in a cocktail. Several components were evaluated for use in the DM buffer formulation according to the protocol of example 1. The experimental outcomes for those components evaluated during the development of DM buffer are shown in Table 1.

The inventive DM buffer formulation will typically be an aqueous solution comprising a water-soluble monomer, a protein stabilizer, a carbohydrate stabilizer, a zwitterionic surfactant, and optionally a colorant and optionally a preservative.

Stabilizers used in the solutions may include a protein stabilizer (e.g., bovine serum albumin, gelatin, casein), and a carbohydrate stabilizer (e.g., trehalose, dextrose, sucrose). In some embodiments, the stabilizers can facilitate the attachment of the dry component to the substrate so that it remains at the bottom of the tube and will not be blown away or stick to the cap when the reaction vessel is opened. The DM buffer composition may comprise per test: 200 to 800 μg of the water-soluble monomer; 2000 to 3000 μg of the carbohydrate stabilizer; 8.4 to 72 μg of the protein stabilizer; and 2 to 15 μg of the zwitterionic surfactant.

### Diluents

Diluents for the DM buffer may be selected from the group consisting of water and an isotonic buffer. The water may be a deionized water (DI water). The isotonic buffer may be a PBS (phosphate buffer saline) buffer.

### Protein Stabilizers

The term “protein stabilizer” refers to a protein that serves to reduce non-specific binding, for example, to reduce cell-cell interactions, or to help prevent non-specific binding between an antibody and a non-target molecule. Protein stabilizers may include bovine serum albumin (BSA), various gelatins, and casein. Various protein stabilizers were evaluated in DM buffer compositions, as shown in Table 1. The protein stabilizer may be a casein. The protein stabilizer may be a gelatin. In some embodiments, the protein stabilizer may be BSA. In some embodiments, the protein stabilizer is not BSA. The dry down buffer may include one or more protein stabilizers.

Gelatin or gelatine is a protein, commonly derived from collagen taken from animal body parts. It is brittle when dry and gummy when moist. It may also be referred to as hydrolyzed collagen, collagen hydrolysate, gelatine hydrolysate, hydrolyzed gelatine, and collagen peptides after it has undergone hydrolysis. Several types of gelatin are commercially available including gelatin-type A, gelatin-type B, Prionex® highly purified gelatin Type A, and gelatin-cold water fish. Each of these was evaluated as candidate DM buffer components. As reported in Table 1, Gelatin-Type A in dry format exhibited more spread of the negative population compared to corresponding liquid cocktails. Gelatin Type B dry mix was able to prevent the non-specific interactions but the preparation of stocks was difficult. The effective concentration of Gelatin Type B was in a range of 150 micrograms to 450 micrograms per test. Prionex® highly purified Type A gelatin at a concentration equivalent to Gelatin-Type B was efficient in preventing non-specific interaction but the stability was somewhat reduced. The concentration of ready-made solution of Prionex® Highly purified Type A gelatin was quantified and effective dry mix concentration was in a range of 67 micrograms to 135 micrograms per test. Gelatin-cold water fish was equivalent to Gelatin-Type B in terms of performance. Effective concentration of Gelatin-cold water fish was in a range of 150 micrograms to 450 micrograms per test.

Casein is a family of phosphoproteins (alphaS1, alphaS2, beta, and kappa). These proteins are found in mammalian milk, comprising about 80% of the proteins found in cow's milk. One common form is sodium caseinate. Casein contains a high number of proline amino acid residues, which hinder formation of common secondary structural motifs of proteins. Casein does not contain disulfide bridges, so has relatively little tertiary structure. Casein 10× blocking buffer (in a range of from about 14 mg/mL to about 18 mg/mL) was employed as a one of dry mix components. Casein at 5× (2 time diluted) and 2.5× (4 time diluted) concentrations could effectively prevent interaction of two polymer dye antibody conjugates with addition of one more polymer conjugate.

### Carbohydrate Stabilizers

A “carbohydrate stabilizer” is a carbohydrate molecule used to help increase stability of dye antibody conjugates in solution, upon drying to substrate, and/or upon reconstitution with biological sample.

Candidate polysaccharides were evaluated. Carrageenan is a sulfated anionic polysaccharide. Carrageenan preparation of stock was found to be challenging. Addition of Carrageenan to DM buffer composition increased non-specific binding in granulocytes. Sodium alginate is a sodium salt of alginic acid which is a linear polysaccharide comprising homopolymeric blocks of (1→4)-linked beta-D-mannuronate and alpha-L-guluronate residues. Preparation of stock was difficult and overall spread of granulocytes, monocytes, and lymphocytes was higher when sodium alginate was added to DM buffer composition.

The carbohydrate stabilizer may be a disaccharide. The disaccharide may be a trehalose, a sucrose, a maltose, a cellobiose, a melibiose, or a hydrate, or a salt thereof. In some embodiments, the disaccharide is a trehalose or a hydrate thereof. Trehalose is a non-reducing disaccharide having a 1,1-glycosidic bond between two alpha-glucose units. The trehalose may be trehalose dihydrate. The disaccharide stabilizer may be present in a range of from about 2000 to about 3000 micrograms per test, or about 2200 micrograms to about 2800 micrograms per test, or about 2500 micrograms per test, or in aqueous buffer solution at about 400 mg/mL in stock preparation.

Trehalose derivatives were also evaluated including trehalose decanoate, trehalose tetradecanoate, and trehalose hexadecanoate. For all three at a dose equivalent to trehalose dihydrate, the compounds lyse all cells completely, and lowering the dose will not dry the tubes.

### Surfactants

Various types of surfactants were explored for preventing non-specific interactions in the DM buffer formulations. (See Table 1).

Anionic surfactants were evaluated including alkyl sulfates and alkylsulfonates having an alkyl group of at least ten carbons. N-lauryl sarcosine sodium salt, also known as sarkosyl, CH₃(CH₂)₁₀CO—N(CH₃)—CH₂COONa, is an anionic surfactant. N-lauryl sarcosine sodium salt was found to prevent the non-specific binding on monocytes and granulocytes, but was not able to prevent polymer-polymer interactions. Lignosulfonic acid (“LSA”), 3-(2-hydroxy-3-methoxyphenyl)-2-[2-methoxy-4-(3-sulfopropyl) phenoxy]propane-1-sulfonic acid, is an anionic surfactant. Negative background of SN v605 and SN v786 population increased compared to DM buffer when LSA was added.

Nonionic surfactants were evaluated for possible use in DM buffer formulations. Polysorbate 80 was tested as a nonionic surfactant. The term “ester-linked nonionic surfactant” refers to a nonionic organic compound containing hydrophobic and hydrophilic groups connected by or comprising an ester linkage. Examples of ester-linked nonionic surfactants include polyoxyethylene glycol sorbitan esters (Polysorbates, TWEENs), sorbitan alkyl esters (Spans). Nonspecific monocyte pull-out and spread of the population with SN v605 conjugate was contained at 0.015% and 0.075% polysorbate 80 in dry mix.

Zwitterionic surfactants were evaluated for possible use in DM buffer formulations. Zwitterionic surfactants Empigen® BB (Huntsman Corporation), also known as N,N-dimethyl-N-dodecylglycine, or N-(alkylC₁₀-C₁₆)—N,N-dimethylglycine betaine; 3-(N,N-dimethylmyristylammonio propane sulfonate (DMMA), also known as 3-(N,N-dimethyltetradecylammonio) propanesulfonate, myristyl sulfobetaine, CH₃(CH₂)₁₃N⁺(CH₃)₂CH₂CH₂CH₂SO₃⁻); and 3-[N,N-dimethyl (3-palmitoylaminopropyl) ammonio]-propane sulfonate (DMPA), also known as Zwittergent® 3-16 detergent (Merck KGaA, Darmstadt, Germany) were evaluated in DM buffer compositions. Empigen® BB could prevent the non-specific binding on monocytes and granulocytes but was not able to prevent polymer interactions. DMPA precipitates at room temperature but was functionally equivalent to Empigen® BB. The effective concentration range of DMPA was 0.002% to 0.006%. DMMA was functionally equivalent to Empigen® BB, therefore was considered for further testing. The effective concentration of DMMA was found to be in a range of 0.002% to 0.037%, or 0.004% to 0.018%.

In some embodiments, the zwitterionic surfactant has a chemical structure according to Formula (XV):

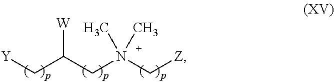

wherein Y═CO₂—, or SO₃—, W═H or OH, Z═CH₃ or NHC(O)R, where R═C₁₋₁₅ alkyl; independently each p=0 or 1; and q=0-21. In some embodiments, W═H, Z═CH₃, and q=11-15. In some embodiments, the zwitterionic surfactant may be DMMA, DMPA, N-(alkylC₁₀-C₁₆)—N,N-dimethylglycine betaine, lauryl hydroxysultaine, lauryl sultaine, myristyl betaine, cetyl betaine, decyl betaine, lauryl betaine, behenyl betaine, cocamidopropyl betaine, or cocamidopropyl hydroxy sultaine. In some embodiments, the zwitterionic surfactant may be DMMA, DMPA, N-(alkylC₁₀-C₁₆)—N,N-dimethylglycine betaine.

### Antioxidant

Antioxidant compounds were evaluated as candidate components of the DM buffer formulations. The antioxidant may comprise one or more, two or more, or three or more carboxylic acid or carboxylate moieties and a C₁-C₈, or C₂-C₆ aliphatic moiety. The aliphatic moiety may be a straight-chain, branched-chain, or cyclo-alkyl, or alkenyl moiety. For example, L-ascorbic acid and citric acid were evaluated as antioxidants. Non-specific monocyte pull out with SN v605 conjugate were less at 0.2 mM and 0.6 mM concentrations of L-ascorbic acid. 0.6 mM L-ascorbic acid had less spread of SN v786 positive population in V610 channel. Citric acid reduced the degranulation of granulocytes and non-specific monocyte pull out in SN v605 conjugate was less at all concentrations. However, citric acid did not have additional impact in containing the spread of the population.

### Monomers

In order to prevent non-specific interactions between fluorescent dye conjugates, and in particular between fluorescent polymer dye conjugates, various water-soluble monomer species were investigated. The monomers employed were selected from synthetic monomers used in preparation of conjugate polymer dyes. A polymer dye may be a water-soluble conjugated polymer, including fluorescent polymers having monomer A subunits and monomer B subunits. For example, the monomer A or monomer B may comprise a DHP. Polymer dyes and monomers are described in US2020/0190253, which is incorporated herein by reference in its entirety. In some embodiments, a conjugated polymer dye containing a DHP backbone may be employed. In some embodiments, monomer A, having a 9,10-dihydrophenanthrene DHP-based structures were investigated for use in DM buffer composition formulation and tested according to example 1.

The water-soluble monomer may be a monomeric unit comprising an aryl moiety or heteroaryl moiety, each optionally having a water-soluble moiety attached thereto. The water-soluble moiety may be one or more PEG moieties. The water-soluble monomer may be appropriate for use in preparation of at least one of the plurality of the fluorescent polymer dyes having a monomer A subunit, a monomer B subunit, or a combination of monomer A and monomer B subunits. The water-soluble monomer may be a DHP-based water-soluble monomer. The water-soluble monomer may be a fluorene-based water-soluble monomer.

The water-soluble monomer may be a DHP-based monomer having a chemical structure according to Formula (I):

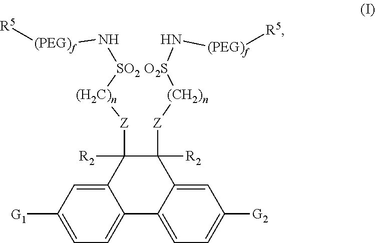

wherein
each G₁, G₂ is independently selected from the group consisting of halogen, alkyl, PEG, hydrogen, alkyne, optionally substituted aryl, optionally substituted heteroaryl, halogen substituted aryl, silyl, diazonium salt, triflate, acetyloxy, azide, sulfonate, phosphate, optionally substituted tetrahydropyrene (THP), optionally substituted fluorene, optionally substituted dihydrophenanthrene (DHP), aryl or heteroaryl substituted with one or more pendant chains terminated with a functional group selected from amine, carbamate, carboxylic acid, carboxylate, maleimide, activated ester, N-hydroxysuccinimidyl, hydrazine, hydrazide, hydrazone, azide, alkyne, aldehyde, and thiol;
each R² is independently selected from the group consisting of a water-solubilizing moiety, alkene, alkyne, cycloalkyl, haloalkyl, (hetero)aryloxy, (hetero)arylamino, sulfonamide-PEG, phosphoramide-PEG, ammonium alkyl salt, ammonium alkyloxy salt, ammonium oligoether salt, sulfonate alkyl salt, sulfonate alkoxy salt, sulfonate oligoether salt, sulfonamido oligoether, sulfonamide, sulfinamide, phosphonamidate, phosphinamide,

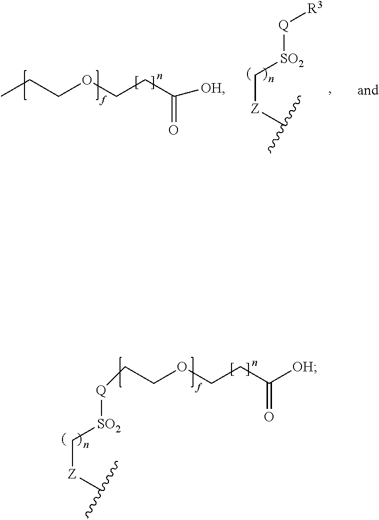

each R³ is a water-solubilizing moiety;
each R⁴ is independently selected from the group consisting of H, alkyl, PEG, a water-solubilizing moiety, a linker moiety, a chromophore, carboxylic amine, amine, carbamate, carboxylic acid, carboxylate ester, maleimide, activated ester, N-hydroxysuccinimidyl, hydrazine, hydrazide, hydrazone, azide, alkyne, aldehyde, or thiol, or protected groups thereof;
each R⁵ is independently selected from the group consisting of H, hydroxyl, C₁-C₁₂ alkyl, C₂-C₁₂ alkene, C₂-C₁₂ alkyne, C₃-C₁₂ cycloalkyl, C₁-C₁₂ haloalkyl, C₁-C₁₂ alkoxy, C₂-C₁₈ (hetero)aryloxy, C₂-C₁₈ (hetero)arylamino, C₂-C₁₂ carboxylic acid, C₂-C₁₂ carboxylate ester, and C₁-C₁₂ alkoxy;
each Q is independently a bond, NR₄, or —CH₂;
each Z is independently CH₂, O, or NR₄;
each f is independently an integer from 0 to 50; and
each n is independently an integer from 1 to 20.

In some embodiments, each G₁, G₂ is independently selected from the group consisting of halo (F, Cl, Br, I), C₁-C₆ alkyl, and PEG.

The water-soluble monomer may be a fluorene-based monomer having a chemical structure according to Formula (II):

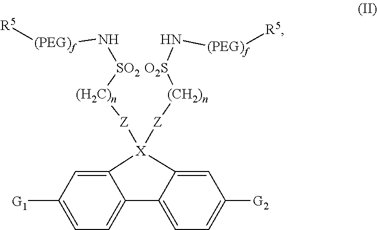

wherein
each G₁, G₂ is independently selected from the group consisting of halogen, alkyl, PEG, hydrogen, alkyne, optionally substituted aryl, optionally substituted heteroaryl, halogen substituted aryl, silyl, diazonium salt, triflate, acetyloxy, azide, sulfonate, phosphate, optionally substituted tetrahydropyrene (THP), optionally substituted fluorene, optionally substituted dihydrophenanthrene (DHP), aryl or heteroaryl substituted with one or more pendant chains terminated with a functional group selected from amine, carbamate, carboxylic acid, carboxylate, maleimide, activated ester, N-hydroxysuccinimidyl, hydrazine, hydrazide, hydrazone, azide, alkyne, aldehyde, and thiol;
each X is C or Si;
each R⁴ is independently selected from the group consisting of H, alkyl, PEG, a water-solubilizing moiety, a linker moiety, a chromophore, carboxylic amine, amine, carbamate, carboxylic acid, carboxylate ester, maleimide, activated ester, N-hydroxysuccinimidyl, hydrazine, hydrazide, hydrazone, azide, alkyne, aldehyde, or thiol, or protected groups thereof;
each R⁵ is independently selected from the group consisting of H, hydroxyl, C₁-C₁₂ alkyl, C₂-C₁₂ alkene, C₂-C₁₂ alkyne, C₃-C₁₂ cycloalkyl, C₁-C₁₂ haloalkyl, C₁—C₁₂ alkoxy, C₂-C₁₈ (hetero)aryloxy, C₂-C₁₈ (hetero)arylamino, C₂-C₁₂ carboxylic acid, C₂-C₁₂ carboxylate ester, and C₁-C₁₂ alkoxy;
each Z is independently CH₂, O, or NR₄;
each f is independently an integer from 0 to 50; and
each n is independently an integer from 1 to 20.

In some embodiments, each G₁, G₂ is independently selected from the group consisting of halo (F, Cl, Br, I), C₁-C₆ alkyl, and PEG.

The water-soluble monomer may be a DHP-based monomer having a chemical structure according to Formula (III):

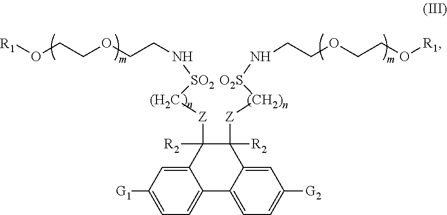

wherein each G₁, G₂ is independently halo (F, Cl, Br, I); each Z is independently selected from the group consisting of O, CH₂, and NH; each R₁ is independently alkyl (C₁-C₃); each R₂ is independently H, alkyl (C₁-C₆); each n is independently 1-6; and each m is independently 5-50. In some embodiments, G₁ and R₂ are each Br; each Z is O; each R₁ is CH₃; each R₂ is H; each n is independently 2-4; and each m is independently 5-20. In some embodiments, each n is 3; and each m is 11.

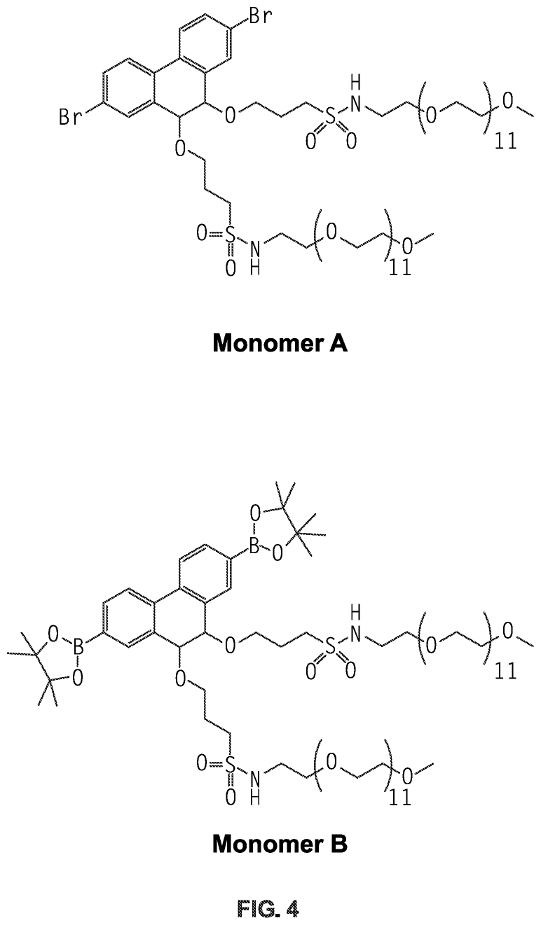

Chemical structures of specific monomer A and monomer B species are shown in FIG. 4. It was found that monomer A as shown in FIG. 4 could prevent non-specific interaction between two SN conjugates. The effective concentration was found to be in a range of 200 to 800 micrograms per test (50 uL). However, monomer B could not prevent non-specific interaction between two SN conjugates and also showed some non-specific binding on negative cells.

Without being bound by theory, it is assumed that the monomer A randomly interacts with the polymer backbone and hence prevents from the interaction between two polymer conjugates. In contrast, the protein stabilizer such as gelatin or casein due to its sticky nature likely masks these dye conjugates and prevents them from approaching in a close proximity.

### Preservatives

The aqueous DM buffer composition may include any appropriate preservative. The preservative may be an antioxidant, biocide, or antimicrobial agent. The preservative may be an inorganic salt. The preservative may be sodium azide, 2-chloroacetamide, 2-methylisothiazolinone, salicylic acid, ProClin™, Kathon™ CG, 5-chloro-2-methyl-4-isothiazolin-3-one, or 2-methyl-4-isothiazolin-3-one.

### Colorants

The aqueous DM buffer composition may include a colorant. The colorant may be a FD&C colorant. The colorant may be, for example, Allura Red (FD&C Red 40, disodium 6-hydroxy-5-[(2-methoxy-5-methyl-4-sulfonatophenyl) azo]-2-naphthalenesulfonate).

### Polymers

Fluorescent polymer dyes in different solvents were also investigated for preparation of DM buffer composition. However, more spread of the negative population compared to liquid cocktails was exhibited.

From different reagents tested, firstly a DM buffer containing trehalose dihydrate, monomer A, gelatin type B, and Empigen® BB zwitterionic surfactant was formulated.

An initial technical solution was developed in the form of a DM buffer for drying fluorescent dye conjugates to maintain integrity of dye structure, decrease aggregation. In one embodiment, a DM buffer formulation having a Gelatin and Monomer A, decreased aggregation issues during the drying process. With optimal protein stabilizer concentration in prior dry down technology and optimal concentration of monomer and gelatin, the DM assists in maintaining integrity of fluorescent dye conjugates and solves the problems associated with aggregation during drying of fluorescent dye conjugates.

The DM buffer comprises a water-soluble monomer; a protein stabilizer; a carbohydrate stabilizer; and a zwitterionic surfactant. The DM buffer may include a water-soluble monomer comprising a DHP-based monomer, and one or more, or two or more, poly(ethylene glycol) moieties. The water-soluble monomer may comprise a structure according to Formula (I). The protein stabilizer may be an albumin protein. The protein stabilizer may be a gelatin protein. The protein stabilizer may include casein protein.

Subsequently, DM buffer was further improved for preventing non-specific interaction between SuperNova™ conjugates and non-specific monocyte pull-out, which was named as “DM2” (DM 2). DM2 contains Trehalose dihydrate, Monomer A, Prionex® gelatin-Type A and DMMA surfactant.

To improve stability, DM2 was further optimized by replacing Prionex® gelatin-Type A with Casein 10× blocking buffer. This buffer was named as “DM2+S” (stabilizer). Additives present in final formulation is provided in Table 2. pH of DM2+S buffer was found to be 7 to 7.4. Dry tubes generated using DM2+S resulted in preventing non-specific interactions of the SuperNova™ conjugates as well as prevention of non-specific pullout without hindering the performance (brightness and population recruitment) of conjugates suspended in the cocktail and achieving greater stability (till yet 6-month real time stability has been established) of the dry product. In preparation of dry tubes, bulk formulation of SuperNova™ conjugates has been used.

### EXAMPLES

### Example 1A. Testing Procedures

### General Processes and Procedures Used in the Present Examples are Given Below:

Drying: The term drying as used herein refers to Vacuum drying at particular vacuum pressure for a certain number of hours.
Mixing two conjugates: Mixing of two conjugates refers to mixing the two conjugates in a 5 ml tube.
Liquid testing: The term liquid testing refers to mixing the conjugates together in a test tube (liquid cocktail) followed by staining cells using the mixed conjugates.
Dried testing: The term dried testing refers to mixing the conjugates together in a test tube and drying using vacuum drying followed by staining cells using the dried conjugates.

Testing as used herein refers to the following protocol:
1. Prepare required x number of tubes. (x tubes depend of the performance tested).
2. Add calculated volume of conjugated antibody (at required dose) to each tube or used the dried test tube for next step.
3. Add 100 μL of whole blood in each tube. Vortex the tubes gently for 6 to 8 secs.
4. Incubate for 15 to 20 minutes at room temperature (18-27° C.), protected from light.
5. Add 2 ml VersaLyse+IOTest3 Fixative mixture (2 ml Versalyse Ref. A09777+50 μl IOTest3 fixative 10× Ref. A07800) Vortex immediately to ensure proper mixing and incubate for 20 minutes at room temperature (18-27° C.), protected from light.
6. Centrifuge for 5 minutes at 300 g at room temperature.
7. Remove the supernatant by aspiration.
8. Re-suspend the cell pellet using 3 mL of 1×PBS.
9. Centrifuge for 5 minutes at 300 g at room temperature.
10. Remove the supernatant by aspiration and re-suspend the cell pellet using: 0.3 ml of 1×PBS 1× or 1×PBS+0.1% Formaldehyde (1 ml 1×PBS+12.5 μl of 10×IOTest3 fixative).

Compensation: In cytometry, compensation is a mathematical correction of a signal overlap between the channels of the emission spectra of different fluorochromes. Therefore, this correction factor was used to eliminate the bleeding of signals into other unwanted channels. Manual compensation was performed to assess the conjugate performance.

### Example 1B. Flow Cytometry Protocol

The following protocol was employed for specimen processing.

A stain lyse wash protocol was used to prepare and process the sample for flow cytometry acquisition. Samples were processed and acquired on the DxFLEX/CytoFLEX flow cytometer (Beckman Coulter, Inc.) to analyze the performance of the various formulations:
1. Add 100 μL of K2EDTA anticoagulated blood to dry stain tube or tube having liquid antibody cocktail and vortex for 6 to 8 seconds. Incubate in dark for 15 to 20 min at room temperature.
2. Add 2 mL of Versa Fix (for 1 ml Versalyse add 25 μL of IOTest 3 fixative) lysing reagent solution (Beckman Coulter, Inc.) and vortex briefly to ensure proper mixing. Incubate in dark for 20 min at room temperature.
3. After incubation with lysis & fix buffer (Versa Fix) centrifuge at 300 g for 5 mins at room temperature to pellet WBC. Aspirate and discard the supernatant and disaggregate pellet by vortexing.
4. Add 3 mL of 1×PBS and centrifuge at 300 g for 5 mins at room temperature.
5. Aspirate and discard the supernatant and resuspend the pellet in 300 μL of 1×PBS, gently vortex the tube and acquire tube on DxFLEX flow cytometer on recommended setting.

### Instrument Setup:

The flow cytometer instrument set up was performed using the CytoFLEX or DxFLEX Daily QC fluorospheres (Beckman Coulter, Inc.). The QC beads are used to perform daily quality control management of the instrument and identify the target values of gain. The recommended settings for gain identified by the QC protocol was used for the acquisition of the sample tubes in the flow cytometer.

Compensation was setup for 12 colors using the universal compensation kit (Beckman Coulter, Inc.) and singles of SN polymer dye conjugates in liquid and/or dry format.

### Example 2. Dry Mix Component Selection

An aqueous DM buffer formulation was developed for drying fluorescent dye conjugates. Starting with prior drying technology, several reagents (Table 1) were tested for preventing non-specific interaction as well as non-specific binding of polymer dye antibody conjugates. Water or PBS was used in preparation of stock solutions of these reagents. The reagents tested and outcome for each reagent are shown in Table 1. Reagents shown in bold typeface were further evaluated.

TABLE 1
Reagents screened for preventing non-specific interaction and non-
specific binding of polymer dye antibody conjugates

| Sr. | | |
|---|---|---|
| No. | Reagents/components | Outcome |
| 1 | BSA | Could not completely prevent the polymer dyes |
| | | from interactions. |
| 2 | PEG550 | Did not dry and hence stability was concern. |
| 3 | BSA-ox | Could not completely prevent the polymer dyes |
| | | from interactions. |
| 4 | Empigen ® BB detergent | Could prevent the non-specific binding on |
| | | monocytes and granulocytes but was not able to |
| | | prevent polymer interactions. |
| 5 | N-Lauryl sarcosine | Could prevent the non-specific binding on |
| | sodium salt | monocytes and granulocytes but was not able to |
| | | prevent polymer interactions. |
| 6 | Monomer-A | 1. Could prevent non-specific interaction between |
| | | two SN conjugates. |
| | | 2. Effective concentration of monomer was found |
| | | to be in a range of 200 μg to 800 μg per test. |
| 7 | Monomer- B | Could not prevent non-specific interaction |
| | | between two SN conjugates and also had showed |
| | | some non-specific binding. |
| 8 | Polymer in different | Impacted the negative population by binding non- |
| | solvents (Inhouse product | specifically. |
| | of Beckman) | |
| 9 | Gelatin- type A | More spread of the negative population compared |
| | | to liquid cocktails. |
| 10 | Gelatin- type B | 1. Was able to prevent the non-specific |
| | | interactions but the preparation of stocks was |
| | | difficult. |
| | | 2. Effective concentration was found to be in a |
| | | range of 150 μg to 450 μg per test. |
| 11 | Casein powder | Was able to prevent non-specific interaction |
| | | between polymers at a range of 0.15% to 0.3%. |
| | | Preparation of stock solution of casein was |
| | | difficult due to solubility issues. |
| 11 | Lignosulfonic acid | Negative background of SN v605 and SN v786 |
| | | population increased compared to Dry Mix when |
| | | LSA added. |
| 12 | Carragenan | Preparation of stock is challenging and requires |
| | | special care, due to addition of this additive non |
| | | specific interactions increased. |
| 13 | Sodium Alginate | Preparation of stock is challenging and require |
| | | special care, overall spread of granulocytes, |
| | | monocytes and lymphocytes was higher. |
| 14 | Casein Blocking Buffer | Casein at 4× (2 times diluted), 2.5× (4 times |
| | 10× | diluted) 1× (9 times diluted) concentration could |
| | | effectively prevent interaction between two |
| | | polymer conjugates. |
| 15 | Prionex ® Highly | 1. At concentration equivalent to Gelatin-B, |
| | purified Type A gelatin | Prionex ® was proved efficient in preventing non- |
| | | specific interaction, but the stability of gelatin was |
| | | less. |
| | | 2. Protein concentration of ready-made solution of |
| | | gelatin (Prionex ®) was quantified and based on |
| | | that the effective concentration was found to be |
| | | approximately in range of 67 μg to 135 μg per test. |
| 16 | Gelatin- cold water fish | 1. In terms of performance, it was equivalent to |
| | | Gelatin type B. |
| | | 2. Effective concentration was found to be in a |
| | | range of 150 μg to 450 μg per test. |
| 17 | L-Ascorbic acid | Non-specific monocyte pull out with SN v605 |
| | | conjugate were less at 0.2 mM and 0.6 mM |
| | | concentration. 0.6 mM concentration had less |
| | | spread in SN v786 positive population in V610 |
| | | channel. Did not have additional impact in |
| | | containing the spread of the population. |
| 18 | Citric Acid | Reduced degranulation of granulocytes was |
| | | observed. Non-specific monocyte pull out in SN |
| | | v605 conjugate was less at all the concentrations |
| | | (0.1 to 0.6 mM). Did not have additional impact in |
| | | containing the spread of the population. |
| 19 | Polysorbate 80 | Nonspecific monocyte pull out and spread of the |
| | | population with SN v605 conjugate was contained |
| | | at 0.015% and 0.075%. |
| 20 | Acrylamide | Shows more spread with SN v786 positive |
| | | population in V610 channel. |
| 21 | Trehalose- decanoate | At dose equivalent to trehalose (400 μg), the |
| | | compound lyse all the cells completely. Lowering |
| | | the dose would compromise dry down of |
| | | conjugates. |
| 22 | Trehalose- tetra decanoate | At dose equivalent to trehalose (400 μg), the |
| | | compound lyse all the cells completely. Lowering |
| | | the dose would compromise dry down of |
| | | conjugates. |
| 23 | Trehalose- hexa decanoate | At dose equivalent to trehalose (400 μg), the |
| | | compound lyse all the cells completely. Lowering |
| | | the dose would compromise dry down of |
| | | conjugates. |
| 24 | 3-[N,N-Dimethyl(3- | 1. DMPA precipitates at room temperature in the |
| | palmitoylamino- | aqueous solution but works functionally equivalent |
| | propyl)ammonio]- | to Empigen. |
| | propanesulfonate | 2. Effective concentration was found to be in a |
| | (DMPA) | range of 0.002% to 0.006%. |
| 25 | 3-(N,N- | 1. DMMA worked functionally equivalent to |
| | Dimethylmyristylam- | Empigen, therefore was considered for further |
| | monio)propanesulfonate | testing. |
| | (DMMA) | 2. Effective concentration was found to be in a |
| | | range of 0.004% to 0.018% |

The following DM candidate reagents were used in dry down of polymer dye conjugate tubes: BSA (bovine serum albumin), PEG550 (poly(ethylene glycol) methyl ether, avg Mₙ 550), BSA-ox (oxidized BSA), Empigen® BB detergent (N-(alkyl C₁₀-C₁₆)—N,N-dimethylglycine betaine), N-lauryl sarcosine sodium salt, monomer A, monomer B, polymer in different solvents, Gelatin-type A, Gelatin-type B, lignosulfonic acid, carrageenan, sodium alginate, casein blocking buffer 10×, Prionex® highly purified type A gelatin, gelatin-cold water fish, L-ascorbic acid, citric acid, polysorbate 80, acrylamide, trehalose decanoate, trehalose-tetra decanoate, trehalose-hexa decanoate, 3-[N,N-Dimethyl (3-palmitoylaminopropyl) ammonio]-propanesulfonate (DMPA), and 3-(N,N-Dimethylmyristylammonio) propanesulfonate (DMMA). The chemical structures of monomer A and monomer B are shown in FIG. 4. Monomer A may be 3,3′-((2,7-dibromo-9,10-dihydrophenanthrene-9,10-diyl)bis(oxy)) bis(N-(2,5,8,11,14,17,20,23,26,29,32,35-dodecaoxaheptatriacontan-37-yl) propane-1-sulfonamide). The dry down tubes were tested according to protocol of Example 1. Results of testing are shown in Table 1. Empigen®, monomer A, gelatin-type B, casein blocking buffer 10×, Prionex® highly purified Type A gelatin, gelatin cold water fish, DMPA, and DMMA were selected for further testing.

From different reagents tested, a first DM (DM) buffer formulation was developed containing Trehalose dihydrate, Monomer A, Gelatin type B, and Empigen BB® detergent (N,N-dimethyl-N-dodecylglycine betaine; N-(alkyl C₁₀-C₁₆)—N,N-dimethylglycine betaine).

Subsequently, an improved DM buffer formulation was developed for preventing non-specific interaction between SuperNova™ conjugates and non-specific monocyte pull-out which was named as DM2 (DM 2). DM2 contains Trehalose dihydrate, Monomer A, Prionex® gelatin and DMMA. However, later it was found out that dry tubes prepared using DM2 has stability issue. Eventually, DM2 was further optimized by replacing Prionex with Casein 10× blocking buffer. This buffer was named as DM2+S (stabilizer).

Additives present in final DM buffer formulation are provided in Table 2. The pH of DM2+S buffer was found to be 7 to 7.4. Dry tubes generated using DM buffer DM2+S resulted in preventing non-specific interactions of the SuperNova™ conjugates as well as prevention of non-specific pullout without hindering the performance (brightness and population recruitment) of conjugates suspended in the cocktail and achieving greater stability (so far 6-month real time stability has been established) of the dry product. In preparation of dry tubes, a bulk formulation of SuperNova™ conjugates has been used.

TABLE 2
Components of representative DM Formulation

| Sr. | Additives required | Part | |
|---|---|---|---|
| No. | to make DM Buffer | Number | Supplier |
| 1 | Casein 10× blocking buffer | B6429 | Sigma |
| | | | Aldrich |
| 2 | Trehalose dihydrate | T9531 | Sigma |
| | | | Aldrich |
| 3 | DMMA | T7763 | Sigma |
| | (3-(N,N-Dimethylmyristylammonio) | | Aldrich |
| | propanesulfonate) | | |
| 4 | Monomer A | C37603 | Lumigen |
| 5 | Sodium Azide | 10199-00 | Sigma |
| | | | Aldrich |
| 6 | Allura Red AC azo dye | 458848 | Sigma |
| | | | Aldrich |

TABLE 3
Components of representative DM formulation, amount per test

| | | Amount, μg/test |
|---|---|---|
| | Amount, μg/test | (Standardized |
| Components | (Active range) | dose) |
| Trehalose | 2000-3000 | 2500 |
| Monomer A | 200-800 | 400 |
| Casein | 8.4-72 | 17.82 |
| DMMA | 2-15 | 9 |
| Allura red | 5-10 | 9 |
| Sodium azide | 2-10 | 5 |

Table 3 illustrates preferred amounts of components of DM2+S DM buffer per test. In some embodiments, the amounts are per 28.48 microliters buffer without dye conjugates. In some embodiments, the amounts are per 50 microliters buffer with dye conjugates.

In some embodiments, the DM buffer may comprise trehalose, monomer A, DMMA, and casein. In some embodiments, the dry down buffer may comprise appropriate concentrations to obtain 2000-3000 micrograms per test trehalose; 200-800 micrograms per test Monomer A; 8.4-72 micrograms per test casein; and 2-15 micrograms per test DMMA. In some embodiments, the DM buffer is an aqueous buffer that may include 70 to 105 mg/mL, or 80 to 100 mg/mL trehalose dihydrate; 7 to 28 mg/mL, or 10 to 20 mg/mL monomer A; 0.07 to 0.53 mg/mL, or 0.2 to 0.4 mg/mL DMMA, and 0.3 to 2.5 mg/mL, or 0.5 to 0.8 mg/mL casein, without dye conjugates. The DM buffer may be prepared from stock concentrations of carbohydrate stabilizer, water-soluble monomer, zwitterionic surfactant, and protein stabilizer, for example, in water or a PBS buffer. For example, the stock concentrations may comprise about 400 mg/ml trehalose dihydrate, about 40 mg/ml monomer A, about 1.5 mg/ml DMMA, and or about 2-20 mg/mL casein.

### Example 3. Design of Experiments for DM Buffer & their Outcomes

In order to achieve optimal results, concentration of each component of the DM Buffer (except Trehalose dihydrate, sodium azide and Allura Red) were titrated and optimized.

### 1. Trehalose Dihydrate

Trehalose dihydrate was one of the components of prior technology. In absence of this additive, it was difficult to dry down conjugates and other buffer components.

### 2. Titration of Casein 10× Blocking Buffer

### 2.1: Experiment: 1

Aim: To find out optimum concentration of casein in preventing non-specific binding in liquid format.
Method: In this experiment, different concentrations (5×, 2.5×, 1.25×, 0.625× and 0.31×) of ready to use solution of casein 10× blocking buffer were tested according to protocol of Example 1.

### Results and Observations

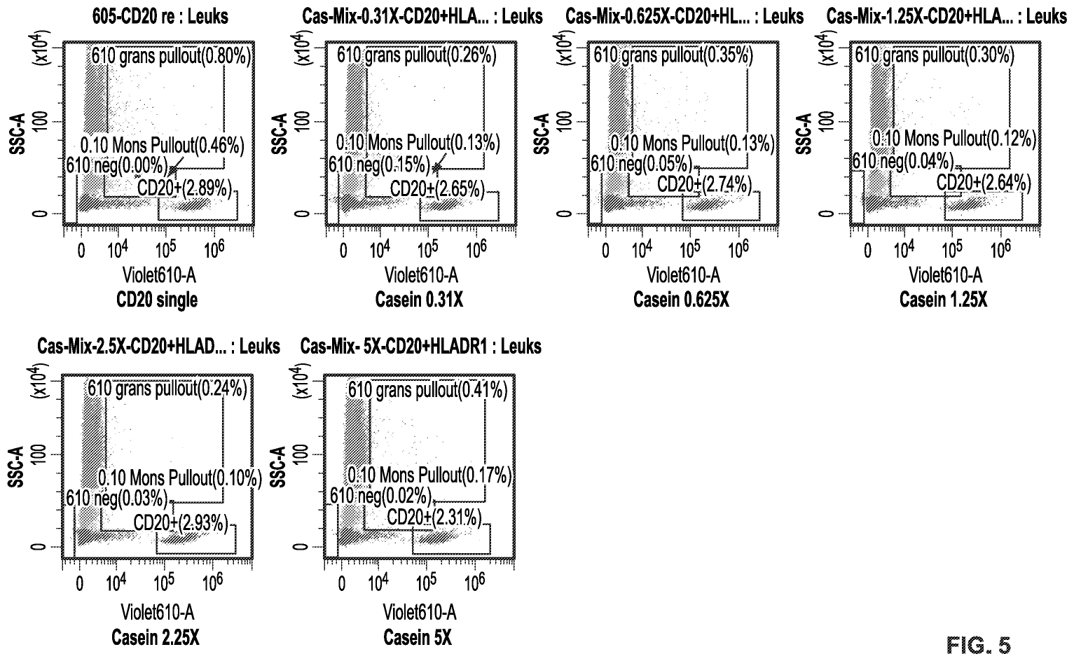

FIG. 5 shows Dose titration of Casein and comparative CD20-SNv605 flow plots with negative control (no casein), 0.31×, 0.625×, 1.25×, 2.25× and 5× casein. Casein at 1.25×, 2.5× and 5× concentration helps in reduction of non-specific monocyte pull-out in V610 channel. In addition, % recruitment of CD20+ cells were same across all the formulation when compared with CD20 singles (without casein). % recruitment of HLADR+ cells was also found to be consistent in all the groups (data not shown). Hence, casein alone was evaluated further in dry tubes at different concentration. Concentration of casein was further optimized to find out the optimum concentration for dry tubes.

### 2.2: Experiment: 2

Aim: To find out optimum concentration of casein in preventing non-specific binding in dry format.
Method: In this experiment, different concentration (4×, 2.5× and 1×) of ready to use solution of casein 10× blocking buffer was used to dry 12 different antibody conjugates along with other additives in place. 2.5× is the standardized concentration. Hence, 2.5× concentration was taken as a reference to compare with 1× and 4× concentrations. Here, two different lots (L¹ and L²) of casein 10× blocking buffer were tested.

### Results and Observations:

Overlays of dual fluorescence plots (V610 vs V780, PB450 vs V610 and PB450 vs V780) of DM buffer with different concentrations (4×, 2.5× and 1×) were compared. (data not shown). Overlays for dual fluorescence plots for SuperNova™ (Violet channel) vs conventional channels and classical vs classical conjugates channels across different concentrations were also compared (data not shown). All these comparisons confirmed that there is no significant variation across different concentrations of casein tested. Table 4 and Table 5 show comparison across different concentrations of casein for absolute % delta recruitment and MdFI for all the specificities.

TABLE 4
Absolute % Delta Recruitment for all 12C specificities for 1X and
4X Casein concentration as compared to 2.5X

| | | | | Average 3 | | | | Average 3 |
|---|---|---|---|---|---|---|---|---|
| | | | | donors | | | | donors (L1 |
| | L1 1X-L1 2.5 X | | | (L1 1X- | L1 4X-L1 2.5X | | | 4X-L1 |
| | D5 | D14 | D16 | L1 2.5 X) | D5 | D14 | D16 | 2.5X) |
| CD45+ Lymph % | 0.66% | 0.21% | 2.30% | 1.06% | 0.22% | 1.21% | 1.34% | 0.92% |
| Parent | | | | | | | | |
| CD4 Mono % Parent | 0.31% | 0.10% | 0.47% | 0.29% | 0.13% | 0.29% | 0.80% | 0.41% |
| CD4 Lymphs % Parent | 0.15% | 1.16% | 2.59% | 1.30% | 0.14% | 0.19% | 0.74% | 0.36% |
| CD20+ % Parent | 0.46% | 0.02% | 0.38% | 0.29% | 0.28% | 0.21% | 0.43% | 0.31% |
| CD16+ % Parent | 1.79% | 0.91% | 2.84% | 1.85% | 0.60% | 0.13% | 1.87% | 0.87% |
| CD16+ Lymphs % | 0.00% | 0.29% | 0.05% | 0.11% | 0.06% | 0.18% | 0.03% | 0.09% |
| Parent | | | | | | | | |
| CD25+ % Parent | 0.31% | 0.23% | 1.58% | 0.71% | 0.28% | 0.13% | 0.02% | 0.14% |
| CD3+ % Parent | 0.08% | 0.89% | 3.05% | 1.34% | 0.01% | 0.43% | 0.76% | 0.40% |
| CD38+ % Parent | 0.36% | 1.37% | 2.83% | 1.52% | 0.93% | 0.14% | 0.28% | 0.45% |
| CD38+ Mons % Parent | 0.26% | 0.81% | 0.10% | 0.39% | 0.23% | 0.82% | 0.19% | 0.41% |
| CD27+ % Parent | 0.04% | 0.88% | 3.03% | 1.32% | 0.16% | 0.47% | 0.54% | 0.39% |
| CD10+ % Parent | 1.31% | 0.06% | 2.38% | 1.25% | 0.14% | 0.97% | 1.96% | 1.02% |
| CD14+ % Parent | 0.06% | 0.42% | 0.15% | 0.21% | 0.09% | 0.50% | 0.69% | 0.43% |
| CD56 Lymphs Median | 0.03% | 0.20% | 0.06% | 0.10% | 0.06% | 0.54% | 0.26% | 0.29% |
| PB450-A | | | | | | | | |
| CD8+ % Parent | 0.08% | 0.45% | 0.43% | 0.32% | 0.08% | 0.78% | 0.05% | 0.30% |

TABLE 5
MdFI comparison for all 12C specificities for 1X and 4X Casein
concentration as compared to 2.5X

| | | | | Average 3 | | | | Average |
|---|---|---|---|---|---|---|---|---|
| | | | | donors | | | | 3 donors |
| | | | | (L1 | | | | (LI |
| | L1 1X/L1 2.5X | | | 1X/L1 | L1 4x/L1 2.5X | | | 4x/L1 |
| | D5 | D14 | D16 | 2.5X) | D5 | D14 | D16 | 2.5X) |
| CD45+ Lymph | 101.20% | 95.83% | 98.60% | 98.54% | 100.98% | 100.69% | 95.98% | 99.22% |
| Median APC- | | | | | | | | |
| A750-A | | | | | | | | |
| CD4 Mono | 109.28% | 117.64% | 73.30% | 100.74% | 110.66% | 90.34% | 100.80% | 100.60% |
| Median | | | | | | | | |
| Violet780-A | | | | | | | | |
| CD4 Lymphs | 99.00% | 103.01% | 104.82% | 102.27% | 99.28% | 97.87% | 100.71% | 99.29% |
| Median | | | | | | | | |
| Violet780-A | | | | | | | | |
| CD20+ Median | 100.28% | 101.75% | 107.97% | 103.33% | 99.12% | 97.69% | 98.14% | 98.32% |
| Violet610-A | | | | | | | | |
| CD16+ Median | 100.84% | 97.84% | 101.39% | 100.02% | 101.69% | 101.52% | 99.20% | 100.80% |
| FITC-A | | | | | | | | |
| CD16+ Lymphs | 95.30% | 84.54% | 85.44% | 88.43% | 98.09% | 91.49% | 99.40% | 96.33% |
| Median | | | | | | | | |
| FITC-A | | | | | | | | |
| CD25+ Median | 103.49% | 102.12% | 95.01% | 100.21% | 98.17% | 89.32% | 98.03% | 95.17% |
| PE-A | | | | | | | | |
| CD3+ Median | 105.85% | 105.62% | 106.14% | 105.87% | 94.11% | 82.06% | 94.08% | 90.08% |
| ECD-A | | | | | | | | |
| CD38+ Median | 99.35% | 101.27% | 97.58% | 99.40% | 91.24% | 79.19% | 88.14% | 86.19% |
| PC5.5-A | | | | | | | | |
| CD38+ Mons | 89.47% | 97.59% | 106.81% | 97.96% | 83.83% | 91.49% | 89.31% | 88.21% |
| Median | | | | | | | | |
| PC5.5-A | | | | | | | | |
| CD27+ Median | 107.75% | 106.68% | 109.06% | 107.83% | 87.87% | 77.11% | 87.23% | 84.07% |
| PC7-A | | | | | | | | |
| CD10+ Median | 103.25% | 100.34% | 101.26% | 101.62% | 97.67% | 89.50% | 93.20% | 93.46% |
| APC-A | | | | | | | | |
| CD14+ Median | 106.58% | 104.42% | 103.52% | 104.84% | 98.14% | 100.24% | 99.59% | 99.32% |
| APC-A700-A | | | | | | | | |
| CD56 Lymphs | 108.79% | 100.41% | 107.97% | 105.72% | 119.61% | 104.84% | 105.16% | 109.87% |
| Median | | | | | | | | |
| PB450-A | | | | | | | | |
| CD8+ Median | 100.23% | 102.58% | 100.42% | 101.08% | 101.93% | 97.67% | 99.97% | 99.86% |
| KO525-A | | | | | | | | |

This example demonstrates that overlay plots, MdFI, and absolute % delta recruitment have no significant variation at different concentrations (1×, 2.5×, 4×) of Casein 10× blocking buffer. Absolute % delta recruitment was found to be <5% between lots and across different concentrations tested, attributing to minimal/no effect of different concentrations of Casein 10× blocking buffer. Therefore, 1× to 4× of Casein blocking buffer can be used for drying SuperNova™ conjugates along with other conventional dyes.

### Titration of Monomer A:

### 3.1: Experiment: 1

Aim: To titrate the Monomer A concentration for dry down of two polymer dye antibody conjugates.
Method: Monomer concentrations of 50 μg, 100 μg, 200 μg, 400 μg and 800 μg micrograms per test were tested in a formulation consisting of CD45-FITC, CD20-SN-v605 and CD3-SN-v786 in dry format along with other additives kept constant.
Results and observations: two-dimensional flow cytometer fluorescence plots of CD20-SN v605 and CD3-SN v786 with various doses of Monomer A were analyzed (data not shown). From different doses of monomer, 200 μg and 400 μg of monomer was sufficient to prevent the interaction of polymer dye conjugates.

### 3.2: Experiment: 2

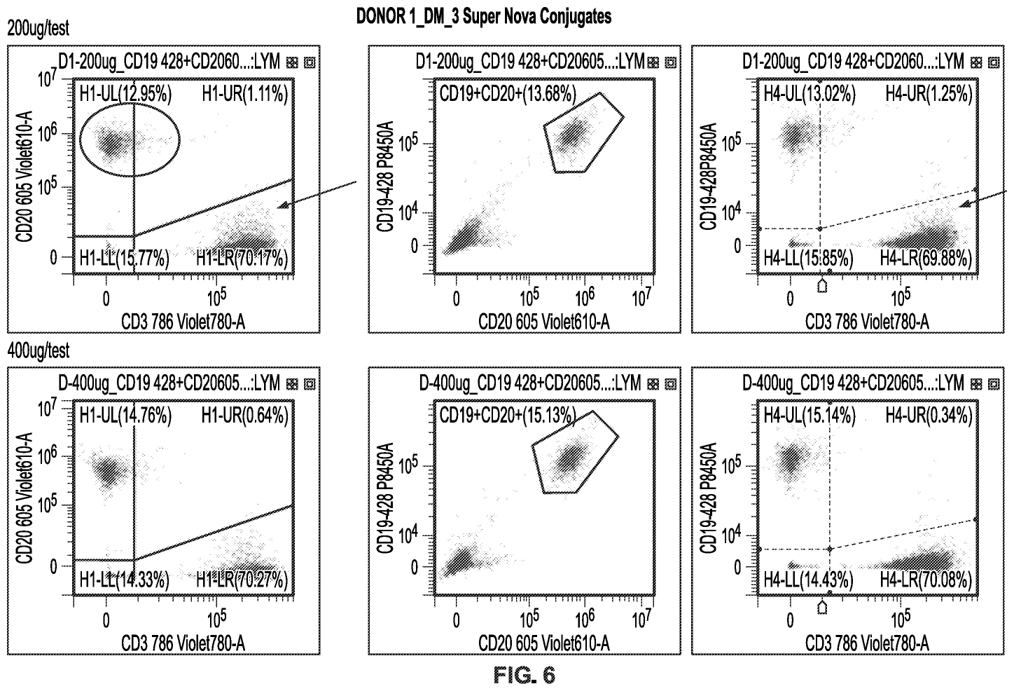

Aim: To titrate the Monomer A concentration for dry down of three polymer dye antibody conjugates.
Method: Monomer A concentrations of 200 micrograms per test and 400 micrograms per test were tested in a formulation consisting of CD45-FITC, CD19-SN v428, CD20-SNv605 and CD3-SNv786 in dry format along with other additives kept constant.
Results and observations: FIG. 6 shows dual fluorescence plot of dry formulation for 3 color tested for 200 μg (top row) and 400 μg per test (bottom row) of monomer concentration. Between 200 μg and 400 μg of monomer A, tubes containing 400 μg showed better results in terms of spread of the population from one channel spilling into other channels (as indicated by arrows and circle in FIG. 6), and was enough to prevent the interaction of polymer dye antibody conjugates.

### 4. Titration of DMMA

To evaluate the role of DMMA (3-(N,N-Dimethylmyristylammonio) propanesulfonate) in preventing non-specific interaction.

### 4.1: Experiment 1: Dose Titration of Zwitterionic Surfactant DMMA in the Presence of Two SuperNova™ Polymer Dye Antibody Conjugates

Aim: To find out optimum concentration of DMMA in preventing non-specific interaction between two SuperNova™ conjugates.
Method: In this experiment, different concentrations of DMMA (Sigma, part no. T7763-5G) were tested by replacing Empigen® BB with DMMA in DM version 1. DMMA is a water soluble zwitterionic surfactant. DMMA was tested in order to find out better option for Empigen® in reducing non-specific interaction with easy manufacturability. Different concentrations of DMMA along with details of groups tested are provided in Table 6.

TABLE 6
Evaluation of different concentration of DMMA in dry formulation

| | | | | | Vol. | | | | Vol. of | | |
|---|---|---|---|---|---|---|---|---|---|---|---|
| | Sample | Vol. | Vol. | Vol. | Conjugates, | Vol. | Vol. | Vol. | CD45- | | |
| Ser. | with | Gelatin, | Trehalose, | monomer, | HLADR, | CD20, | empigen, | additive, | AA750, | | Tot. |
| No. | additive | ul | ul | ul | ul | ul | ul | ul | ul | PBS | vol. |
| 1 | DMMA | 6 | 6.25 | 10 | 0.95 | 3.38 | 0 | 1.25 | 2.75 | 19.42 | 50 |
| | 0.00375% | | | | | | | | | | |
| 2 | DMMA | 6 | 6.25 | 10 | 0.95 | 3.38 | 0 | 2.5 | 2.75 | 18.17 | 50 |
| | 0.0075% | | | | | | | | | | |
| 3 | DMMA | 6 | 6.25 | 10 | 0.95 | 3.38 | 0 | 5 | 2.75 | 15.67 | 50 |
| | 0.015% | | | | | | | | | | |
| 4 | DMMA | 6 | 6.25 | 10 | 0.95 | 3.38 | 0 | 10 | 2.75 | 10.67 | 50 |
| | 0.03% | | | | | | | | | | |
| 5 | DM | 6 | 6.25 | 10 | 0.95 | 3.38 | 5 | 0 | 2.75 | 15.67 | 50 |

Here, different concentration of DMMA, i.e., 0.03%, 0.015%, 0.0075% and 0.00375% were tested in liquid and dry tubes and compared with DM buffer. Concentrations of DMMA were selected on the basis of Empigen BB® concentration (0.15%) which is one of the components of DM buffer. Appropriate controls were used in the experiment. 0.15% DMMA was added in the final formulation to attain respective concentration of DMMA as mentioned in Table 6. Effect of DMMA was tested using two Supernova™ conjugates, CD20-SN-v605 and HLADR-SN-v786. Specimens were processed using protocol per Example 1B. Experiment was carried out on 2 donors.

Results and observations: Flow plots of scatter properties for CD20-SN-v605, HLADR-SN-v786 and dual positive population were compared for all the dry DMMA formulation tested. (data not shown). DMMA is a surfactant and may cause cell death at higher concentration. Overlay scatter plots of DM vs each of the concentration of DMMA tested were analyzed. DMMA at 0.03% caused cell death and not evaluated for other parameters. DMMA at 0.0075% and 0.00375% showed comparable scatter properties with DM. Similarly, % recruitment of CD20+ and HLADR+ found to be similar in DMMA at 0.0075% and 00375% compared to respective liquid singles. In addition, DMMA at 0.0075% and 0.00375% showed less spread of 786+ population in 610 channels as compared to DM.

This example demonstrates that DMMA at 0.0075% and 0.00375% shows comparable scatter properties, % recruitment of CD20+ and HLADR+ population with DM. Concentration of DMMA was further optimized within a range of 0.0075% to 0.00375% to obtain optimum concentration.

### 4.2: Experiment 2: Dose Optimization of DMMA in the Presence of Two Supernova™ Conjugates

Aim: To find out optimum concentration of DMMA in preventing non-specific interaction between two SuperNova™ conjugates.
Method: In this experiment, concentration of DMMA was further optimized between 0.03% and 0.004% to find out optimum concentration. Different concentration of DMMA along with details of groups tested provided in Table 7.

TABLE 7
Optimization of DMMA concentration in dry formulation

| | | | | | | | | | Vol. | | |
|---|---|---|---|---|---|---|---|---|---|---|---|
| | Sample | Vol | Vol. | Vol. | Vol. | Vol. | Vol. | Vol. | CD45- | | |
| Ser. | with | Gelatin, | Trehalose, | monomer, | HLADR, | CD20, | empigen, | additive, | AA750, | | Tot |
| No. | additive | ul | ul | ul | ul | ul | ul | ul | ul | PBS | vol. |
| 1 | DMMA | 6 | 6.25 | 10 | 0.95 | 3.33 | 0 | 10 | 2.75 | 10.72 | 50 |
| | 0.03% | | | | | | | | | | |
| 2 | DMMA | 6 | 6.25 | 10 | 0.95 | 3.33 | 0 | 7.15 | 2.75 | 13.57 | 50 |
| | 0.021% | | | | | | | | | | |
| 3 | DMMA | 6 | 6.25 | 10 | 0.95 | 3.33 | 0 | 5 | 2.75 | 15.72 | 50 |
| | 0.018% | | | | | | | | | | |
| 4 | DMMA | 6 | 6.25 | 10 | 0.95 | 3.33 | 0 | 2.67 | 2.75 | 18.05 | 50 |
| | 0.008% | | | | | | | | | | |
| 5 | DMMA | 6 | 6.25 | 10 | 0.95 | 3.33 | 0 | 1.34 | 2.75 | 19.38 | 50 |
| | 0.004% | | | | | | | | | | |
| 6 | DM | 6 | 6.25 | 10 | 0.95 | 3.33 | 5 | 0 | 2.75 | 15.72 | 50 |

Here, different concentration of DMMA, i.e., 0.03%, 0.021%, 0.018%, 0.008% and 0.004% were tested in dry tubes and compared with DM. Appropriate controls were used in the experiment. 0.15% DMMA was added in the final formulation to attain respective concentration of DMMA as mentioned in Table 7. Effect of DMMA was tested using two Supernova™ conjugates, CD20-SN-v605 and HLADR-SN-v786. Specimen were processed using protocol as mentioned in Example 1B. Experiment was carried out on 2 donors.

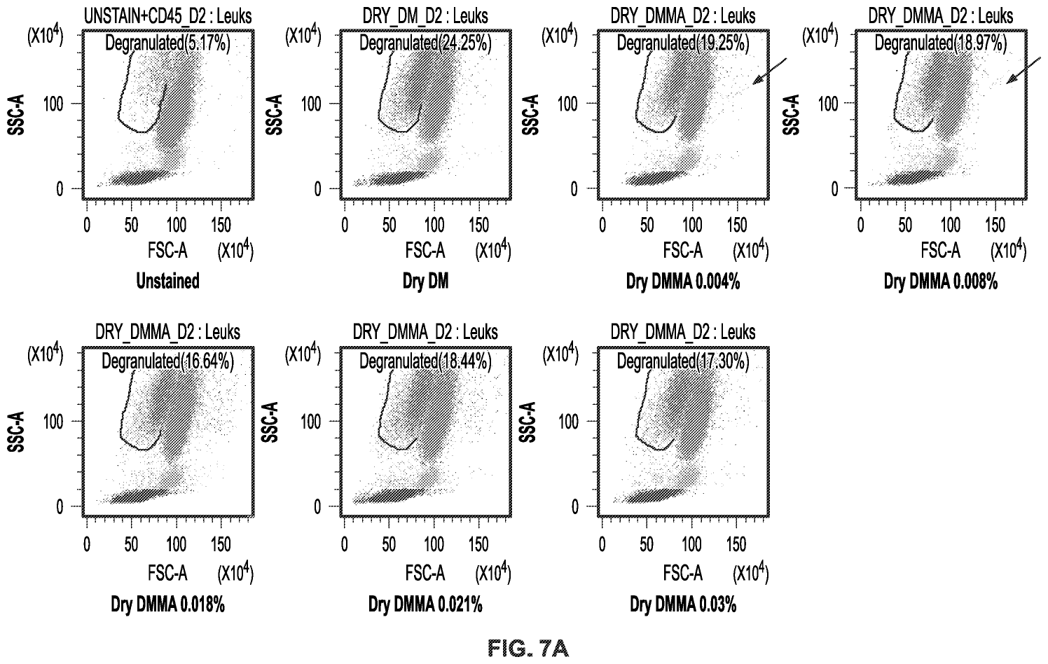

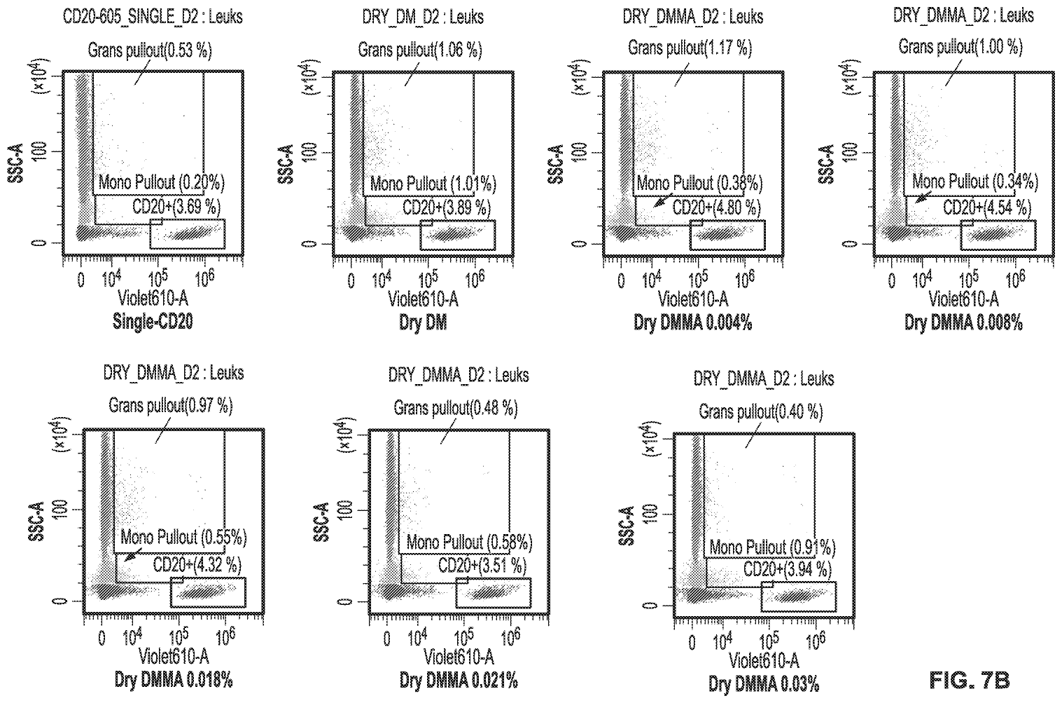

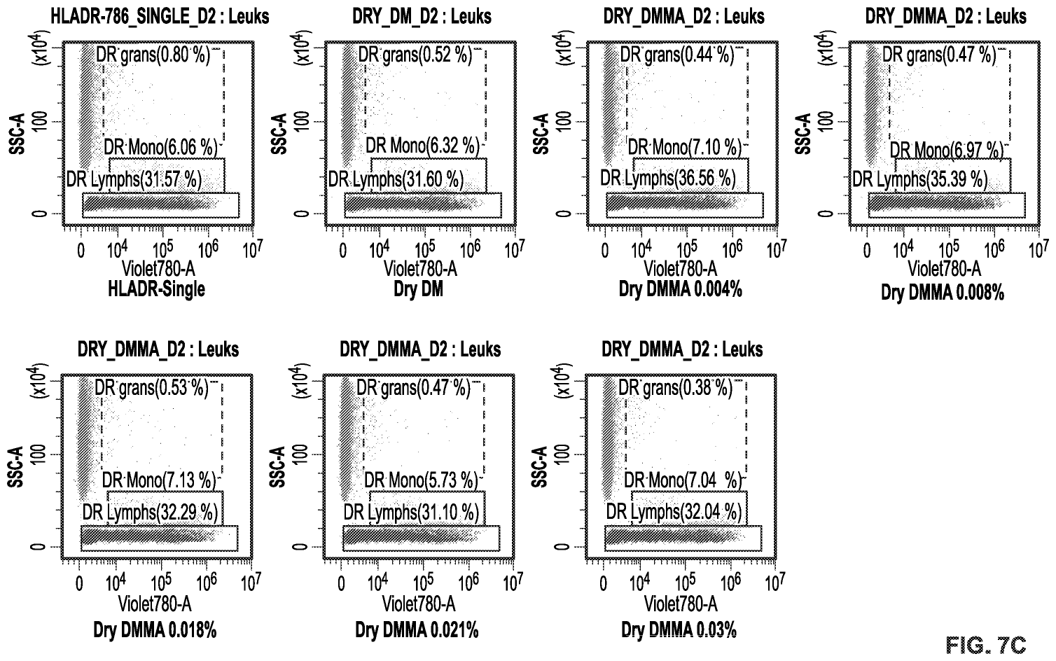

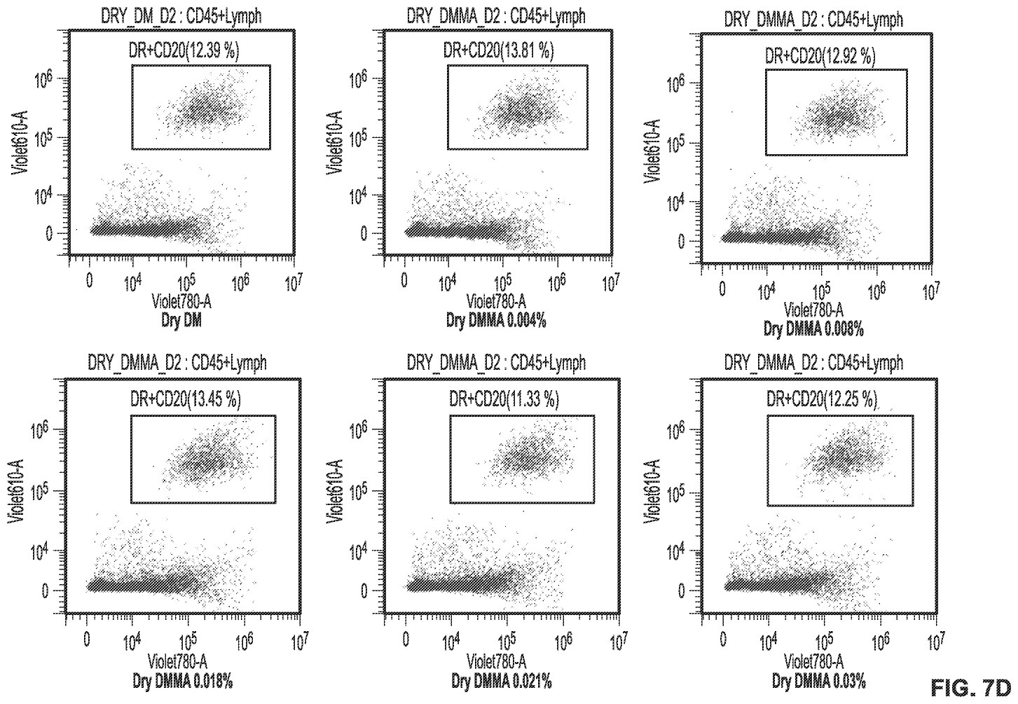

FIG. 7A-D shows flow plots of scatter properties, CD20-SN-v605, HLADR-SN-v786 and dual positive population for all the dry DMMA formulation tested. FIG. 7A shows scatter plots of all the concentration of DMMA tested along with unstained and DM. DMMA at 0.004% and 0.008% shows less non-specific neutrophil pullout (indicated by arrow) as compared to other DMMA concentrations. Similarly, DMMA at 0.004%, 0.008% and 0.018% shows less non-specific monocyte pullout in V610 channel (indicated with arrow, FIG. 7B) as compared to DM and other DMMA concentrations. % recruitment of CD20+ and HLADR+ found to be similar in all the DMMA concentrations compared to respective liquid singles. Similarly, % recruitment of dual positive population in all the DMMA concentration found to be similar as compared to DM. In addition, DMMA at 0.004%, 0.008% and 0.018% shows similar spread of 786+ events in V610 channel as compared to DM. This example demonstrates that DMMA at 0.004%, 0.008% and 0.018% shows better performance as compared to DM. Hence, these DMMA concentration were further evaluated in combination with other selected additives.

### 5. To Evaluate Performance of Different Combinations of Selected Additives

### 5.1: Experiment 1: Performance Evaluation of Combinations of Selected Additives

Aim: To evaluate performance of combination of selected additives in preventing non-specific binding and non-specific interaction between SN conjugates.
Method: In this experiment, combinations of selected additives such as DMMA+casein, DMMA+Prionex were evaluated. Different combinations of additives along with details of groups tested provided in Table 8.

TABLE 8
Combinations of additives-Two color experiment

| | | | | | | Vol. | Vol. | | | | Vol. | | |
|---|---|---|---|---|---|---|---|---|---|---|---|---|---|
| | | Gel- | Tre- | mono- | | of | of | | | | CD45- | | |
| Ser. | Sample | atin, | halose, | mer, | HLADR, | CD20, | empigen, | | | | AA750, | | Tot. |
| No. | with additive | ul | ul | ul | ul | ul | ul | DMMA | CASEIN | PRIONEX | ul | PBS | vol. |
| 1 | DM | 6 | 6.25 | 10 | 0.95 | 3.38 | 5 | 0 | 0 | 0 | 2.75 | 15.67 | 50 |
| 2 | DMMA_0.018% | 6 | 6.25 | 10 | 0.95 | 3.38 | 0 | 5 | 0 | 0 | 2.75 | 15.67 | 50 |
| 3 | DMMA_0.008% | 6 | 6.25 | 10 | 0.95 | 3.38 | 0 | 2.67 | 0 | 0 | 2.75 | 18 | 50 |
| 4 | CASEIN_2.5X | 0 | 6.25 | 10 | 0.95 | 3.38 | 5 | 0 | 6 | 0 | 2.75 | 15.67 | 50 |
| 5 | PRIONEX_2 DIL | 0 | 6.25 | 10 | 0.95 | 3.38 | 5 | 0 | 0 | 6 | 2.75 | 15.67 | 50 |
| 6 | PRIONEX_3 DIL | 0 | 6.25 | 10 | 0.95 | 3.38 | 5 | 0 | 0 | 6 | 2.75 | 15.67 | 50 |
| 7 | DMMA_0.00375 + | 0 | 6.25 | 10 | 0.95 | 3.38 | 0 | 5 | 6 | 0 | 2.75 | 15.67 | 50 |
| | CASEIN_2.5X | | | | | | | | | | | | |
| 8 | DMMA_0.018% + | 0 | 6.25 | 10 | 0.95 | 3.38 | 0 | 5 | 0 | 6 | 2.75 | 15.67 | 50 |
| | PRIONEX_2 DIL | | | | | | | | | | | | |
| 9 | DMMA_0.018% + | 0 | 6.25 | 10 | 0.95 | 3.38 | 0 | 5 | 0 | 6 | 2.75 | 15.67 | 50 |
| | PRIONEX_3 DIL | | | | | | | | | | | | |
| 10 | DMMA_0.008% + | 0 | 6.25 | 10 | 0.95 | 3.38 | 0 | 2.67 | 6 | 0 | 2.75 | 18 | 50 |
| | CASEIN_2.5X | | | | | | | | | | | | |
| 11 | DMMA_0.008% + | 0 | 6.25 | 10 | 0.95 | 3.38 | 0 | 2.67 | 0 | 6 | 2.75 | 18 | 50 |
| | PRIONEX_2 DIL | | | | | | | | | | | | |
| 12 | DMMA_0.008% + | 0 | 6.25 | 10 | 0.95 | 3.38 | 0 | 2.67 | 0 | 6 | 2.75 | 18 | 50 |
| | PRIONEX_3 DIL | | | | | | | | | | | | |
| 13 | CASEIN 1X | 0 | 6.25 | 10 | 0.95 | 3.38 | 5 | 0 | 6 | 0 | 2.75 | 15.67 | 50 |
| 14 | DMMA_0.018% + | 0 | 6.25 | 10 | 0.95 | 3.38 | 0 | 5 | 6 | 0 | 2.75 | 15.67 | 50 |
| | CASEIN_1X | | | | | | | | | | | | |
| 15 | PRIONEX_4 DIL | 0 | 6.25 | 10 | 0.95 | 3.38 | 5 | 0 | 0 | 6 | 2.75 | 15.67 | 50 |
| 16 | DMMA_0.018% + | 0 | 6.25 | 10 | 0.95 | 3.38 | 0 | 5 | 0 | 6 | 2.75 | 15.67 | 50 |
| | PRIONEX_4 DIL | | | | | | | | | | | | |

Here, different concentrations of DMMA, i.e., 0.018% and 0.008% were tested in combination with different dilutions of casein and Prionex in dry tubes and compared with DM version 1. Appropriate controls were used in the experiment. Effect of DMMA was tested using two Supernova™ conjugates, CD20-SN-v605 and HLADR-SN-v786. Specimen were processed using Protocol of Example 1B. Experiment was carried out on 4 donors.

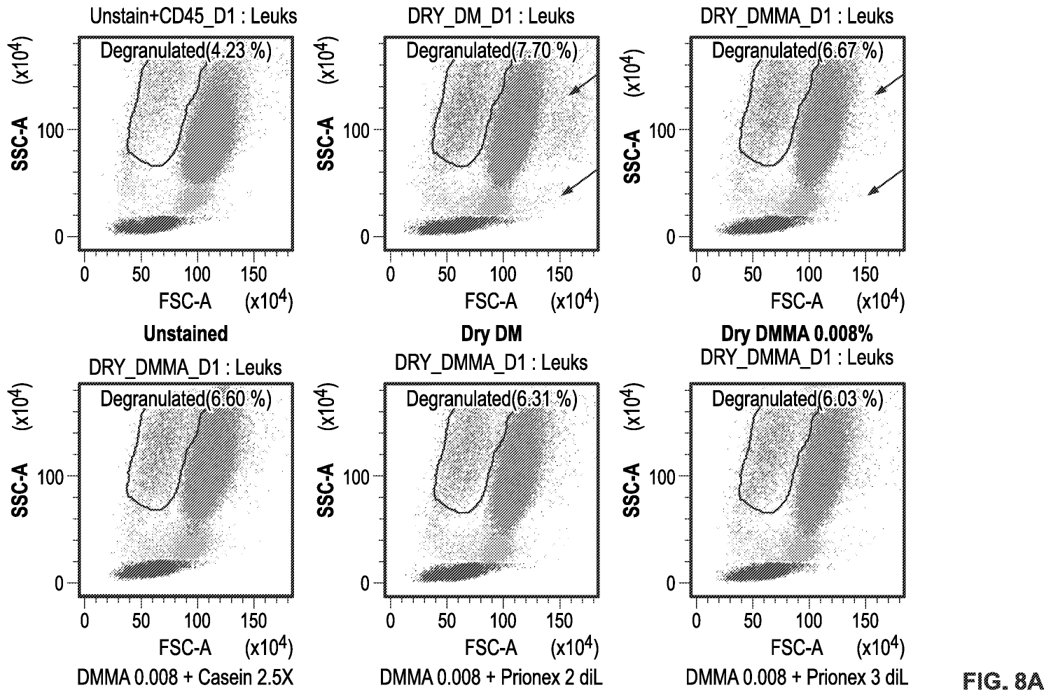

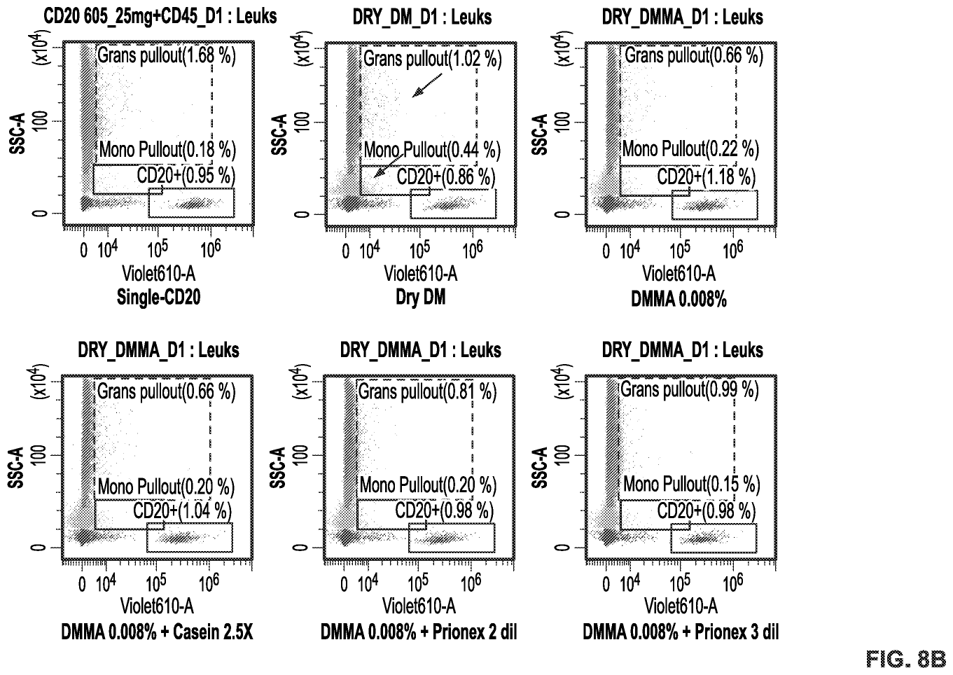

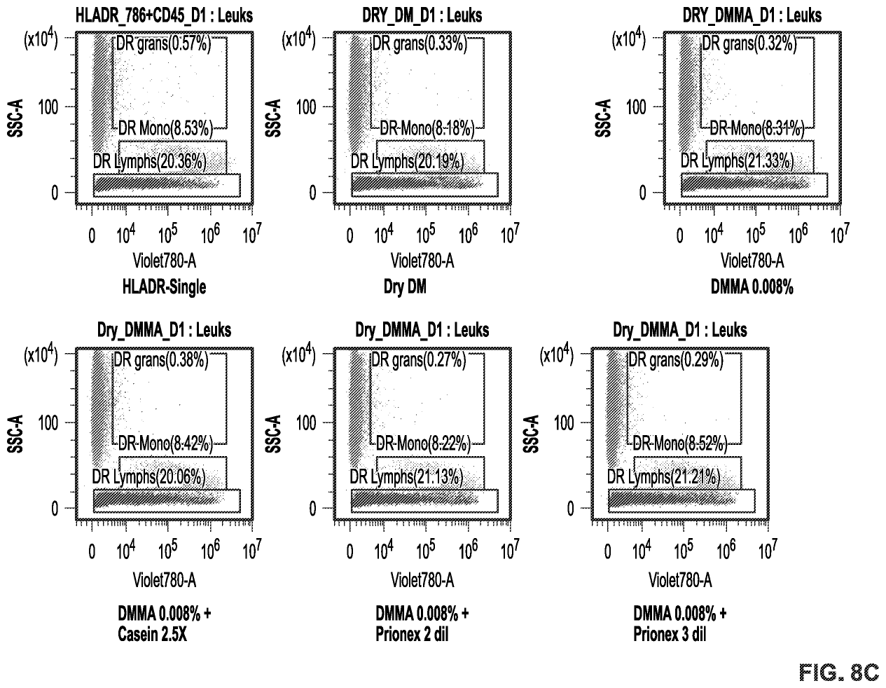

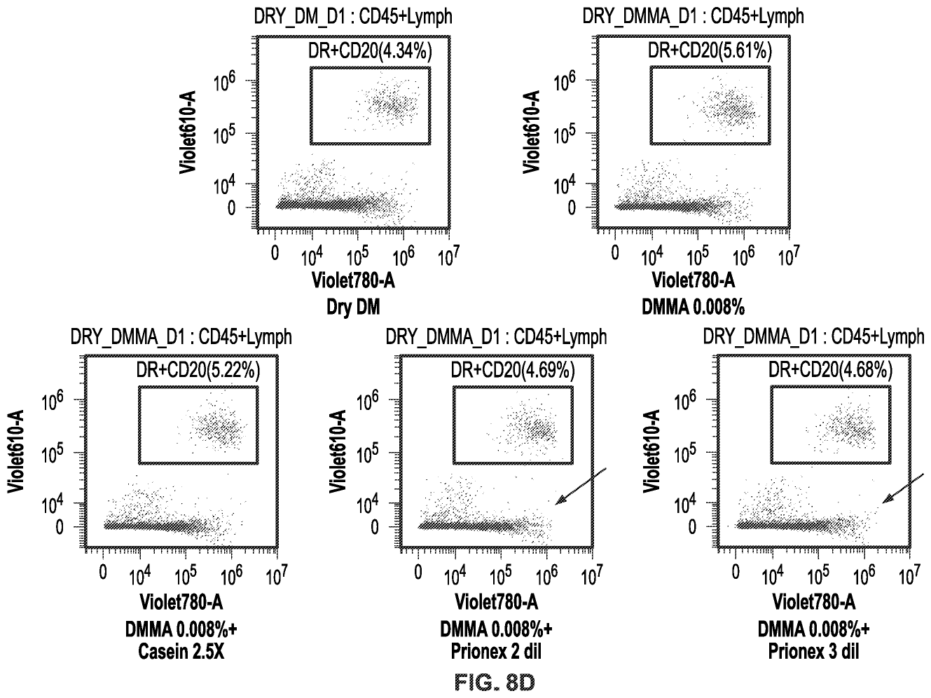

Results and observations: Flow plots of scatter properties, CD20-SN-v605, HLADR-SN-v786 and dual positive population for combination of additives with DMMA 0.008% were compared. FIG. 8A shows scatter plots of combinations of additives with DMMA 0.008% and compared with DM. Scatter properties look similar for each of the combinations, except DM. DM shows non-specific granulocyte and monocyte pullout as indicated with arrows. Similarly, DM shows higher non-specific granulocyte and monocyte pullout in V610 channel (indicated with arrow, FIG. 8B) as compared to other combination with 0.008% DMMA. % recruitment of CD20+ and HLADR+ found to be similar in all the combination with 0.008% DMMA compared to respective liquid singles. (FIG. 8B, FIG. 8C). Similarly, % recruitment of dual positive population in all the combination with 0.008% DMMA was found to be similar as compared to DM.

Similarly, flow plots of scatter properties, CD20-SN-v605, HLADR-SN-v786 and dual positive population were compared for combination of additives with DMMA 0.018%. (data not shown). Scatter plots of all the combination of additives with DMMA 0.018% were compared with DM. Scatter properties looks similar for all the combinations. DM and DMMA 0.018+casein 1× shows higher non-specific monocyte pullout in V610 channel, as compared to other combinations with 0.018% DMMA. % recruitment of CD20+ and HLADR+ were found to be similar in each of the combinations with 0.018% DMMA compared to respective liquid singles. Similarly, % recruitment of dual positive population in all the combination with 0.018% DMMA was found to be similar as compared to DM. However, DMMA 0.018%+casein 2.5× and 0.018% DMMA with Prionex combination showed less spread of 786+ events in V610 as compared to DM.

Flow plots of scatter properties, CD20-605, HLADR-786 and dual positive population for different concentration of single additives such as casein and Prionex (gelatin replaced by casein and Prionex in DM version 1) were compared (data not shown). Scatter properties appear similar for each of the combinations. DM alone with all the singles shows higher non-specific monocyte pullout in V610 channel as compared to a combination of additives with DMMA. % recruitment of CD20+ and HLADR+ found to be similar in all the singles compared to respective liquid singles. Similarly, % recruitment of dual positive population in each of the singles was found to be similar as compared to DM. Singles with Prionex dilutions shows less spread of 786+ events in V610 channel as compared to DM.

Overall, among all the combinations, DMMA 0.008%+prionex 2 dil and 3 dil, DMMA 0.018%+Prionex 3 dil and DMMA 0.018%+casein 2.5× provided good performance in terms of reducing non-specific monocyte pullout in V610 channel as well as reducing non-specific interaction between SN conjugates. This example demonstrates that combinations including DMMA 0.008%+prionex 2 dil and 3 dil, DMMA 0.018%+Prionex 3 dil and DMMA 0.018%+casein 2.5× provided good performance in terms of reducing non-specific binding and non-specific interaction when tested with two SN conjugates.

From these experiments, DM buffer with DMMA 0.008%+Prionex 2 dilution, and DMMA 0.018%+casein 2.5× dilution showed good performance in prevention of non-specific interaction between SN conjugates and also the non-specific pull-out. However, during development it was found out that DMMA 0.008%+Prionex 2 dilution (named as DM2 formulation) exhibited stability issues and hence was not evaluated further. Subsequent experiment with DMMA 0.018%+casein 2.5× (named as DM2+S) showed that this formulation has good stability: 6 months real time stability of dry tubes established so far (Study ongoing).

### 6: Functional Testing of Additives, Additive Minus One (AMO) in the Final Formulation (DM2+S)

AIM: To understand the effect of each additive present in DM2+Stabilizer buffer (final formulation) on the scatter properties and non-specific interactions, buffer minus one of each of the components was analyzed.
Method: Experiment was performed using four color panel (CD45-AA750, CD56-SNv428, CD20-SNv605 CD4-SNv786) in the below mentioned dried formulations. DM2+S served as a control in this experiment to compare with other groups.
1. DM2+S
2. DM2+S No monomer
3. DM2+S No DMMA
4. DM2+S No Casein
5. DM2+S Trehalose+Monomer
6. DM2+S Trehalose+DMMA
7. DM2+S Trehaloset Casein

It was established from previous experiments that trehalose is necessary for dry down, hence DM2+S without trehalose was not used for this experiment. Experiment was performed on 6 donors with single repeat. Sample processing protocol was followed as mentioned in the protocols and methods. Stop gate was set on 10,000 CD45+ lymphs. After dry down, dry tubes from all the groups were observed physically for any obvious changes. In addition, scatter properties, dual fluorescence plots were also observed for any non-specific pullout and non-specific interaction.

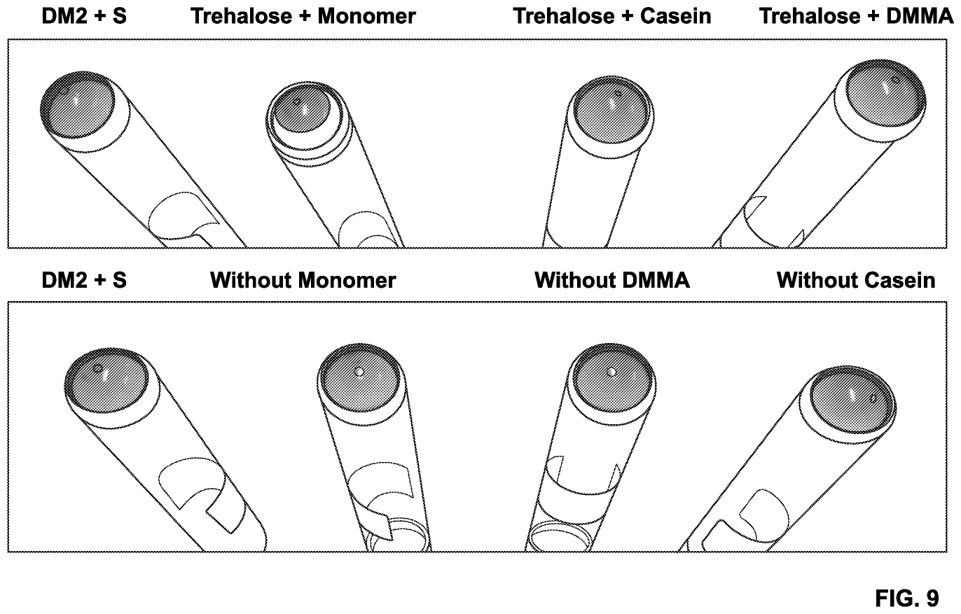

FIG. 9 shows physical appearance and properties of dry tubes in each of the tested groups DM2+S, Trehalose+Monomer, Trehalose+Casein, Trehalose+DMMA (left to right, upper panel), DM2+S, DM2+S without Monomer, DM2+S without DMMA, DM2+S without casein (left to right, lower panel). Here, DM2+S serves as control group. Physical observation indicates that without monomer, there is change in color of dry film (typically red film becomes light orange to brown). Shrinkage of film was observed in groups without casein and DMMA. No change in appearance of dry film was observed in tubes without DMMA compared to DM2+S. However, tubes without casein shows marginal shrinkage of dry film.

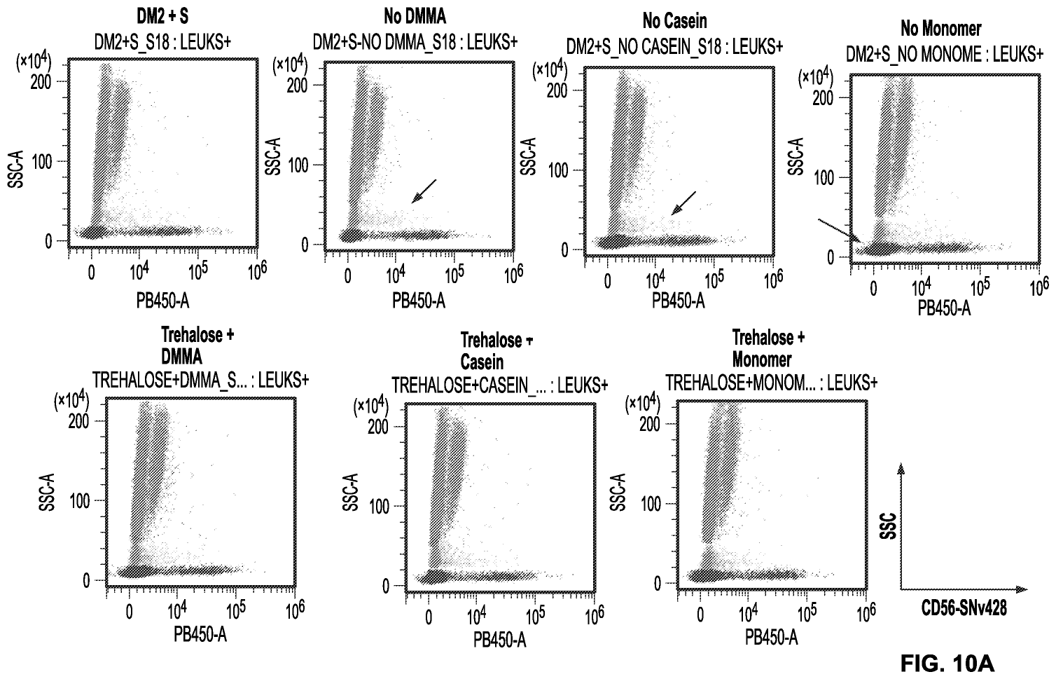

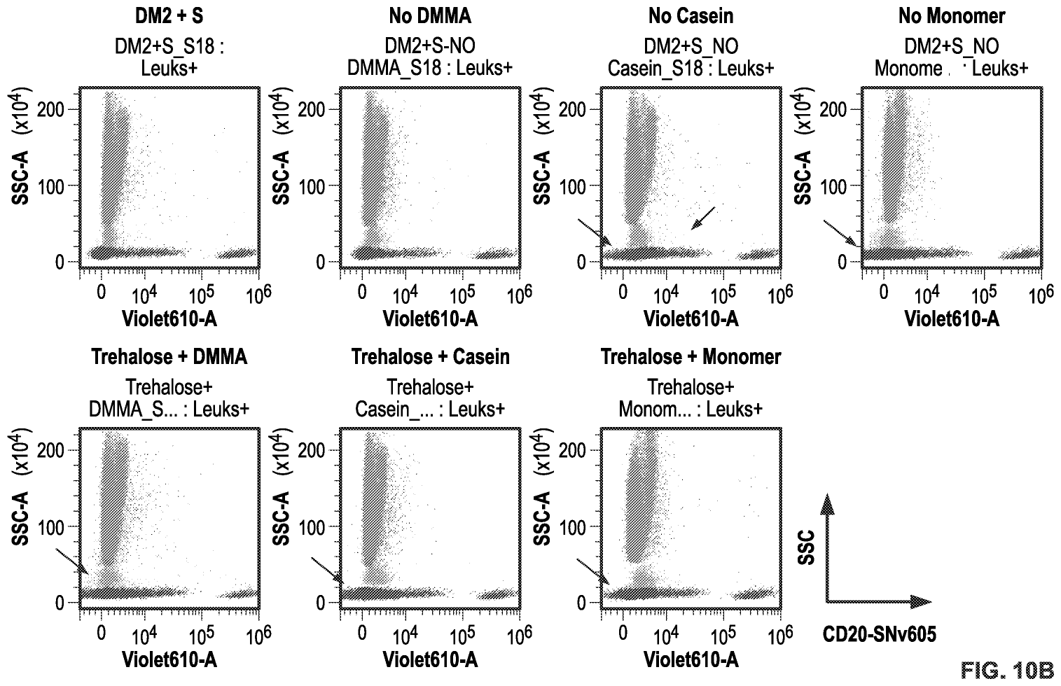

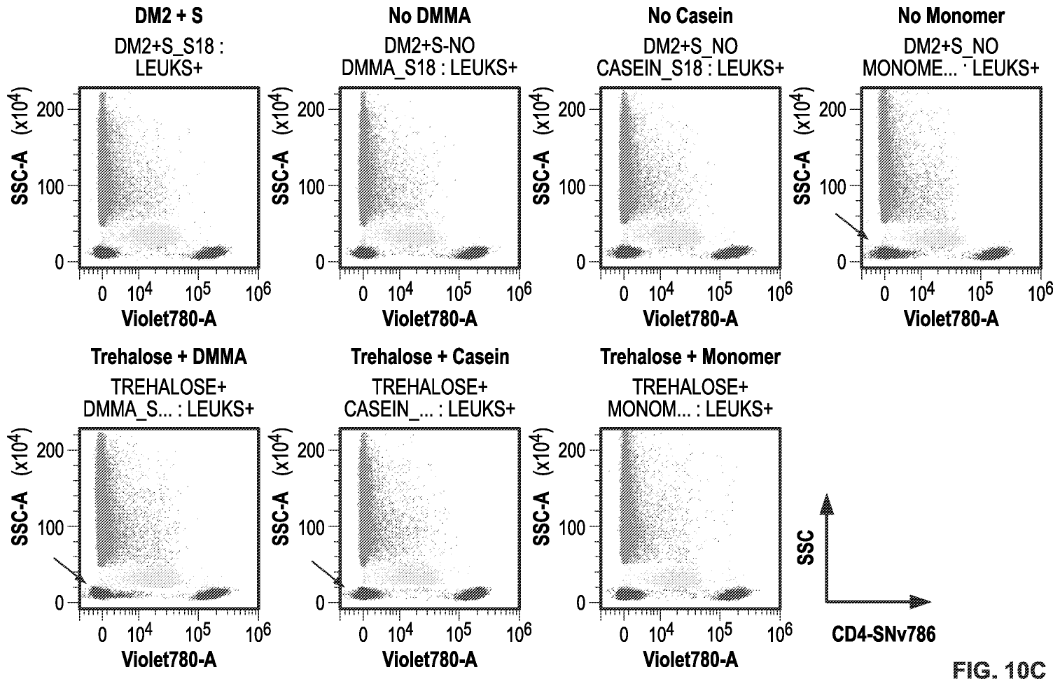

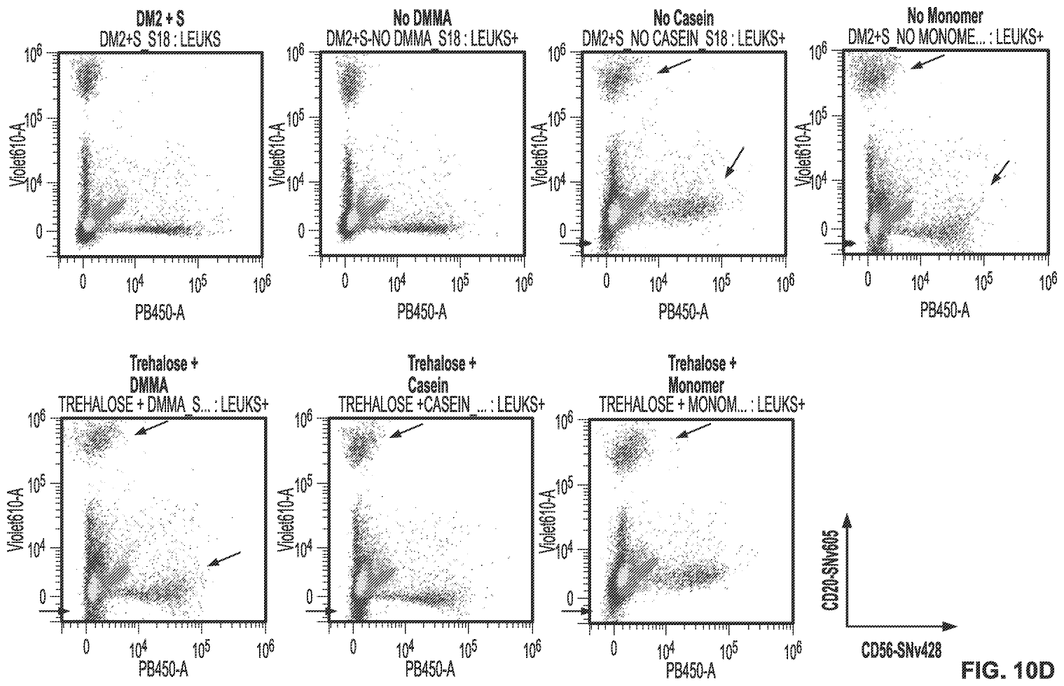

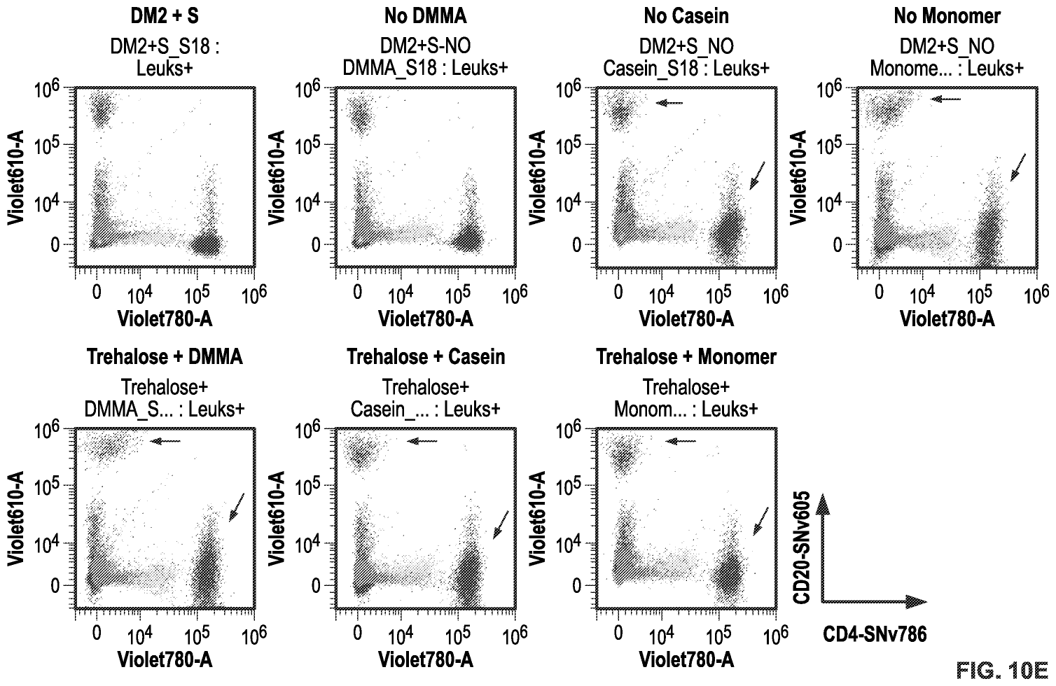

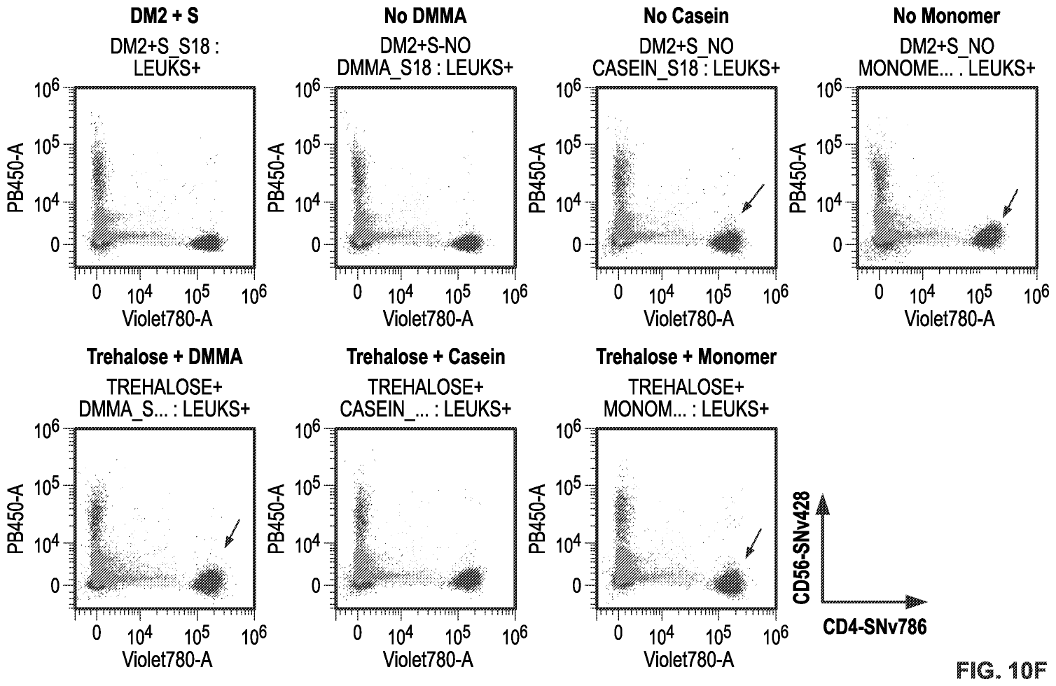

FIG. 10A-C show side scatter SSC vs FL plots for CD56-SNv428; CD20-SNv605, and CD4-SNv786, respectively, in tested groups DM2+S, DM2+S without DMMA, DM2+S without casein, DM2+S without Monomer (left to right, top panel), DM2+S: Trehalose+DMMA, DM2+S: Trehalose+Casein, DM2+S: Trehalose+Monomer (left to right, bottom panel). It can be observed from the figure that when casein and DMMA are not present, there is non-specific monocyte pullout (indicated with arrows). Absence of monomer causes spread of negative population in lymphocyte (indicated with arrows). Spread of negative population can be mainly attributed to non-specific interaction among SN dyes in absence of monomer and casein.

FIG. 10D-F show FL vs FL dual fluorescence plots (e.g., CD56-SNv428 vs CD20-SNv605, CD4-SNv786 vs CD20-SNv605, and CD4-SNv786 vs CD56-SNv428, respectively) for all the SN combinations in all the groups tested demonstrated that absence of monomer and casein cause non-specific interaction/spread of population between SN conjugates among the SN population. Hence, both monomer and casein are deemed important to prevent non-specific interaction.

This example demonstrates that monomer A and casein are important for dry down of SN conjugates, as without them inefficient prevention of cell-cell interaction is observed. In addition, casein also prevent non-specific monocyte pull-out. DMMA has main role in prevention of non-specific monocyte pull-out. This experiment not only provides function of individual additives, it is also important for troubleshooting in quality control issues.

### 7: Performance of DM2+S Dry Tubes in Comparison to BD Stain Buffer

AIM: To demonstrate performance of DM2+S dry tubes in comparison to BD stain buffer
Method: This experiment was performed to check performance of dry tubes when compared to performance of BD stain buffer. Here, performance of DM2+S dry tube was compared with BD horizon brilliant buffer. Experiment was performed using 4 color panel. Details of groups are as follow.

Three polymer dye conjugates along with gating marker (CD45-APC-A750, CD56-SNv428, CD20-SNv605, and CD4-SNv786) were evaluated using commercially available BD Horizon™ Brilliant stain buffer (Becton, Dickinson and Company). Staining protocol was followed as per the manufacturer's instruction. First, 50 μl of BD horizon brilliant buffer was added to tube, followed by addition of the 4 conjugates. Mix thoroughly by vortexing. Subsequently, 100 μl of blood specimen was added. Mix properly by vortexing, and incubate for 30 mins at room temperature and processed as mentioned in the protocols and methods section by following step 2 onwards.

Three polymer dye conjugates along with gating marker (CD45-APC-A750, CD56-SNv428, CD20-SNv605, and CD4-SNv786) were dried in inventive DM version 2+Stabilizer. Sample processing protocol is same as above mentioned in protocols and methods.

6 donors (with single repeat) were tested with the above protocol on all two mentioned groups Stop gate was set on 10,000 CD45+ lymphs.

### Results and Observations:

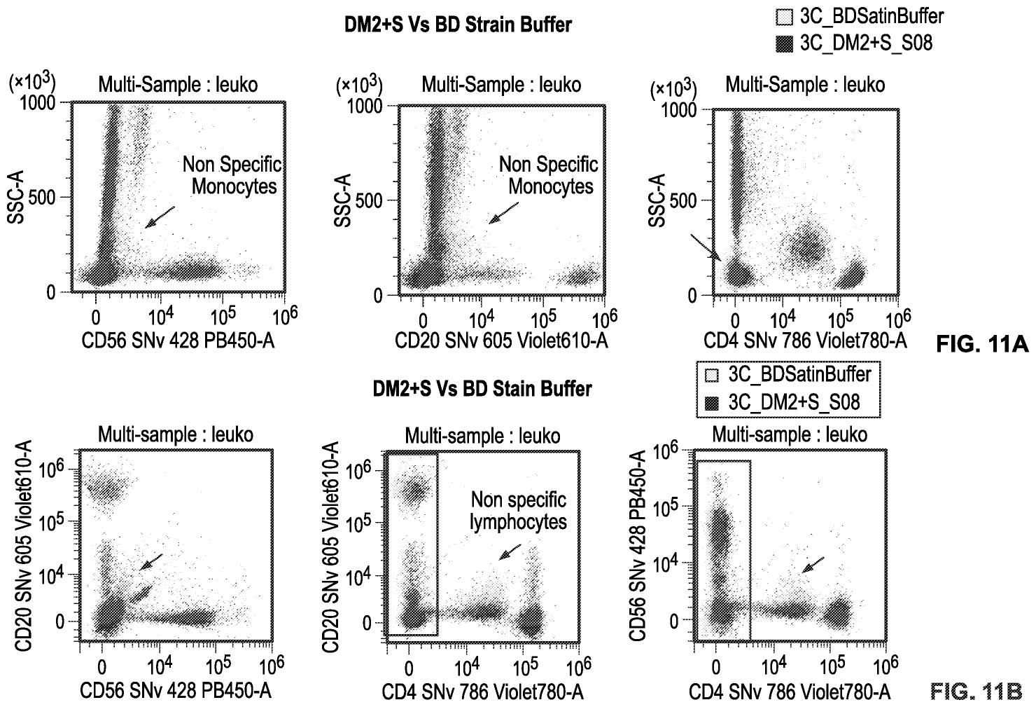

FIG. 11A shows representative SSC vs FL overlay flow plots for three polymer dye conjugates CD56-SNv428, CD20-SNv605, and CD4-SNv786 dried with either inventive DM2+S dry down buffer and reconstituted with blood sample, or with comparative BD Horizon™ Brilliant Stain buffer. Comparative commercial BD Horizon™ Brilliant stain buffer caused non-specific granulocyte and monocyte pullout (indicated with arrow) as compared to DM2+S dry tubes.

FIG. 11B shows representative dual fluorescence overlay plots for three polymer dye conjugates along with gating marker (CD45-APC-A750, CD56-SNv428, CD20-SNv605, and CD4-SNv786). The comparative BD Horizon™ Brilliant stain buffer caused non-specific lymphocyte pull-out in all the combination of SN conjugates, when compared to DM2+S.

This example demonstrates that DM2+S and BD stain buffers show no significant difference for the scatter properties, % recruitment and MdFI values (data not shown). However, DM2+S dry tubes show tighter population (without any non-specific pull out) when compared to BD stain buffer. Hence, it can be concluded from the data that DM2+S dry tubes has better performance as compared to BD stain buffer.

### Example 4. 6-Month Interim Stability

Aim: To assess and check 6-month stability of DM2+S dry tubes
Method: In this example, 6-month stability of DM2+S dry tubes was evaluated. For comparison, 3-month old as well as fresh lots were included in the study. Details of each lot is given in Table 9. Both 3-month and 6-month old dry tubes tested in the study belong to open pouch, i.e. dry tubes were tested from the pouch opened multiple times. These dry tubes contain 12 color (12C) panel (9C conventional conjugates+3C SN conjugates, CD56-SNv428, CD20-SNv605, CD4-SNv786). These lots were tested on 4 donors with single repeat on single instrument. Stain-lyse-wash protocol mentioned in Example 1B was used for the study. Stop gate was set on 10,000 CD45+ lymphocytes. Compensation was adjusted for fresh lot. Subsequently, compensation of fresh lot was applied to 3-month and 6-month old lots and required minor compensation adjustments were done in both 3-month and 6-month old lots. Absolute % delta recruitment and MdFI of 3-month and 6-month old lots were compared with fresh lot (data not shown).

TABLE 9
Details of lots used in stability studies

| Lot | Lot size (tests) | Date of lot building |
|---|---|---|
| Fresh lot | 25 | 27-05-2021 |
| 3-Month old | 870 | 08-03-2021 |
| 6-Month old | 50 | 08-12-2020 |

### Results and Observations:

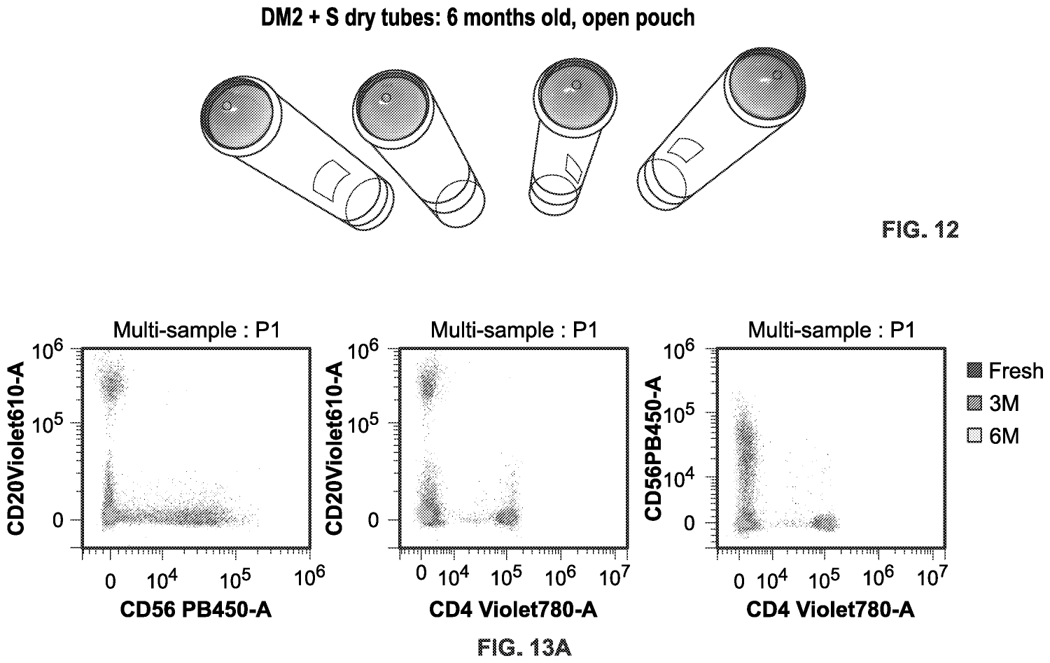

FIG. 12 shows photographic images of tubes with dry down film in 6 months old dry tubes. No apparent deterioration was visually observed in the dry down film.

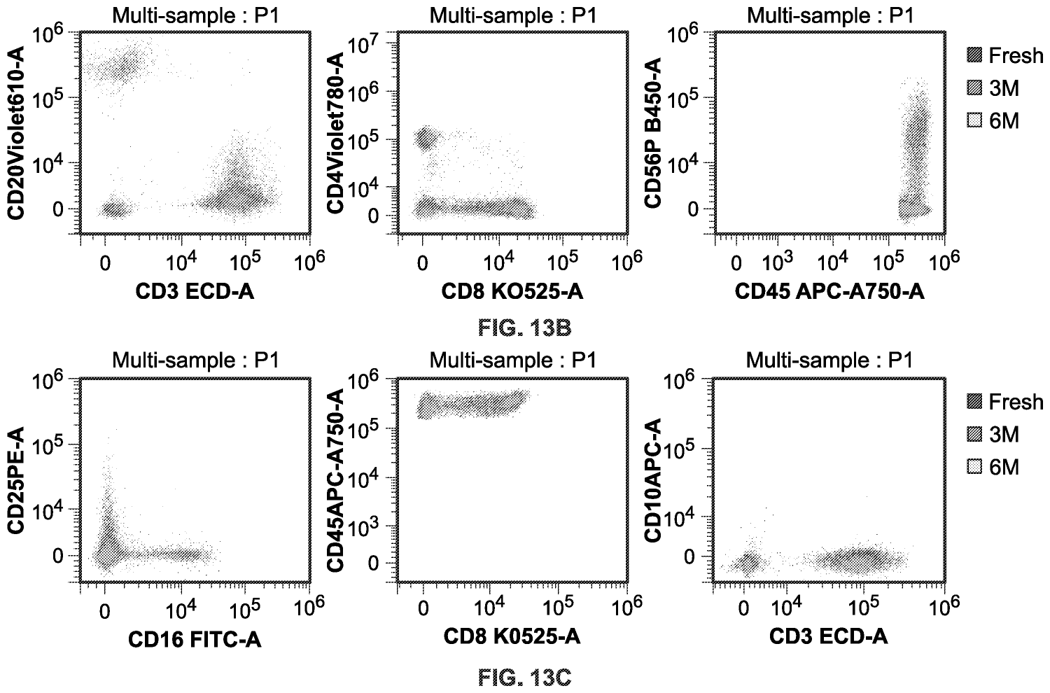

FIGS. 13A, 13B, and 13C show representative overlay plots of all 3 lots for different combinations of specificities tested in the study.

FIG. 13A shows DM2+S dry tubes representative FL vs FL overlay plots for 6-months stability, compared to 3-months and fresh lots. Dry tubes containing a 12 color (12C) panel (9C conventional conjugates+3C SN conjugates, CD56-SNv428, CD20-SNv605, CD4-SNv786) were tested on 4 donors with single repeat on single flow cytometry instrument. Representative FL vs FL overlay plots for different combination of SN dyes in all the three lots for CD56 PB450-A vs CD20 Violet610 (left panel), CD4Violet780 vs CD20Violet610 (middle panel), and CD4 Violet780 vs CD56PB450 (right panel). These overlays show that population in 3-month and 6-month old lots completely overlay with fresh lot. In addition, no non-specific interaction or spread of population was observed in 6-month old lot in all the tested donors.

FIG. 13B shows DM2+S dry tubes 6-months stability compared to 3-months, and fresh lots: Representative FL vs FL overlay plots for SN vs Classical combination in all the three lots. Dry tubes containing a 12 color (12C) panel (9C conventional conjugates+3C SN conjugates, CD56-SNv428, CD20-SNv605, CD4-SNv786) were tested on 4 donors with single repeat on single flow cytometry instrument. Overlays for 6-months, 3-months, and fresh lots for CD3 ECD vs CD20Violet610 (left panel), CD8 KO525 vs CD4Violet780 (middle panel), and CD45 APC-A750 vs CD56PB450 (right panel) show that population in 3-month and 6-month old lots completely overlay with fresh lot. In addition, no non-specific interaction or spread of population was observed in 6-month old lot in all the tested donors.

FIG. 13C shows DM2+S dry tubes 6-months stability compared to 3-months and fresh lots: Representative FL vs FL overlay plots for Classical vs Classical combination in all the three lots. Dry tubes containing a 12 color (12C) panel (9C conventional conjugates+3C SN conjugates, CD56-SNv428, CD20-SNv605, CD4-SNv786) were tested on 4 donors with single repeat on single flow cytometry instrument. Overlays for 6-months, 3-months, and fresh lots for CD16 FITC vs CD25PE (left panel), CD8 KO525 vs CD45APC-A750 (middle panel), and CD3 ECD vs CD10APC (right panel) show that population in 3-month and 6-month old lots completely overlay with fresh lot. In addition, no non-specific interaction or spread of population was observed in 6-month old lot in all the tested donors. In this example, 6 month interim stability results indicate that DM2+S dry tubes are stable for at least 6 months. No non-specific interaction or spread was observed in interim 6-months time point. Real time stability is planned for 24 months.

### Example 5. Standardized DM Buffer Formulation

The inventive DM Buffer may be prepared as follows. Stock preparation of solubilized additives (components) may be prepared as follows. After stock preparation, DM2+S buffer is prepared as shown in the Table 10. The pH range of DM2+S buffer was found to be between 7 to 7.4.

TABLE 10
DM Buffer (DM2 + S) Formulation
Preparation of DM buffer

| | | | | | Enter the Number | |
|---|---|---|---|---|---|---|
| | | | | | of tests = 30 | |
| | | | | | Volume | Pick Up |
| | | | | | per test | Volume |
| Component | Stock Conc | Solvent | Vendor | PN | (uL) | (in uL) |
| Trehalose | 400 mg/ml | 1x PBS | Sigma | T9531 | 6.25 | 187.5 |
| Monomer A | 40 mg/ml | DI water | Lumigen | MOA-0010 | 10 | 300 |
| DMMA | 1.5 mg/ml | DI water | Sigma | T7763 | 6 | 180 |
| Casein | 2.5x dilution from | 1x PBS | Sigma | B6429 | 6 | 180 |
| | 10x stock | | | | | |
| Na Azide | 10% | DI water | Sigma | 10199-00 | 0.05 | 1.5 |
| Allura Red | 50 mg/ml | DI water | Sigma | 458848 | 0.18 | 5.4 |
| Total | | | | | 28.48 | 854.4 |

Subsequently, DM2+S DM buffer (Table 10) was used to prepare a panel formulation as shown in the Table 11 as a single dried reactant uniform film in a tube format comprising three fluorescent polymer dye conjugates.

TABLE 11
Preparation of Fluorescent Dye-Antibody Panel
Formulation (12 color) using DM2 + S Buffer

| | | | | Enter No. | |
|---|---|---|---|---|---|
| | | | | of Tests: | 25 |
| | Dose | | Concentration | Volume per | Vol |
| Conjugate | (μg/test) | PN | (μg/uL) | test (μL) | (Pick Up) |
| CD16-FITC | 0.25 | 2JF-3GB-FIT | 1.987 | 1.987 | 3.15 |
| CD25-PE | 0.25 | 2JF-B1_49-RPE | 0.268 | 0.933 | 23.32 |
| CD3-ECD | 0.2 | 2JF-UCHT1-ECD | 2.056 | 0.097 | 2.43 |
| CD38-PC5.5 | 0.0625 | 2JF-LS 198-P55 | 0.282 | 0.222 | 5.54 |
| CD27-PC7 | 0.3 | 2JF-1A4LDG- | 0.301 | 0.997 | 24.92 |
| | | PC7 | | | |
| CD10-APC | 0.125 | 2JF-ALB1-APC | 0.489 | 0.256 | 6.39 |
| CD14-APCA700 | 0.25 | 2JF-RMO52-A70 | 0.23 | 1.087 | 27.17 |
| CD45-AA750 | 1 | 2JF-J33-A75 | 0.452 | 2.212 | 55.31 |
| CD8-KRO | 0.125 | 2JF-B9_11-KRO | 0.75 | 0.167 | 4.17 |
| CD56-SNv428 | 0.125 | C76803 | 0.374 | 0.334 | 8.36 |
| CD20-SNv605 | 1 | C78079 | 0.239 | 4.184 | 104.60 |
| CD4-SNv786 | 0.25 | C281020 | 0.304 | 0.822 | 20.56 |
| DM ver 2 | | | | 28.48 | 712.00 |
| 1 × PBS | q.s | | | 9.44 | 252.08 |
| | | | | Total | 1250 |

The final volume per test is 50 uL.

1. A buffer composition for use in drying a plurality of dye conjugates on a substrate, comprising
a water-soluble monomer comprising an aryl moiety or heteroaryl moiety;
a protein stabilizer;
a carbohydrate stabilizer; and
a zwitterionic surfactant,
optionally wherein the composition is an aqueous composition, further optionally wherein the composition has a pH within a range of pH 6.5-7.5, or pH 7.0-7.4.

2. The buffer composition according to claim 1, wherein the plurality of dye conjugates comprises at least one polymer dye conjugate, optionally wherein the plurality of dye conjugates comprises two or more, three or more, four or more, five or more, six or more, seven or more, eight or more, nine or more, ten or more, 11 or more, 12 or more, or 2 to 20, 3 to 18, or 4 to 12 different dye conjugates each comprising a different binding partner, further optionally wherein the composition is in the form of a single reactant film disposed on a substrate.

3. The buffer composition according to claim 2, wherein the polymer dye conjugate is a binding partner conjugated to a fluorescent polymer dye having a structure according to Formula (IV): 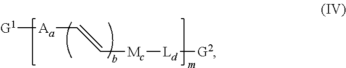
wherein:
each A is independently selected from the group consisting of an aromatic co-monomer and a heteroaromatic co-monomer;
each L is a linker moiety;
each M is independently selected from the group consisting of an aromatic co-monomer, a heteroaromatic co-monomer, a bandgap-modifying monomer, optionally substituted ethylene, and ethynylene;
G¹ and G² are independently selected from an unmodified polymer terminus and a modified polymer terminus;
a, c, and d independently define the mol % of each unit within the structure which each can be evenly or randomly repeated and where each a is a mol % from 10 to 100%, each c is a mol % from 0 to 90%, and each d is a mol % from 0 to 25%;
each b is independently 0 or 1; and
m is an integer from 1 to about 10,000, optionally wherein the plurality of dye conjugates comprises two or more different fluorescent polymer dye conjugates each having a structure according to formula (IV).

4. (canceled)

5. The buffer composition according to claim 3, wherein the water-soluble monomer is a co-monomeric unit A used in preparation of at least one of the fluorescent polymer dye conjugates, optionally wherein the water-soluble monomer is a dihydrophenanthrene (DHP)-based water-soluble monomer or a fluorene-based water-soluble monomer.

6. (canceled)

7. The buffer composition according to claim 5, wherein the dihydrophenanthrene (DHP)-based water-soluble monomer has a chemical structure according to Formula (I) and the fluorene-based water-soluble monomer having a chemical structure according to Formula (II): 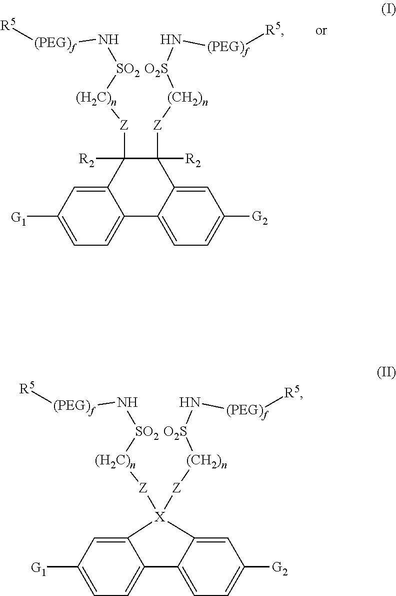
wherein
each X is C or Si;
each G₁, G₂ is independently selected from the group consisting of halogen, alkyl, PEG, hydrogen, alkyne, optionally substituted aryl, optionally substituted heteroaryl, halogen substituted aryl, silyl, diazonium salt, triflate, acetyloxy, azide, sulfonate, phosphate, optionally substituted tetrahydropyrene (THP), optionally substituted fluorene, optionally substituted dihydrophenanthrene (DHP), aryl or heteroaryl substituted with one or more pendant chains terminated with a functional group selected from amine, carbamate, carboxylic acid, carboxylate, maleimide, activated ester, N-hydroxysuccinimidyl, hydrazine, hydrazide, hydrazone, azide, alkyne, aldehyde, and thiol;
each R² is independently selected from the group consisting of a water-solubilizing moiety, alkene, alkyne, cycloalkyl, haloalkyl, (hetero)aryloxy, (hetero)arylamino, sulfonamide-PEG, phosphoramide-PEG, ammonium alkyl salt, ammonium alkyloxy salt, ammonium oligoether salt, sulfonate alkyl salt, sulfonate alkoxy salt, sulfonate oligoether salt, sulfonamido oligoether, sulfonamide, sulfinamide, phosphonamidate, phosphinamide, 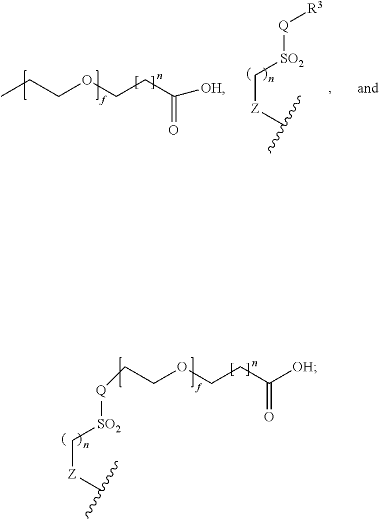
each R³ is a water-solubilizing moiety;
each R⁴ is independently selected from the group consisting of H, alkyl, PEG, a water-solubilizing moiety, a linker moiety, a chromophore, carboxylic amine, amine, carbamate, carboxylic acid, carboxylate ester, maleimide, activated ester, N-hydroxysuccinimidyl, hydrazine, hydrazide, hydrazone, azide, alkyne, aldehyde, or thiol, or protected groups thereof;
each R⁵ is independently selected from the group consisting of H, hydroxyl, C₁-C₁₂ alkyl, C₂-C₁₂ alkene, C₂-C₁₂ alkyne, C₃-C₁₂ cycloalkyl, C₁-C₁₂ haloalkyl, C₁-C₁₂ alkoxy, C₂-C₁₈ (hetero)aryloxy, C₂-C₁₈ (hetero)arylamino, C₂-C₁₂ carboxylic acid, and C₂-C₁₂ carboxylate ester;
each Q is independently a bond, NR⁴, or —CH₂;
each Z is independently CH₂, O, or NR⁴;
each f is independently an integer from 0 to 50; and
each n is independently an integer from 1 to 20,
optionally wherein each G₁, G₂ is halogen; each Z is O; each R² is H; each R$ is H, hydroxyl, C₁-C₆ alkyl, or C₁-C₆ alkoxy; each n is independently 2-4; and each f is independently 5-20,
further optionally wherein each n is 3; each R⁵ is —OCH₃; and each f is 11-12.

8.-11. (canceled)

12. The buffer composition according to claim 1, wherein the zwitterionic surfactant has a structure according to Formula (XV): 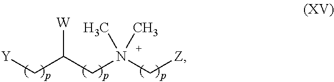
wherein Y═CO₂—, or SO₃—, W═H or OH, Z═CH₃ or NHC(O)R, wherein R═C₁₋₁₅ alkyl; independently each p=0 or 1; and q=0-21; optionally wherein W═H, Z═CH₃, and q=11-15,
optionally wherein the zwitterionic surfactant is selected from the group consisting of 3-(N,N-dimethylmyristylammonio propane sulfonate (DMMA); 3-[N,N-dimethyl(3-palmitoylaminopropyl) ammonio]-propane sulfonate (DMPA); N-(alkyl C₁₀-C₁₆)—N,N-dimethylglycine betaine; and N,N-dimethyl-N-dodecylglycine betaine.

13. (canceled)

14. The buffer composition according to claim 1, wherein the protein stabilizer is selected from the group consisting of a casein, a bovine serum albumin (BSA), and a gelatin.

15. The buffer composition according to claim 1, wherein the carbohydrate stabilizer is a disaccharide carbohydrate stabilizer, optionally wherein the disaccharide carbohydrate stabilizer is a trehalose or a hydrate thereof, optionally wherein the trehalose or hydrate thereof is trehalose dihydrate.

16. (canceled)

17. The buffer composition according to claim 1 further comprising one or more additional additives selected from the group consisting of a preservative, antioxidant, anionic surfactant, nonionic surfactant, and a colorant.

18.-19. (canceled)

20. The buffer composition according to claim 1, comprising per test:
200 to 800 μg of the water-soluble monomer;
2000 to 3000 μg of the carbohydrate stabilizer;
8.4 to 72 μg of the protein stabilizer; and
2 to 9 μg of the zwitterionic surfactant.

21. A method of preparing a single reactant film, the method comprising:
dispensing onto a substrate a plurality of dye conjugates together in a liquid phase comprising an aqueous buffer composition according to claim 1, the plurality of dye conjugates comprising at least one polymer dye conjugate, optionally wherein the plurality of dye conjugates comprises two or more, three or more, four or more, five or more, six or more, seven or more, eight or more, nine or more, ten or more, 11 or more, 12 or more, or 2 to 20, 3 to 18, or 4 to 12 different dye conjugates each comprising a different binding partner; and
drying the plurality of dye conjugates together in the liquid phase aqueous buffer to form a first single reactant film on the substrate, optionally wherein the substrate is selected from the group consisting of a tube, well, membrane, bead, and an inside surface of a reaction vessel.

22.-25. (canceled)

26. The method according to claim 21, wherein exposing the first single reactant film to a first aliquot of a liquid blood sample, processing, and analyzing by flow cytometry to obtain a first flow cytometry plot exhibiting one or more of the group consisting of:
decreased non-specific binding of monocytes;
decreased non-specific binding of granulocytes;
decreased non-specific interaction of polymer dye conjugates; and
decreased aggregation of polymer dye conjugates,
when compared to a second flow cytometry plot obtained by exposing a second single reactant film to a second aliquot of the liquid biological sample, processing, and analyzing by flow cytometry,
wherein the second single reactant film is prepared from a liquid phase comprising the same plurality of dye conjugates dried together to form the first single reactant film and prior drying technology Reagent Buffer without the water-soluble monomer and without the zwitterionic surfactant.

27. A composition comprising
one or more or a plurality of fluorescent polymer dye conjugates, optionally two or more, three or more, four or more, five or more, six or more, seven or more, eight or more, nine or more, ten or more, 11 or more, 12 or more, or 2 to 20, 3 to 18, or 4 to 12 different dye conjugates each comprising a different binding partner;
a water-soluble monomer comprising an aryl moiety or heteroaryl moiety, optionally comprising one or more water-solubilizing moieties attached thereto;
a protein stabilizer;
a carbohydrate stabilizer; and
a zwitterionic surfactant,
optionally wherein the composition is an aqueous composition, and further optionally wherein the composition has a pH within a range of pH 6.5-7.5, or pH 7.0-7.4.

28. The composition according to claim 27 in the form of a single reactant film disposed on a substrate, optionally wherein the substrate is selected from the group consisting of a tube, well, membrane, bead, and an inside surface of a reaction vessel.

29. The composition according to claim 27, comprising per single reactant film:
200 to 800 μg of the water-soluble monomer;
2000 to 3000 μg of the carbohydrate stabilizer;
8.4 to 72 μg of the protein stabilizer; and
2 to 9 μg of the zwitterionic surfactant.

30. The composition according to claim 27, wherein at least one of the fluorescent polymer dye conjugates comprises a fluorescent polymer dye moiety having a structure according to Formula (IV): 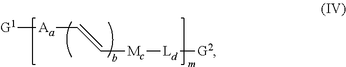
wherein:
each A is independently selected from the group consisting of an aromatic co-monomer and a heteroaromatic co-monomer;
each L is a linker moiety;
each M is independently selected from the group consisting of an aromatic co-monomer, a heteroaromatic co-monomer, a bandgap-modifying monomer, optionally substituted ethylene, and ethynylene;
G¹ and G² are independently selected from an unmodified polymer terminus and a modified polymer terminus;
a, c, and d independently define the mol % of each unit within the structure which each can be evenly or randomly repeated and where each a is a mol % from 10 to 100%, each c is a mol % from 0 to 90%, and each d is a mol % from 0 to 25%;
each b is independently 0 or 1; and
m is an integer from 1 to about 10,000,
optionally wherein the co-monomer is a dihydrophenanthrene (DHP)-based water-soluble monomer or a fluorene-based water-soluble monomer.

31.-32. (canceled)

33. The composition according to claim 30, wherein the dihydrophenanthrene (DHP)-based water-soluble monomer has a chemical structure according to Formula (I) and the fluorene-based water-soluble monomer has a chemical structure according to Formula (II): 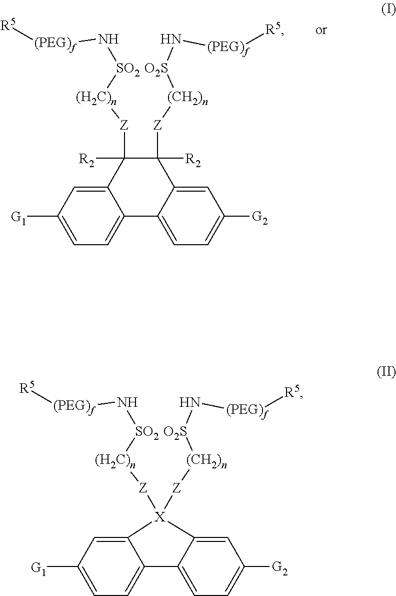
wherein
each X is C or Si;
each G₁, G₂ is independently selected from the group consisting of halogen, alkyl, PEG, hydrogen, alkyne, optionally substituted aryl, optionally substituted heteroaryl, halogen substituted aryl, silyl, diazonium salt, triflate, acetyloxy, azide, sulfonate, phosphate, optionally substituted tetrahydropyrene (THP), optionally substituted fluorene, optionally substituted dihydrophenanthrene (DHP), aryl or heteroaryl substituted with one or more pendant chains terminated with a functional group selected from amine, carbamate, carboxylic acid, carboxylate, maleimide, activated ester, N-hydroxysuccinimidyl, hydrazine, hydrazide, hydrazone, azide, alkyne, aldehyde, and thiol;
each R² is independently selected from the group consisting of a water-solubilizing moiety, alkene, alkyne, cycloalkyl, haloalkyl, (hetero)aryloxy, (hetero)arylamino, sulfonamide-PEG, phosphoramide-PEG, ammonium alkyl salt, ammonium alkyloxy salt, ammonium oligoether salt, sulfonate alkyl salt, sulfonate alkoxy salt, sulfonate oligoether salt, sulfonamido oligoether, sulfonamide, sulfinamide, phosphonamidate, phosphinamide, 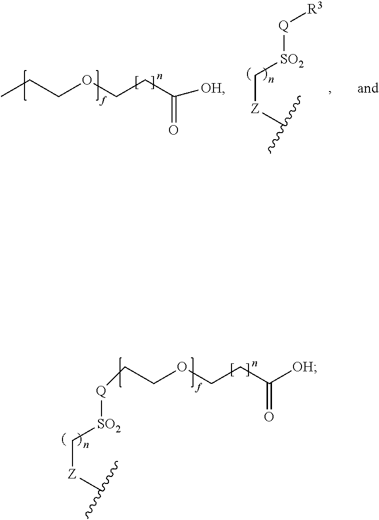
each R³ is a water-solubilizing moiety;
each R⁴ is independently selected from the group consisting of H, alkyl, PEG, a water-solubilizing moiety, a linker moiety, a chromophore, carboxylic amine, amine, carbamate, carboxylic acid, carboxylate ester, maleimide, activated ester, N-hydroxysuccinimidyl, hydrazine, hydrazide, hydrazone, azide, alkyne, aldehyde, or thiol, or protected groups thereof;
each R⁵ is independently selected from the group consisting of H, hydroxyl, C₁-C₁₂ alkyl, C₂-C₁₂ alkene, C₂-C₁₂ alkyne, C₃-C₁₂ cycloalkyl, C₁-C₁₂ haloalkyl, C₁-C₁₂ alkoxy, C₂-C₁₈ (hetero)aryloxy, C₂-C₁₈ (hetero)arylamino, C₂-C₁₂ carboxylic acid, and C₂-C₁₂ carboxylate ester;
each Q is independently a bond, NR⁴, or —CH₂;
each Z is independently CH₂, O, or NR⁴;
each f is independently an integer from 0 to 50; and
each n is independently an integer from 1 to 20.

34.-35. (canceled)

36. The composition according to claim 27, wherein the zwitterionic surfactant has a structure according to Formula (XV): 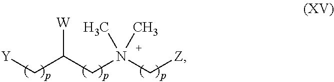
wherein Y═CO₂—, or SO₃—, W═H or OH, Z═CH₃ or NHC(O)R, wherein R═C₁₋₁₅ alkyl; independently each p=0 or 1; and q=0-21; optionally wherein W═H, Z═CH₃, and q=11-15; further optionally wherein the zwitterionic surfactant is selected from the group consisting of 3-(N,N-dimethylmyristylammonio propane sulfonate (DMMA); 3-[N,N-dimethyl (3-palmitoylaminopropyl) ammonio]-propane sulfonate (DMPA); N-(alkyl C₁₀-C₁₆)—N,N-dimethylglycine betaine; and N,N-dimethyl-N-dodecylglycine betaine.

37. (canceled)

38. The composition according to claim 27, wherein the protein stabilizer is selected from the group consisting of a casein, a bovine serum albumin (BSA), and a gelatin.

39. The composition according to claim 27, wherein the carbohydrate stabilizer is a disaccharide carbohydrate stabilizer, optionally wherein the disaccharide carbohydrate stabilizer is a trehalose or a hydrate thereof, optionally wherein the trehalose or hydrate thereof is trehalose dihydrate.

40.-41. (canceled)
